(19) United States
(12) Patent Application Publication
(10) Pub. No.: US 2024/0109879 A1
(43) Pub. Date: Apr. 4, 2024

(54) BENZIMIDAZOLE COMPOUND AND APPLICATION THEREOF

(71) Applicant: Medshine Discovery Inc., Nanjing, Jiangsu (CN)

(72) Inventors: Chunli Shen, Shanghai (CN); Yuchuan Zhu, Shanghai (CN); Jinxin Liu, Shanghai (CN); Chengde Wu, Shanghai (CN); Jian Li, Shanghai (CN); Shuhui Chen, Shanghai (CN)

(73) Assignee: Medshine Discovery Inc., Nanjing, Jiangsu (CN)

(21) Appl. No.: 18/269,901

(22) PCT Filed: Dec. 29, 2021
(86) PCT No.: PCT/CN2021/142486

§ 371 Date: Jun. 27, 2023

(30) Foreign Application Priority Data
Dec. 31, 2020 (CN) 202011643388.9

(51) Int. Cl. C07D 413/14; C07D 417/14

(52) U.S. Cl. CPC C07D 413/14; C07D 417/14

(57) ABSTRACT

Provided are a class of benzimidazole compounds and an application thereof as a 300/CBP inhibitor. Specifically, provided are a compound represented by formula (P) and a pharmaceutically acceptable salt thereof.

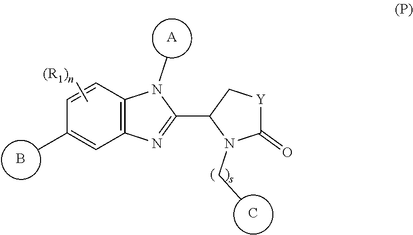

### CROSS REFERENCE TO RELATED APPLICATIONS

This application is a National Stage filing under 35 U.S.C. 371 of International PCT Application No. PCT/CN2021/142486, filed Dec. 29, 2021, which claims the priority of CN202011643388.9, filed on Dec. 31, 2020. The Chinese Patent Application No. CN202011643388.9 is incorporated herein by reference as part of the disclosure of the present application.

### FIELD OF THE INVENTION

The present disclosure relates to a class of benzimidazole compounds and use thereof, and specially relates to a compound represented by formula (P) or a pharmaceutically acceptable salt thereof.

### BACKGROUND OF THE INVENTION

A p300/CBP family, consisting of highly homologous HAT adenovirus E1A-related 300 kDa protein (adenoviral EIA binding protein of 300 kDa, p300) and cyclic adenosine monophosphate response element binding protein (CREB binding protein, CBP), is one of the major members of the HAT family. The p300/CBP is involved in cell cycle progression and cell growth, differentiation and development, and is a very important coactivator.

The p300 and CBP are positive regulators of cancer progression and are closely related to various human tumor diseases. Highly expressed p300 in breast cancer may promote a recurrence of a tumor and is related to aggressive features of breast cancer. High expression of p300 in hepatocellular carcinoma is associated with enhanced vascular invasion, intrahepatic metastasis and shortened threshold. In prostate cancer, androgen-induced androgen receptor (AR) recruitment to chromatin is closely related to H3K27 acetylation. By preventing the H3K27 acetylation, the function of p300/CBP as a coactivator on AR is prevented, thereby blocking the expression of key proliferation gene and the growth of tumor, and showing the potential of p300/CBP inhibitors in the field of the treatment of prostate cancer. Multiple studies have also shown that mutated p300/CBP is related to many hematological malignancies. The role of the epigenetic regulator p300/CBP in inducing and maintaining acute myeloid leukemia (AML) has been demonstrated by experiments in mouse models using gene knockout in vitro and in vivo or other experiments. Induction of cell cycle arrest and apoptosis using small molecule inhibitors of p300/CBP has efficacy in multiple AML subtypes. Acute lymphoblastic leukemia (ALL) is the most common childhood malignancy. A study has shown that p300/CBP is involved in recurrent ALL-related chromosomal translocation and is a key regulator of tumor cell growth.

### SUMMARY OF THE INVENTION

The present disclosure provides a compound represented by formula (P) or a pharmaceutically acceptable salt thereof,

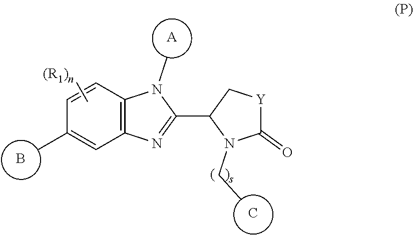

wherein

R₁ is selected from H, F, Cl, Br, I and C₁₋₃ alkyl, wherein the C₁₋₃ alkyl is optionally substituted by 1, 2 or 3 Rₐ;

n is selected from 1, 2 and 3;

s is selected from 0, 1 and 2;

Y is selected from —CH₂O—, —CH₂CH₂—, —N(R_{b})—, —CH₂S— and -cyclopropylidene-;

ring A is selected from cyclohexyl,

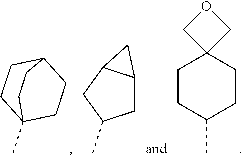

wherein the cyclohexyl,

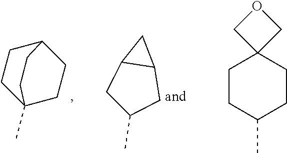

are optionally substituted by 1, 2 or 3 R₂;

ring B is selected from

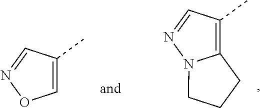

wherein the

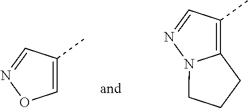

are optionally substituted by 1, 2 or 3 R₃;

ring C is selected from phenyl and 5-6 membered heteroaryl, wherein the phenyl and 5-6 membered heteroaryl are optionally substituted by 1, 2 or 3 R₄;

R₂, R₃ and R₄ are each independently selected from H, F, Cl, Br, I, OH, COOH, C₁₋₃ alkyl and C₁₋₃ alkoxy, wherein the C₁₋₃ alkyl and C₁₋₃ alkoxy are optionally substituted by 1, 2 or 3 R_{c};

Rₐ and R_{c} are each independently selected from F, Cl, Br, I and OH;

R_{b} is independently selected from H and CH₃;

provided that, when Y is selected from —CH₂CH₂— and —CH₂O—, ring A is not cyclohexyl.

In some embodiments of the present disclosure, the above-mentioned R₁ is selected from H, F, Cl, Br, I and CH₃, wherein the CH₃ is optionally substituted by 1, 2 or 3 Rₐ, and other variables are as defined in the present disclosure.

In some embodiments of the present disclosure, the above-mentioned R₁ is selected from H, F, Cl, Br, I, CH₃, CH₂F, CHF₂ and CF₃, and other variables are as defined in the present disclosure.

In some embodiments of the present disclosure, the above-mentioned R₂, R₃ and R₄ are each independently selected from H, F, Cl, Br, I, OH, CH₃ and OCH₃, wherein the CH₃ and OCH₃ are optionally substituted by 1, 2 or 3 R_{c}, and other variables are as defined in the present disclosure.

In some embodiments of the present disclosure, the above-mentioned R₂, R₃ and R₄ are each independently selected from H, F, Cl, Br, I, OH, CH₃, CH₂OH, CH₂F, CHF₂, CF₃ and OCH₃, and other variables are as defined in the present disclosure.

In some embodiments of the present disclosure, the above-mentioned Y is selected from —CH₂O—, —CH₂CH₂—, —NH—, —N(CH₃)—, —CH₂S—,

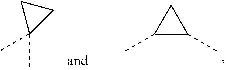

and other variables are as defined in the present disclosure.

In some embodiments of the present disclosure, the above-mentioned ring A is selected from

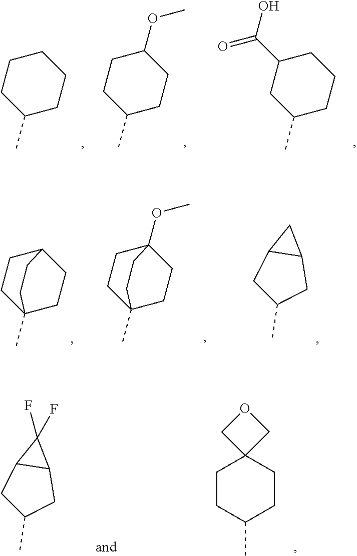

and other variables are as defined in the present disclosure.

In some embodiments of the present disclosure, the above-mentioned ring B is selected from

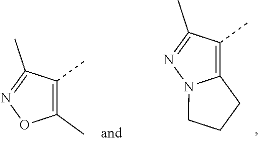

and other variables are as defined in the present disclosure.

In some embodiments of the present disclosure, the above-mentioned ring C is

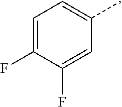

and other variables are as defined in the present disclosure.

In some embodiments of the present disclosure, the above-mentioned ring structural moiety

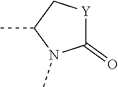

is selected from

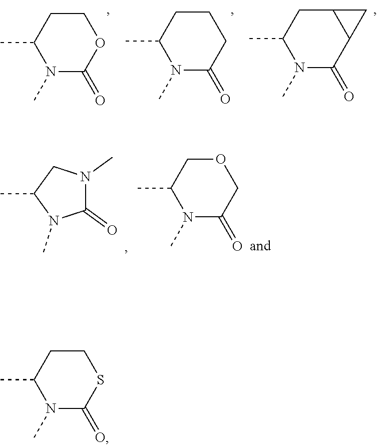

and other variables are as defined in the present disclosure.

The present disclosure provides a compound represented by formula (P-1) or a pharmaceutically acceptable salt thereof,

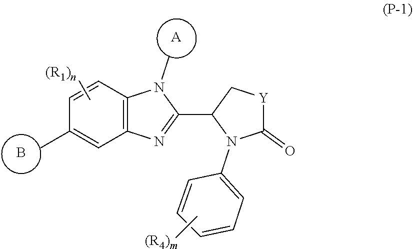

wherein

R₁ is selected from H, F, Cl, Br, I and C₁₋₃ alkyl, wherein the C₁₋₃ alkyl is optionally substituted by 1, 2 or 3 Rₐ;

n is selected from 1, 2 and 3;

R₄ is selected from H, F, Cl, Br, I and C₁₋₃ alkyl, wherein the C₁₋₃ alkyl is optionally substituted by 1, 2 or 3 Rₐ;

m is selected from 1, 2, 3 and 4;

Y is selected from —CH₂O—, —CH₂CH₂—, —N(R_{b})—, —CH₂N(R_{b})—, —CH₂S— and -cyclopropylidene-;

ring A is selected from

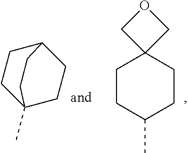

wherein the

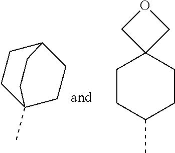

is optionally substituted by 1, 2 or 3 R₂;

ring B is selected from

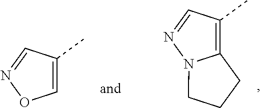

wherein the

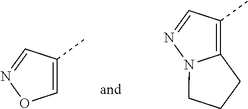

are optionally substituted by 1, 2 or 3 R₃;

R₂ and R₃ are each independently selected from H, F, Cl, Br, I, OH, COOH, C₁₋₃ alkyl and C₁₋₃ alkoxy, wherein the C₁₋₃ alkyl and C₁₋₃ alkoxy are optionally substituted by 1, 2 or 3 R_{c};

Rₐ and R_{c} are each independently selected from F, Cl, Br, I and OH;

R_{b} is independently selected from H and CH₃;

In some embodiments of the present disclosure, the above-mentioned R₁ is selected from H, F, Cl and CH₃, wherein the CH₃ is optionally substituted by 1, 2 or 3 Rₐ, and other variables are as defined in the present disclosure.

In some embodiments of the present disclosure, the above-mentioned R₁ is selected from H, F, Cl, CH₃, CH₂F, CHF₂ and CF₃, and other variables are as defined in the present disclosure.

In some embodiments of the present disclosure, the above-mentioned R₄ is selected from H, F, Cl and CH₃, wherein the CH₃ is optionally substituted by 1, 2 or 3 Rₐ , and other variables are as defined in the present disclosure.

In some embodiments of the present disclosure, the above-mentioned R₄ is selected from H, F, Cl, CH₃, CH₂F, CHF₂ and CF₃, and other variables are as defined in the present disclosure.

In some embodiments of the present disclosure, the above-mentioned R₂ and R₃ are each independently selected from H, F, Cl, Br, I, OH, CH₃ and OCH₃, wherein the CH₃ and OCH₃ are optionally substituted by 1, 2 or 3 R_{c}, and other variables are as defined in the present disclosure.

In some embodiments of the present disclosure, the above-mentioned R₂ and R₃ are each independently selected from H, F, Cl, Br, I, OH, CH₃, CH₂OH, CH₂F, CHF₂, CF₃ and OCH₃, and other variables are as defined in the present disclosure.

In some embodiments of the present disclosure, the above-mentioned Y is selected from —CH₂O—, —CH₂CH₂—, —NH—, —N(CH₃)—, —CH₂NH—, —CH₂N(CH₃)—, —CH₂S—,

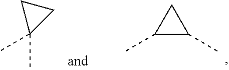

and other variables are as defined in the present disclosure.

In some embodiments of the present disclosure, the above-mentioned ring A is selected from

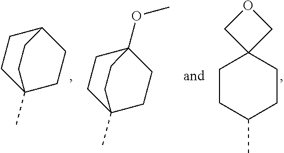

and other variables are as defined in the present disclosure.

In some embodiments of the present disclosure, the above-mentioned ring B is selected from

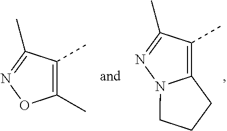

and other variables are as defined in the present disclosure.

In some embodiments of the present disclosure, the above-mentioned ring structural moiety

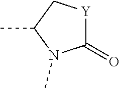

is selected from

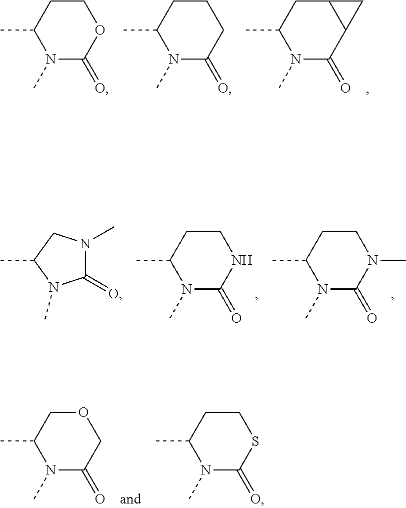

and other variables are as defined in the present disclosure.

The present disclosure provides a compound represented by formula (I) or a pharmaceutically acceptable salt thereof,

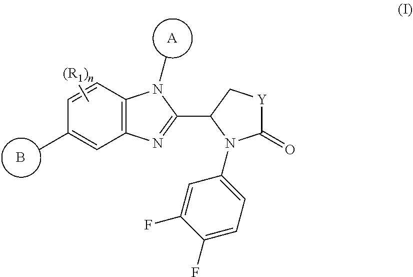

wherein

R₁ is selected from H, F, Cl, Br and C₁₋₃ alkyl, wherein the C₁₋₃ alkyl is optionally substituted by 1, 2 or 3 Rₐ;

n is selected from 1, 2 and 3;

Y is selected from —CH₂O—, —CH₂CH₂—, —N(R_{b})—, —CH₂N(R_{b})—, —CH₂S— and -cyclopropylidene-;

ring A is selected from cyclohexyl,

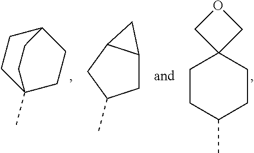

wherein the cyclohexyl,

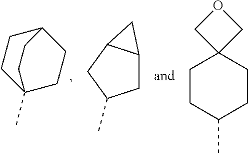

are optionally substituted by 1, 2 or 3 R₂;

ring B is selected from

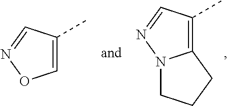

wherein the

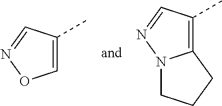

are optionally substituted by 1, 2 or 3 R₃;

R₂ and R₃ are each independently selected from H, F, Cl, Br, I, OH, COOH, C₁₋₃ alkyl and C₁₋₃ alkoxy, wherein the C₁₋₃ alkyl and C₁₋₃ alkoxy are optionally substituted by 1, 2 or 3 R_{c};

Rₐ and R_{c} are each independently selected from F, Cl, Br and OH;

R_{b} is independently selected from H and CH₃;

provided that, when Y is —CH₂CH₂—, ring A is not cyclohexyl.

In some embodiments of the present disclosure, the above-mentioned R₁ is selected from H, F, Cl, Br and CH₃, wherein the CH₃ is optionally substituted by 1, 2 or 3 Rₐ , and other variables are as defined in the present disclosure.

In some embodiments of the present disclosure, the above-mentioned R₁ is selected from H, F, Cl, Br, CH₃, CH₂F, CHF₂ and CF₃, and other variables are as defined in the present disclosure.

In some embodiments of the present disclosure, the above-mentioned R₂ and R₃ are each independently selected from H, F, Cl, Br, I, OH, CH₃ and OCH₃, wherein the CH₃ and OCH₃ are optionally substituted by 1, 2 or 3 R_{c}, and other variables are as defined in the present disclosure.

In some embodiments of the present disclosure, the above-mentioned R₂ and R₃ are each independently selected from H, F, Cl, Br, I, OH, CH₃, CH₂OH, CH₂F, CHF₂, CF₃ and OCH₃, and other variables are as defined in the present disclosure.

In some embodiments of the present disclosure, the above-mentioned Y is selected from —CH₂O—, —CH₂CH₂—, —NH—, —N(CH₃)—, —CH₂NH—, —CH₂N(CH₃)—, —CH₂S—,

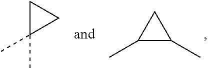

and other variables are as defined in the present disclosure.

In some embodiments of the present disclosure, the above-mentioned ring A is selected from

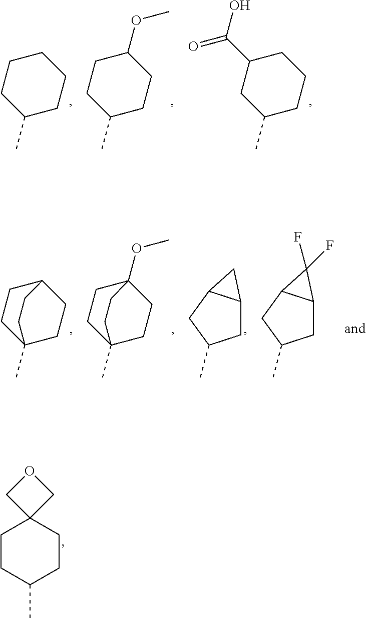

and other variables are as defined in the present disclosure.

In some embodiments of the present disclosure, the above-mentioned ring B is selected from

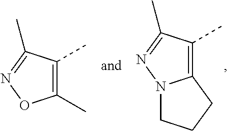

and other variables are as defined in the present disclosure.

In some embodiments of the present disclosure, the above-mentioned ring structural moiety

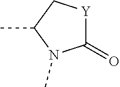

is selected from

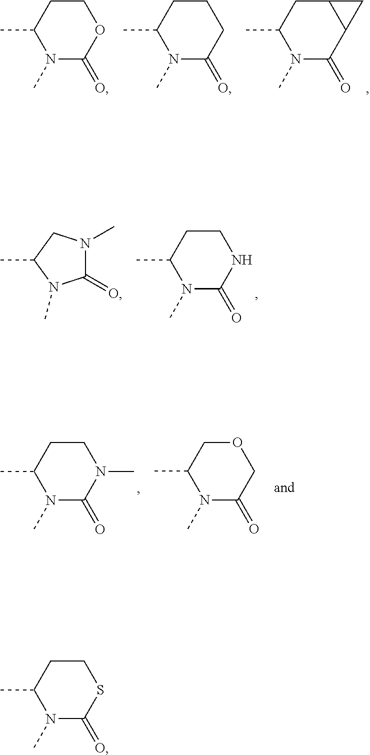

and other variables are as defined in the present disclosure.

The present disclosure also includes some embodiments that are obtained by combining any of the above-mentioned variables.

In some embodiments of the present disclosure, the above-mentioned compound or a pharmaceutically acceptable salt thereof is disclosed, wherein the compound is selected from

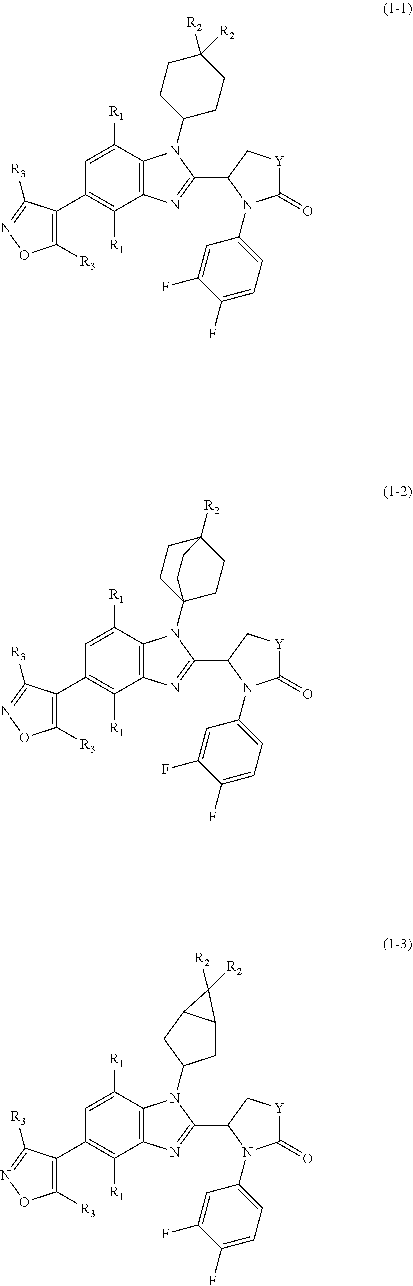

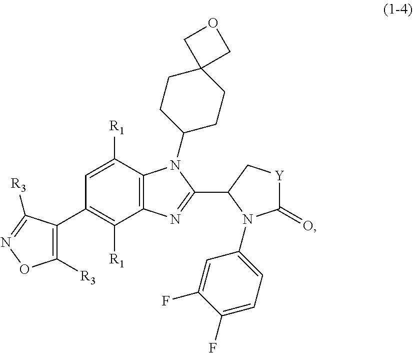

wherein R₁, R₂, R₃ and Y are as defined in the present disclosure.

The present disclosure also provides a compound represented by the following formula or a pharmaceutically acceptable salt thereof,

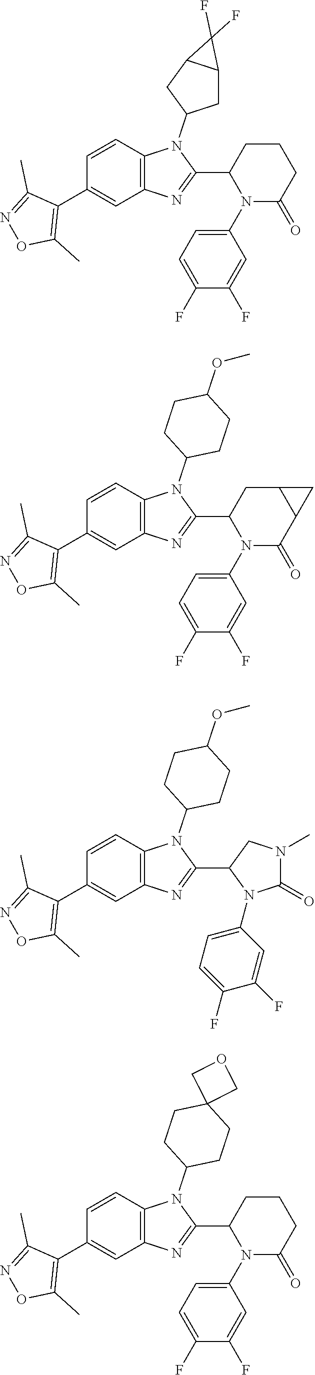

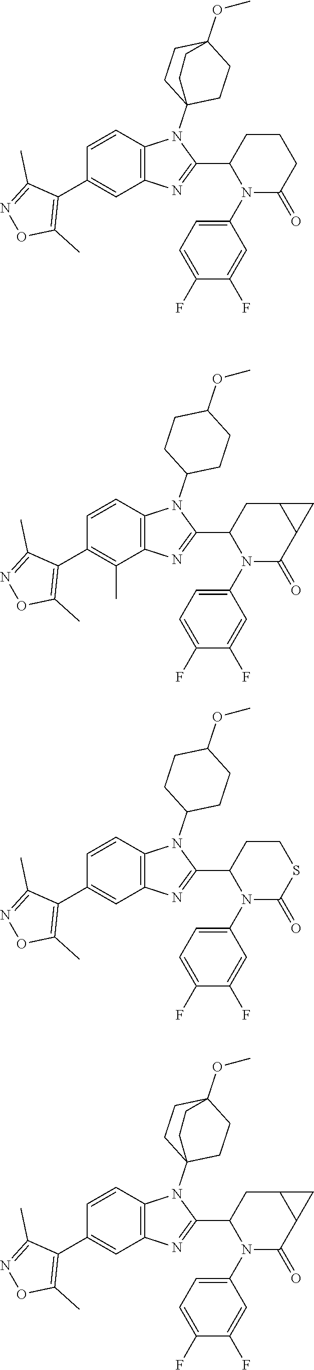

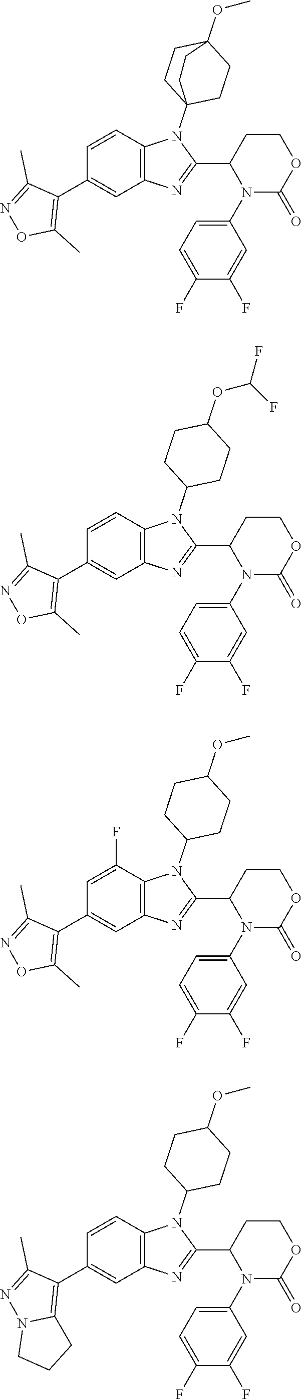

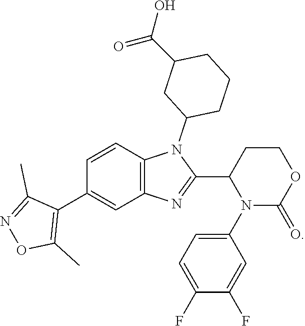 [SMILES: Cc1noc(C)c1-c1ccc2c(c1)nc(C1CCOC(=O)N1c1ccc(F)c(F)c1)n2C1CCCC(C(=O)O)C1]

In some embodiments of the present disclosure, the above-mentioned compound or a pharmaceutically acceptable salt thereof is disclosed, wherein the compound is selected from:

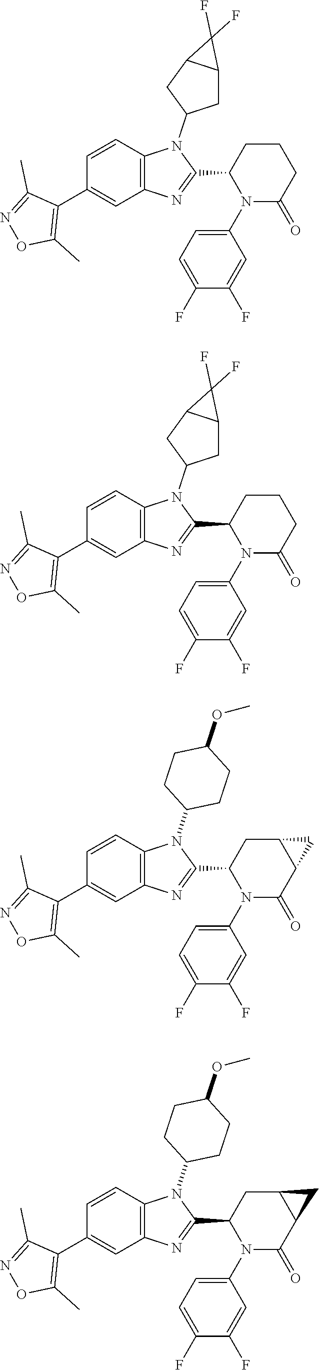

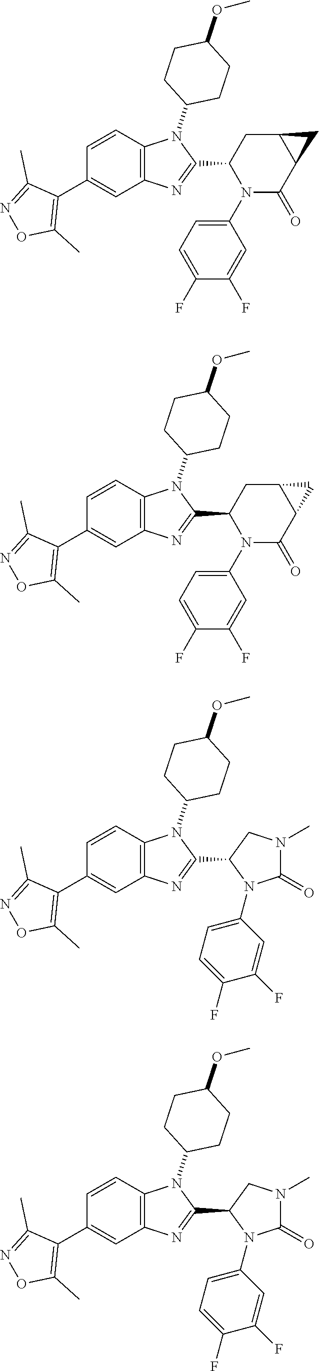

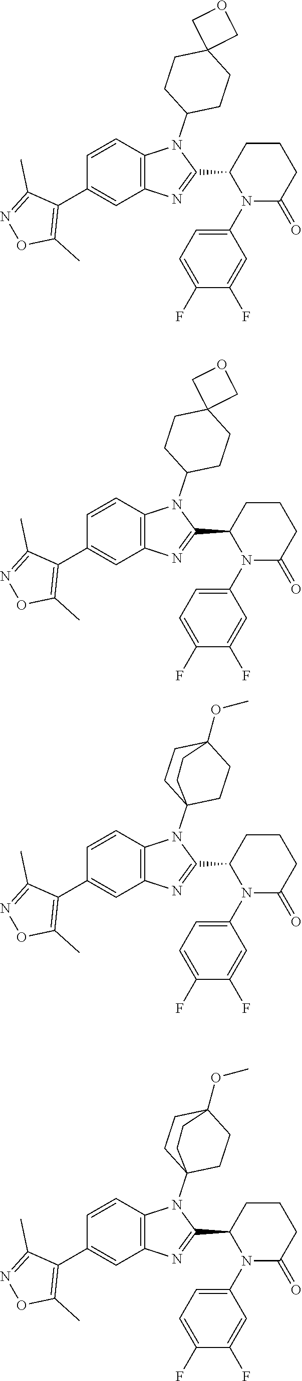

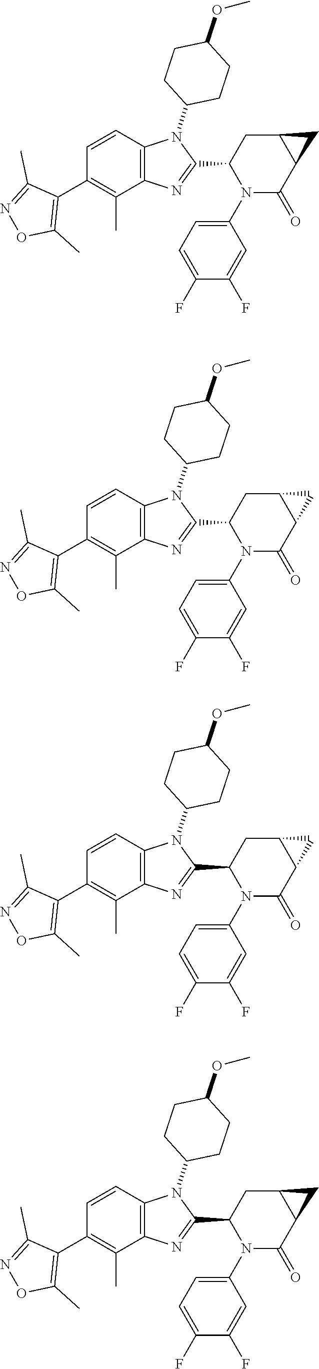

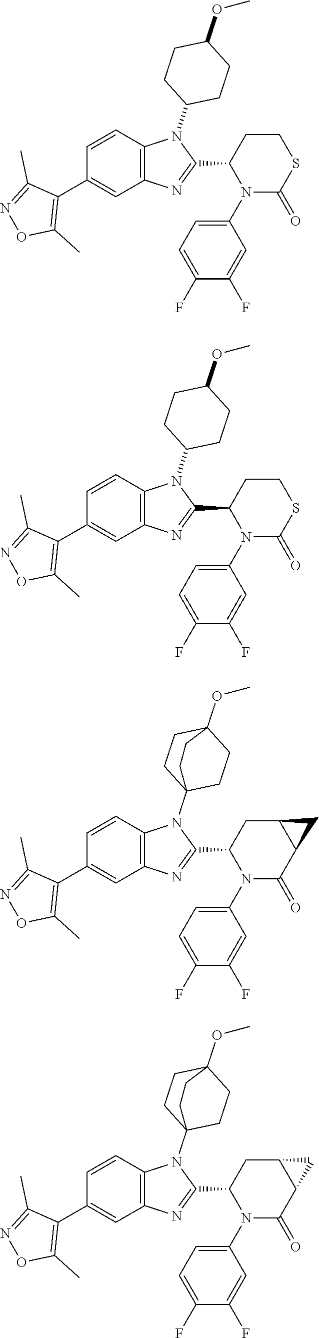

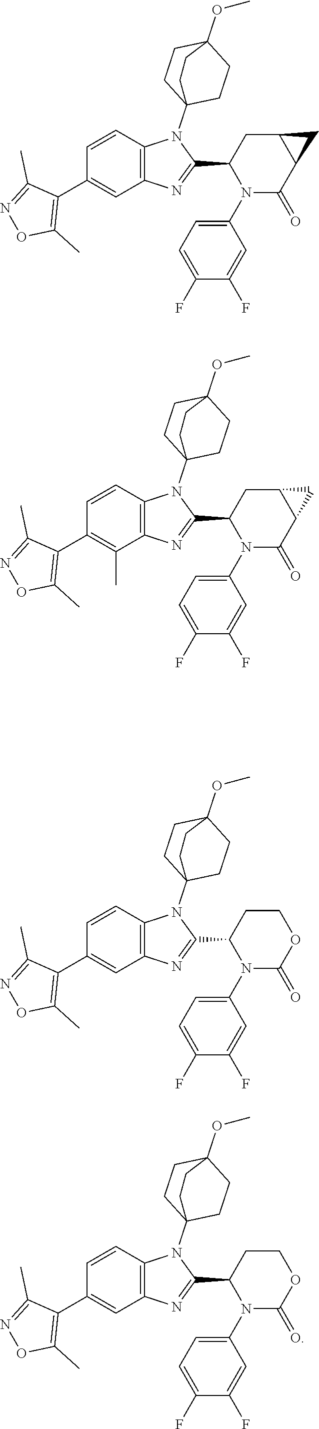

### Technical Effect

The compounds of the present disclosure, as a class of highly active p300/CBP inhibitors, have a great application prospect in treating tumors. The compounds of the present disclosure exhibit good inhibitory activity on p300/CBP. The compounds of the present disclosure have short half-life, wide distribution outside plasma, and moderate bioavailability.

### Definition and Term

Unless otherwise specified, the following terms and phrases used herein are intended to have the following meanings. A specific term or phrase should not be considered indefinite or unclear in the absence of a particular definition, but should be understood in the conventional sense. When a trade name appears herein, it is intended to refer to its corresponding commodity or active ingredient thereof.

The term “pharmaceutically acceptable” is used herein in terms of those compounds, materials, compositions, and/or dosage forms, which are suitable for use in contact with human and animal tissues within the scope of reliable medical judgment, with no excessive toxicity, irritation, allergic reaction or other problems or complications, commensurate with a reasonable benefit/risk ratio.

The term “pharmaceutically acceptable salt” means a salt of compounds disclosed herein that is prepared by reacting the compound having a specific substituent disclosed herein with a relatively non-toxic acid or base. When compounds disclosed herein contain a relatively acidic functional group, a base addition salt can be obtained by bringing the compound into contact with a sufficient amount of base in a pure solution or a suitable inert solvent. The pharmaceutically acceptable base addition salt includes a salt of sodium, potassium, calcium, ammonium, organic amine or magnesium or similar salts. When compounds disclosed herein contain a relatively basic functional group, an acid addition salt can be obtained by bringing the compound into contact with a sufficient amount of acid in a pure solution or a suitable inert solvent. Examples of the pharmaceutically acceptable acid addition salt include an inorganic acid salt, wherein the inorganic acid includes, for example, hydrochloric acid, hydrobromic acid, nitric acid, carbonic acid, bicarbonate, phosphoric acid, monohydrogen phosphate, dihydrogen phosphate, sulfuric acid, hydrogen sulfate, hydroiodic acid, phosphorous acid, and the like; and an organic acid salt, wherein the organic acid includes, for example, acetic acid, propionic acid, isobutyric acid, maleic acid, malonic acid, benzoic acid, succinic acid, suberic acid, fumaric acid, lactic acid, mandelic acid, phthalic acid, benzenesulfonic acid, p-toluenesulfonic acid, citric acid, tartaric acid, and methanesulfonic acid, and the like; and an salt of amino acid (such as arginine and the like), and a salt of an organic acid such as glucuronic acid and the like. Certain specific compounds disclosed herein contain both basic and acidic functional groups and can be converted to any base or acid addition salt.

The pharmaceutically acceptable salt disclosed herein can be prepared from the parent compound that contains an acidic or basic moiety by conventional chemical methods. Generally, such salt can be prepared by reacting the free acid or base form of the compound with a stoichiometric amount of an appropriate base or acid in water or an organic solvent or a mixture thereof.

Unless otherwise specified, the term “isomer” is intended to include geometric isomers, cis- or trans-isomers, stereoisomers, enantiomers, optical isomers, diastereomers, and tautomers.

Compounds disclosed herein may be present in a specific geometric or stereoisomeric form. The present disclosure contemplates all such compounds, including cis and trans isomers, (−)- and (+)-enantiomers, (R)- and (S)-enantiomers, diastereoisomer, (D)-isomer, (L)-isomer, and a racemic mixture and other mixtures, for example, a mixture enriched in enantiomer or diastereoisomer, all of which are encompassed within the scope disclosed herein. The substituent such as alkyl may have an additional asymmetric carbon atom. All these isomers and mixtures thereof are encompassed within the scope disclosed herein.

Unless otherwise specified, the term “enantiomer” or “optical isomer” means stereoisomers that are in a mirrored relationship with each other.

Unless otherwise specified, the term “cis-trans isomer” or “geometric isomer” is produced by the inability of a double bond or a single bond between ring-forming carbon atoms to rotate freely.

Unless otherwise specified, the term “diastereomer” means a stereoisomer in which two or more chiral centers of are contained in a molecule and is in a non-mirrored relationship between molecules.

Unless otherwise specified, “(+)” means dextroisomer, “(−)” means levoisomer, and “(±)” means racemate.

Unless otherwise specified, a wedged solid bond ( [symbol] ) and a wedged dashed bond ( [symbol] ) indicate the absolute configuration of a stereocenter; a straight solid bond ( [symbol] ) and a straight dashed bond ( [symbol] ) indicate the relative configuration of a stereocenter; a wavy line ( [symbol] ) indicates a wedged solid bond ( [symbol] ) or a wedged dashed bond ( [symbol] ); or a wavy line ( [symbol] ) indicates a straight solid bond ( [symbol] ) and a straight dashed bond ( [symbol] ).

Unless otherwise specified, the term “enriched in one isomer”, “isomer enriched”, “enriched in one enantiomer” or “enantiomeric enriched” means that the content of one isomer or enantiomer is less than 100%, and the content of the isomer or enantiomer is 60% or more, or 70% or more, or 80% or more, or 90% or more, or 95% or more, or 96% or more, or 97% or more, or 98% or more, or 99% or more, or 99.5% or more, or 99.6% or more, or 99.7% or more, or 99.8% or more, or 99.9% or more.

Unless otherwise specified, the term “isomer excess” or “enantiomeric excess” means the difference between the relative percentages of two isomers or two enantiomers. For example, if one isomer or enantiomer is present in an amount of 90% and the other isomer or enantiomer is present in an amount of 10%, the isomer or enantiomeric excess (ee value) is 80%.

Optically active (R)- and (S)-isomer, or D and L isomer can be prepared using chiral synthesis or chiral reagents or other conventional techniques. If one kind of enantiomer of certain compound disclosed herein is to be obtained, the pure desired enantiomer can be obtained by asymmetric synthesis or derivative action of chiral auxiliary followed by separating the resulting diastereomeric mixture and cleaving the auxiliary group. Alternatively, when the molecule contains a basic functional group (such as amino) or an acidic functional group (such as carboxyl), the compound reacts with an appropriate optically active acid or base to form a salt of the diastereomeric isomer which is then subjected to diastereomeric resolution through the conventional method in the art to afford the pure enantiomer. In addition, the enantiomer and the diastereoisomer are generally isolated through chromatography which uses a chiral stationary phase and optionally combines with a chemical derivative method (for example, carbamate generated from amine).

Compounds disclosed herein may contain an unnatural proportion of atomic isotopes at one or more of the atoms that make up the compounds. For example, a compound may be labeled with a radioisotope such as tritium (³H), iodine-125 (¹²⁵ I) or C-14 (¹⁴C). For another example, hydrogen can be replaced by heavy hydrogen to form a deuterated drug. The bond between deuterium and carbon is stronger than that between ordinary hydrogen and carbon. Compared with undeuterated drugs, deuterated drugs have advantages of reduced toxic side effects, increased drug stability, enhanced efficacy, and prolonged biological half-life of drugs. All changes in the isotopic composition of compounds disclosed herein, regardless of radioactivity, are included within the scope of the present disclosure.

The term “optional” or “optionally” means that the subsequent event or condition may occur but not requisite, that the term includes the instance in which the event or condition occurs and the instance in which the event or condition does not occur.

The term “substituted” means one or more than one hydrogen atom(s) on a specific atom are substituted by a substituent, and the substituent includes deuterium and hydrogen variants, as long as the valence of the specific atom is normal and the substituted compound is stable. When the substituent is oxo (i.e., ═O), it means two hydrogen atoms are substituted. Positions on an aromatic ring cannot be substituted by oxo. The term “optionally substituted” means an atom can be substituted by a substituent or not, unless otherwise specified, the species and number of the substituent may be arbitrary so long as being chemically achievable.

When any variable (such as R) occurs in the constitution or structure of the compound more than once, the definition of the variable at each occurrence is independent. Thus, for example, if a group is substituted by 0-2 R, the group can be optionally substituted by up to two R, wherein the definition of R at each occurrence is independent. Moreover, a combination of the substituent and/or the variant thereof is allowed only when the combination results in a stable compound.

When the number of a linking group is 0, such as —(CRR)₀—, it means that the linking group is a single bond.

When the number of a substituent is 0, it means that the substituent does not exist. For example, -A-(R)₀ means that the structure is actually -A.

When a substituent is vacant, it means that the substituent does not exist. For example, when X is vacant in A-X, the structure of A-X is actually A.

When one of variables is a single bond, it means that the two groups linked by the single bond are connected directly. For example, when L in A-L-Z represents a single bond, the structure of A-L-Z is actually A-Z.

When the bond of a substituent can be cross-linked to two or more atoms on a ring, such substituent can be bonded to any atom on the ring. For example, a structural moiety

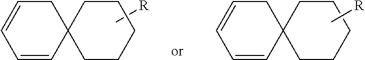

represents the substituent R thereof can be substituted at any site on cyclohexyl or cyclohexadiene. When an enumerated substituent does not indicate through which atom it is linked to the substituted group, such substituent can be bonded through any of its atoms. For example, a pyridyl group as a substituent may be linked to the substituted group through any one of carbon atoms on the pyridine ring.

When an enumerated linking group does not indicate its linking direction, its linking direction is arbitrary. For example, when the linking group L in

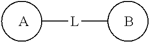

is -M-W-, the -M-W- can be linked to the ring A and the ring B in the same direction as the reading order from left to right to constitute

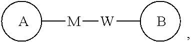

or can be linked to the ring A and the ring B in the reverse direction as the reading order from left to right to constitute

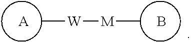

A combination of the linking groups, substituents and/or variants thereof is allowed only when such combination can result in a stable compound.

Unless otherwise specified, when a group has one or more connectable sites, any one or more sites of the group can be connected to other groups through chemical bonds. Where the connection position of the chemical bond is variable, and there is H atom(s) at a connectable site(s), when the connectable site(s) having H atom(s) is connected to the chemical bond, the number of H atom(s) at this site will correspondingly decrease as the number of the connected chemical bond increases, and the group will become a group of corresponding valence. The chemical bond between the site and other groups can be represented by a straight solid bond ( [symbol] ), a straight dashed bond ( [symbol] ), or a wavy line

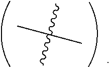

For example, the straight solid bond in —OCH₃ indicates that the group is connected to other groups through the oxygen atom in the group; the straight dashed bond in

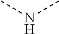

indicates that the group is connected to other groups through two ends of the nitrogen atom in the group; the wavy line in

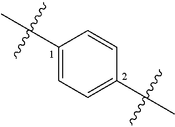 [SMILES: *c1ccc(*)cc1]

indicates that the group is connected to other groups through the 1- and 2-carbon atoms in the phenyl group;

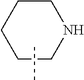

indicates that any connectable site on the piperidinyl group can be connected to other groups through one chemical bond, including at least four connection ways,

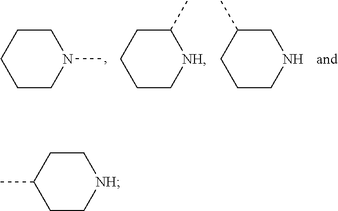

even if a H atom is drawn on —N—,

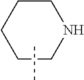

still includes the connection way of

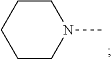

it's just that when one chemical bond is connected, the H at this site will be reduced by one, and the group will become the corresponding monovalent piperidinyl group.

Unless otherwise specified, the number of atoms on a ring is generally defined as the number of ring members, e.g., “5-7 membered ring” refers to a “ring” of 5-7 atoms arranged circumferentially.

Unless otherwise specified, the term “C₁₋₃ alkyl” is used to indicate a linear or branched saturated hydrocarbon group consisting of 1 to 3 carbon atoms. The C₁₋₃ alkyl group includes C₁₋₂ and C₂₋₃ alkyl groups and the like. It may be monovalent (e.g., methyl), divalent (e.g., methylene) or multivalent (e.g., methenyl). Examples of C₁₋₃ alkyl groups include, but are not limited to, methyl (Me), ethyl (Et), propyl (including n-propyl and isopropyl), and the like.

Unless otherwise specified, the term “C₁₋₃ alkoxy” denotes those alkyl groups containing 1 to 3 carbon atoms attached to the rest of the molecule through an oxygen atom. The C₁₋₃ alkoxy group includes C₁₋₂, C₂₋₃, C₃ and C₂ alkoxy groups and the like. Examples of C₁₋₃ alkoxy include, but are not limited to, methoxy, ethoxy, propoxy (including n-propoxy and isopropoxy), and the like.

Unless otherwise specified, the terms “5-6 membered heteroaromatic ring” and “5-6 membered heteroaryl” of the present disclosure may be used interchangeably. The term “5-6 membered heteroaryl” means a monocyclic group having a conjugated 7C electron system and composed of 5 to 6 ring atoms, in which 1, 2, 3 or 4 ring atoms are heteroatoms independently selected from O, S and N, and the remainder is carbon atoms, wherein the nitrogen atom is optionally quaternized and the nitrogen and sulfur heteroatoms are optionally oxidized (i.e., NO and S(O)ₚ, wherein p is 1 or 2). A 5-6 membered heteroaryl can be attached to the remainder of the molecule through a heteroatom or a carbon atom. The 5-6 membered heteroaryl group includes 5-membered and 6-membered heteroaryl groups. Examples of the 5-6 membered heteroaryl include, but are not limited to, pyrrolyl (including N-pyrrolyl, 2-pyrrolyl, 3-pyrrolyl, etc.), pyrazolyl (including 2-pyrazolyl and 3-pyrazolyl, etc.), imidazolyl (including N-imidazolyl, 2-imidazolyl, 4-imidazolyl, and 5-imidazolyl, etc.), oxazolyl (including 2-oxazolyl, 4-oxazolyl, and 5-oxazolyl, etc.), triazolyl (1H-1,2,3-triazolyl, 2H-1,2,3-triazolyl, 1H-1,2,4-triazolyl and 4H-1,2,4-triazolyl, etc.), tetrazolyl, isoxazolyl (3-isoxazolyl, 4-isoxazolyl and 5-isoxazolyl, etc.), thiazolyl (including 2-thiazolyl, 4-thiazolyl and 5-thiazolyl, etc.), furyl (including 2-furyl and 3-furyl, etc.), thienyl (including 2-thienyl and 3-thienyl, etc.), pyridyl (including 2-pyridyl, 3-pyridyl and 4-pyridyl, etc.), pyrazinyl or pyrimidinyl (including 2-pyrimidinyl and 4-pyrimidinyl, etc.).

Compounds disclosed herein can be prepared by a variety of synthetic methods well known to those skilled in the art, including the following enumerated embodiment, the embodiment formed by the following enumerated embodiment in combination with other chemical synthesis methods, and equivalent replacement well known to those skilled in the art. Alternative embodiments include, but are not limited to the examples disclosed herein.

Solvents used in the present disclosure are commercially available.

The following abbreviations are used in the present disclosure: aq represents aqueous; eq represents equivalent or equivalence; DCM represents dichloromethane; PE represents petroleum ether; DMSO represents dimethyl sulfoxide; EtOAc represents ethyl acetate; EtOH represents ethanol; MeOH represents methanol; DMF represents N,N-dimethylformamide; Cbz represents benzyloxycarbonyl, which is an amine protecting group; BOC represents tert-butoxycarbonyl, which is an amine protecting group; r.t. represents room temperature; HATU represents O-(7-azabenzotriazol-1-yl)-N,N,N′,N′-tetramethyluronium hexafluorophosphate; CbzCl represents benzyl chloroformate; DBU represents 1,8-diazabicyclo[5,4,0]undec-7-ene; Pd(dppf)Cl₂ represents [1,1′-bis(diphenylphosphino)ferrocene]palladium dichloride; CU-TMEDA CATALYST(II) represents di-μ-hydroxo-bis[(N,N,N′,N′-tetramethylethylenediamine)copper(II)] chloride.

Compounds are named according to general naming principles in the art or by ChemDraw® software, and commercially available compounds are named with their vendor directory names.

### DETAILED DESCRIPTION OF THE INVENTION

The present disclosure is described in detail below by means of examples. However, it is not intended that these examples have any disadvantageous limitations to the present disclosure. The present disclosure has been described in detail herein, and embodiments are also disclosed herein. It will be apparent to those skilled in the art that various changes and modifications may be made to the embodiments disclosed herein without departing from the spirit and scope disclosed herein.

### EXAMPLE 1

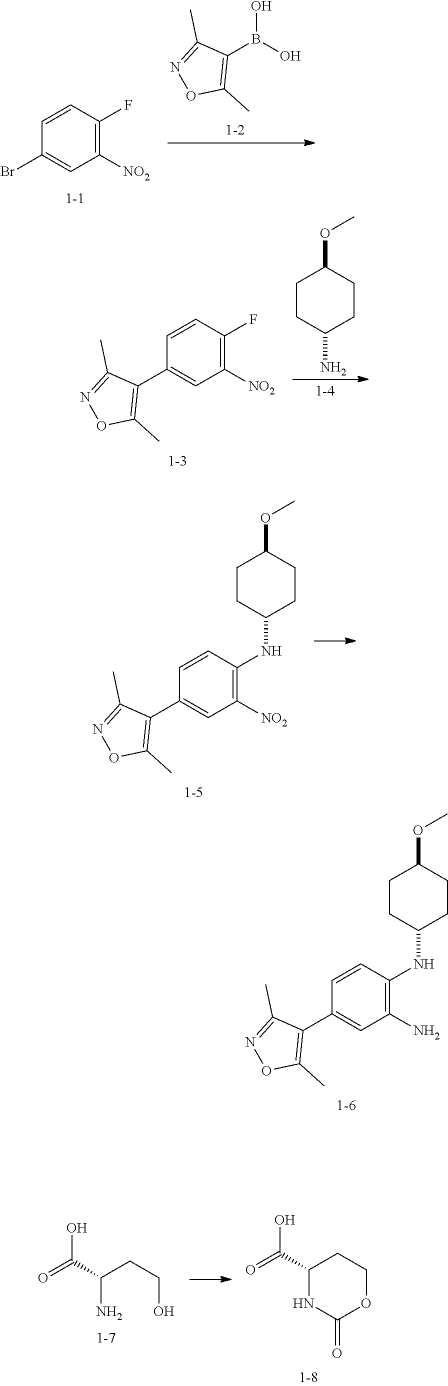

### Preparation of Compound 1-3

To a mixture of 1-1 (5.0 g, 22.73 mmol, 2.79 mL, 1 eq), 1-2 (4.80 g, 34.06 mmol, 1.5 eq), potassium carbonate (9.42 g, 68.18 mmol, 3.0 eq), 1,4-dioxane (60 mL) and water (12 mL) was added Pd(dppf)Cl₂ (1.66 g, 2.27 mmol, 0.1 eq). The atmosphere was replaced with nitrogen three times, and the mixture was heated to 80° C. and stirred for 16 hours. The reaction solution was filtered through a pad of Celite, and the filtrate was concentrated under reduced pressure. To the residue were added ethyl acetate (150 mL) and saturated brine (100 mL) for extraction, and the layers were separated. The organic phase was dried over anhydrous sodium sulfate, and filtered. The filtrate was concentrated under reduced pressure. The residue was purified by column chromatography (petroleum ether/ethyl acetate=5/1˜1/1) to give compound 1-3. LCMS: MS (ESI) m/z (M+H)+: 236.9.

### Preparation of Compound 1-5

To a solution of 1-3 (5.2 g, 22.02 mmol, 1 eq) in tetrahydrofuran (60 mL) were added triethylamine (6.68 g, 66.05 mmol, 9.19 mL, 3 eq) and 1-4 (3.70 g, 28.62 mmol, 1.3 eq), and the resulting reaction solution was heated to 70° C. and stirred for 16 hours. The reaction solution was poured into water (150 mL), and the mixture was extracted with ethyl acetate (100 mL*3). The organic phases were combined, washed with saturated brine (150 mL), dried over anhydrous sodium sulfate, and filtered. The filtrate was concentrated under reduced pressure, and the residue was purified by column chromatography (petroleum ether/ethyl acetate=3/1˜1/1) to give compound 1-5. LCMS: MS (ESI) m/z (M+H)+: 346.1.

### Preparation of Compound 1-6

To a solution of sodium hydrosulfite (17.14 g, 98.42 mmol, 10 eq), aqueous ammonia (24.66 g, 197.03 mmol, 27.1 mL, 28% purity, 20.02 eq), tetrahydrofuran (50 mL) and water (50 mL) was added compound 1-5 (3.4 g, 9.84 mmol, 1 eq), and the resulting reaction solution was stirred at 15° C. for 2 hours. The reaction solution was poured into water (150 mL), and extracted with ethyl acetate (200 mL*2). The organic phases were combined, washed with saturated brine (150 mL), dried over anhydrous sodium sulfate, and filtered. The filtrate was concentrated to dryness under reduced pressure to give compound 1-6. LCMS: MS (ESI) m/z (M+H)+: 316.0.

### Preparation of Compound 1-8

To a mixture of compound 1-7 (1.0 g, 8.39 mmol, 1 eq), sodium hydroxide (2.04 g, 51.00 mmol, 6.08 eq) and water (25.5 mL) at 0° C. was added dropwise a solution of triphosgene (2.59 g, 8.73 mmol, 1.04 eq) dissolved in 1,4-dioxane (12.5 mL) (kept at 0˜5° C.), and the resulting reaction solution was slowly warmed to 15° C. and stirred for 48 hours. The reaction solution was concentrated under reduced pressure, and acetonitrile (20 mL) was added. The mixture was heated to 60° C. and stirred for 0.5 hours, and filtered while hot. The filtrate was concentrated to about 10 mL, and a precipitate was precipitated. The mixture was filtered, and the filter cake was collected to give compound 1-8. LCMS: MS (ESI) m/z (M+H)+: 145.8.

### EXAMPLE 2

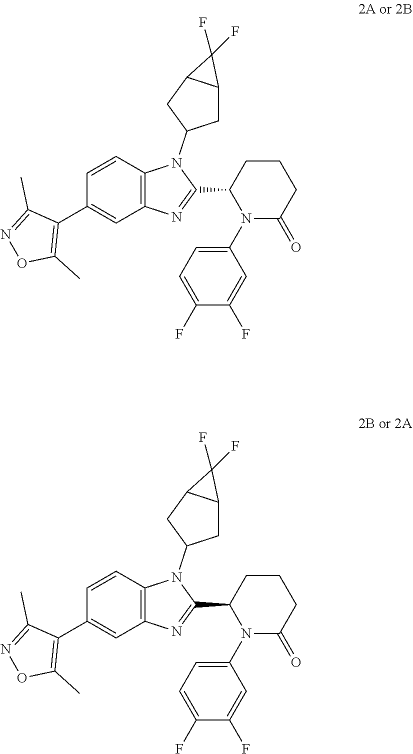 [SMILES: Cc1noc(C)c1-c1ccc2c(c1)nc([C@@H]1CCCC(=O)N1c1ccc(F)c(F)c1)n2C1CC2C(C1)C2(F)F ; Cc1noc(C)c1-c1ccc2c(c1)nc([C@H]1CCCC(=O)N1c1ccc(F)c(F)c1)n2C1CC2C(C1)C2(F)F]

Route of Synthesis:

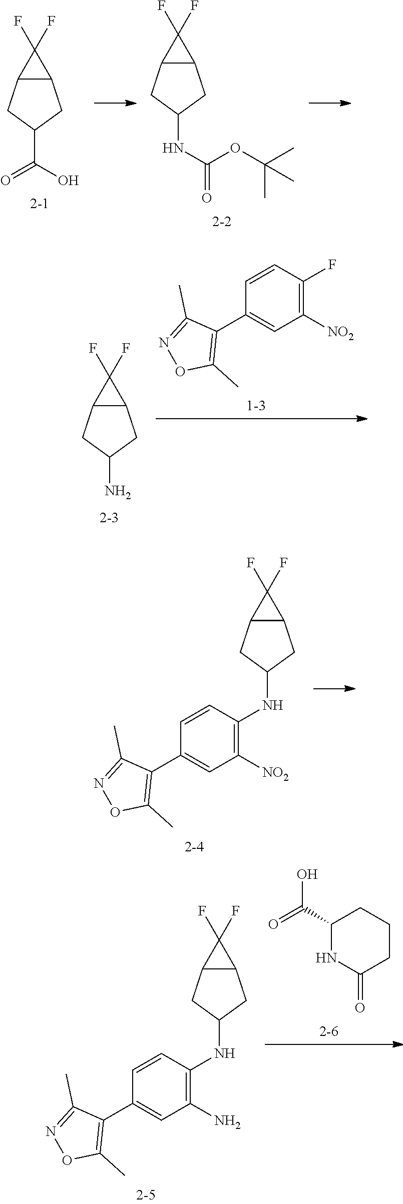

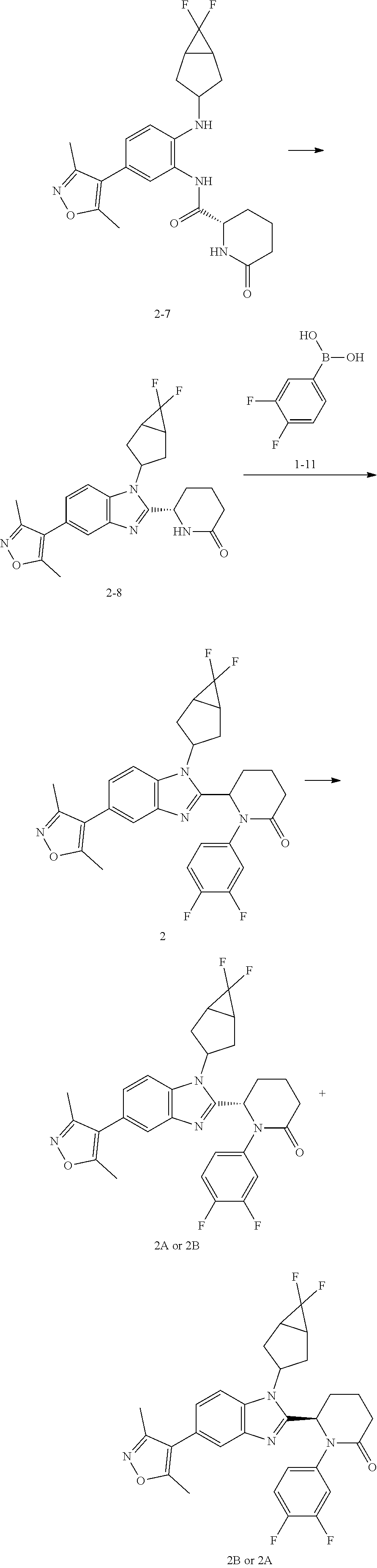 [SMILES: Cc1noc(C)c1-c1ccc(NC2CC3C(C2)C3(F)F)c(NC(=O)[C@@H]2CCCC(=O)N2)c1 ; Cc1noc(C)c1-c1ccc2c(c1)nc([C@@H]1CCCC(=O)N1)n2C1CC2C(C1)C2(F)F ; OB(O)c1ccc(F)c(F)c1 ; Cc1noc(C)c1-c1ccc2c(c1)nc(C1CCCC(=O)N1c1ccc(F)c(F)c1)n2C1CC2C(C1)C2(F)F ; Cc1noc(C)c1-c1ccc2c(c1)nc([C@@H]1CCCC(=O)N1c1ccc(F)c(F)c1)n2C1CC2C(C1)C2(F)F ; Cc1noc(C)c1-c1ccc2c(c1)nc([C@H]1CCCC(=O)N1c1ccc(F)c(F)c1)n2C1CC2C(C1)C2(F)F]

### Preparation of Compound 2-2

To a solution of compound 2-1 (1.0 g, 6.17 mmol, 1 eq) in toluene (25 mL) were added diphenylphosphoryl azide (2.55 g, 9.25 mmol, 2.00 mL, 1.5 eq) and triethylamine (1.25 g, 12.34 mmol, 1.72 mL, 2 eq). The resulting reaction solution was stirred at 15° C. for 0.5 hours and then tert-butanol (5.81 g, 78.42 mmol, 7.5 mL, 12.71 eq) was added. The resulting reaction solution was heated to 100° C. and stirred for 3 hours. To the reaction solution was added ethyl acetate (60 mL). The mixture was washed sequentially with water (50 mL), saturated aqueous sodium bicarbonate solution (30 mL) and saturated brine (50 mL), dried over anhydrous sodium sulfate, and filtered. The filtrate was concentrated under reduced pressure. The residue was purified by column chromatography (petroleum ether/ethyl acetate=1/0˜3/1) to give compound 2-2. ¹H NMR (400 MHz, CDCl₃) δ 4.43-4.24 (m, 1H), 4.04-3.83 (m, 1H), 2.30 (br dd, J=7.5, 13.6 Hz, 2H), 1.91-1.84 (m, 2H), 1.73-1.64 (m, 2H), 1.36 (s, 9H).

### Preparation of Compound 2-3

A mixture of compound 2-2 (0.5 g, 2.14 mmol, 1 eq) and hydrochloric acid/ethyl acetate (4 M, 5 mL, 9.33 eq) was stirred at 20° C. for 16 hours. The reaction solution was concentrated under reduced pressure. To the residue was added petroleum ether/ethyl acetate (10.1 mL, v:v=10:0.1). The mixture was slurried and filtered. The filter cake was collected to give the hydrochloride of compound 2-3. ¹H NMR (400 MHz, DMSO-d₆) δ 8.30 (br s, 3H), 2.68 (br s, 1H), 2.36-2.19 (m, 4H), 2.17-2.04 (m, 2H).

### Preparation of Compound 2-4

To a solution of compound 1-3 (50 mg, 211.69 μmol, 1 eq) in tetrahydrofuran (2 mL) were added triethylamine (65 mg, 642.36 μmol, 89.41 μL, 3.03 eq) and compound 2-3 (47 mg, 277.12 μmol, 1.31 eq, hydrochloride), and the resulting reaction solution was heated to 70° C. and stirred for 16 hours. The reaction solution was poured into water (10 mL), and the mixture was extracted with ethyl acetate (15 mL*3). The organic phases were combined, washed with saturated brine (15 mL), dried over anhydrous sodium sulfate, and filtered. The filtrate was concentrated under reduced pressure, and the residue was purified by column chromatography (petroleum ether/ethyl acetate=3/1˜1/1) to give the compound 2-4. LCMS: MS (ESI) m/z (M+H)+: 350.0.

### Preparation of Compound 2-5

To a solution of sodium hydrosulfite (748 mg, 4.30 mmol, 935.00 μL, 10.01 eq), aqueous ammonia (1.09 g, 8.72 mmol, 1.2 mL, 28% purity, 20.32 eq), tetrahydrofuran (5 mL) and water (5 mL) was added compound 2-4 (150 mg, 429.39 μmol, 1 eq), and the resulting reaction solution was stirred at 20° C. for 2 hours. The reaction solution was poured into water (15 mL), and the mixture was extracted with ethyl acetate (20 mL*2). The organic phase was washed with saturated brine (15 mL), dried over anhydrous sodium sulfate, and filtered. The filtrate was concentrated under reduced pressure to give compound 2-5. LCMS: MS (ESI) m/z (M+H)+: 320.0.

### Preparation of Compound 2-7

To a solution of compound 2-6 (45 mg, 314.38 μmol, 1 eq) in N,N-dimethylformamide (2 mL) were added HATU (132 mg, 347.16 μmol, 1.11 eq), compound 2-5 (100 mg, 313.14 μmol, 1 eq) and triethylamine (95 mg, 938.83 μmol, 130.67 μL, 3 eq), and the resulting reaction solution was stirred at 25° C. for 16 hours. To the reaction solution were added water (20 mL) and ethyl acetate (30 mL), and the layers were separated. The organic phase was washed sequentially with 1N hydrochloric acid solution (20 mL), saturated aqueous sodium bicarbonate solution (20 mL) and saturated brine (20 mL), dried over anhydrous sodium sulfate, and filtered. The filtrate was concentrated under reduced pressure. The residue was purified by column chromatography (dichloromethane/methanol=10/1˜20/1) to give compound 2-7. LCMS: MS (ESI) m/z (M+H)+: 445.1.

### Preparation of Compound 2-8

A solution of compound 2-7 (50 mg, 112.49 μmol, 1 eq) and acetic acid (2 mL) was heated to 80° C. and stirred for 16 hours. The reaction solution was concentrated to dryness under reduced pressure, and dichloromethane (30 mL) was added. The mixture was washed with saturated sodium bicarbonate solution (20 mL), dried over anhydrous sodium sulfate, and filtered. The filtrate was concentrated under reduced pressure to give compound 2-8. LCMS: MS (ESI) m/z (M+H)+: 427.1.

### Preparation of Compound 2A&2B

To a solution of compound 2-8 (50 mg, 117.24 μmol, 1 eq), dichloromethane (1 mL) and acetonitrile (2 mL) was added DBU (40 mg, 262.75 μmol, 39.60 μL, 2.24 eq) at 25° C., and the mixture was stirred for 15 minutes. CU-TMEDA CATALYST (II) (27 mg, 58.14 μmol, 4.96e-1 eq) was added, and the mixture was stirred for 15 minutes. Compound 1-11 (40 mg, 253.31 μmol, 2.16 eq) was added, and the resulting reaction solution was stirred at 25° C. for 48 hours. To the reaction solution were added additional compound 1-11 (40 mg, 253.31 μmol, 2.16 eq) and CU-TMEDA CATALYST (II) (27 mg, 58.14 μmol, 4.96e-1 eq), and the resulting reaction solution was stirred at 25° C. for 16 hours. The reaction solution was filtered, and the filtrate was concentrated under reduced pressure. The residue was purified by column chromatography (dichloromethane/tetrahydrofuran=1/0˜2/1) to give a product. The product was analyzed to be racemic compound 2 (racemization was presumed to have occurred during the preparation of 2-7 or 2-8 or compound 2) by detection with supercritical fluid chromatography (Chiralpak AS-3 100×4.6 mm I.D., 3 μm; mobile phase: A: supercritical carbon dioxide, B: 0.05% diethylamine in ethanol; gradient: from 5% B to 40% B in 4 minutes, 40% B for 2.5 min, from 5% B back to 1.5% B in 1.5 min; flow rate: 2.8 mL/min; column temperature: 35° C.; wavelength: 220 nm). Compound 2 was isolated by SFC (column: DAICEL CHIRALPAK AS (250 mm*30 mm, 10 μm); mobile phase: [0.1% NH₃H₂O EtOH]; 25%-25%) to give chiral isomers compound 2A and compound 2B.

Compound 2A (retention time was 2.256 min, ee=97.84%): ¹H NMR (400 MHz, DMSO-d₆) 67 7.74 (d, J=1.3 Hz, 1H), 7.48 (d, J=8.3 Hz, 1H), 7.43-7.29 (m, 2H), 7.23 (dd, J=1.5, 8.5 Hz, 1H), 7.13-7.06 (m, 1H), 5.72 (t, J=4.5 Hz, 1H), 4.92-4.86 (mz, 1H), 2.73-2.57 (m, 4H), 2.41 (s, 6H), 2.35-2.32 (m, 1H), 2.24 (s, 3H), 2.16-2.00 (m, 2H), 1.92 (br s, 1H), 1.81 (br s, 1H); LCMS: MS (ESI) m/z (M+H)+: 539.4.

Compound 2B (retention time was 3.080 min, ee=99.36%): ¹H NMR (400 MHz, DMSO-d₆) δ 7.80 (d, J=1.3 Hz, 1H), 7.53 (d, J=8.3 Hz, 1H), 7.49-7.35 (m, 2H), 7.29 (dd, J=1.5, 8.5 Hz, 1H), 7.15-7.01 (m, 1H), 5.78 (t, J=4.5 Hz, 1H), 4.91-4.87(m, 1H), 2.77-2.61 (m, 4H), 2.51-2.42 (m, 6H), 2.42-2.37 (m, 1H), 2.30 (s, 3H), 2.21-2.06 (m, 2H), 1.97 (br s, 1H), 1.87 (br s, 1H); LCMS: MS (ESI) m/z (M+H)+: 539.4.

Analysis method: Chiralpak AS-3 100×4.6 mm I.D., 3 μm; mobile phase: A: supercritical carbon dioxide, B: 0.05% diethylamine in ethanol; gradient: from 5% B to 40% B in 4 minutes, 40% B for 2.5 min, from 5% B back to 1.5% B in 1.5 min; flow rate: 2.8 mL/min; column temperature: 35° C.; wavelength: 220 nm.

### EXAMPLE 3

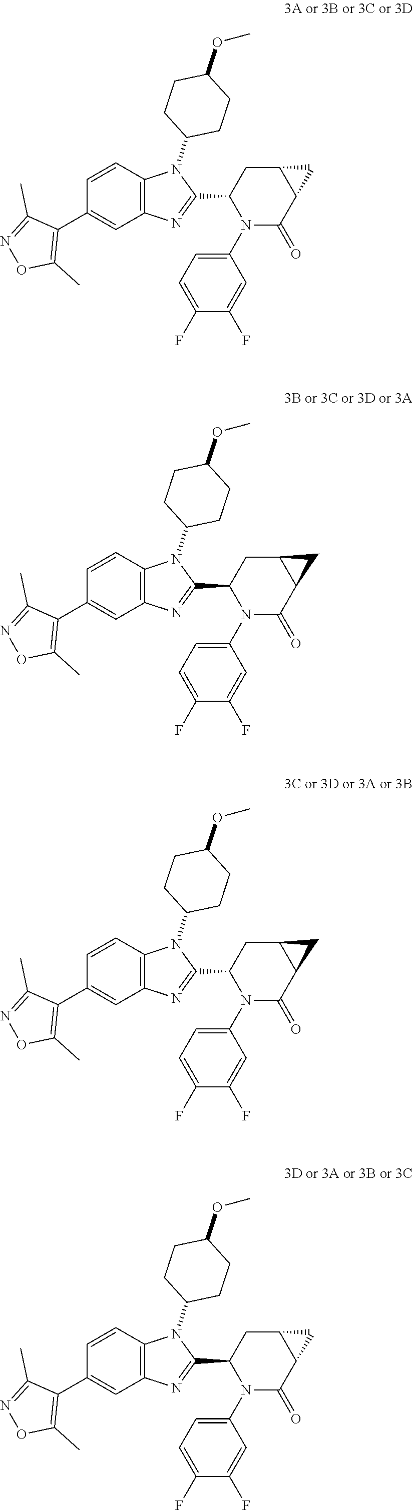

Route of Synthesis:

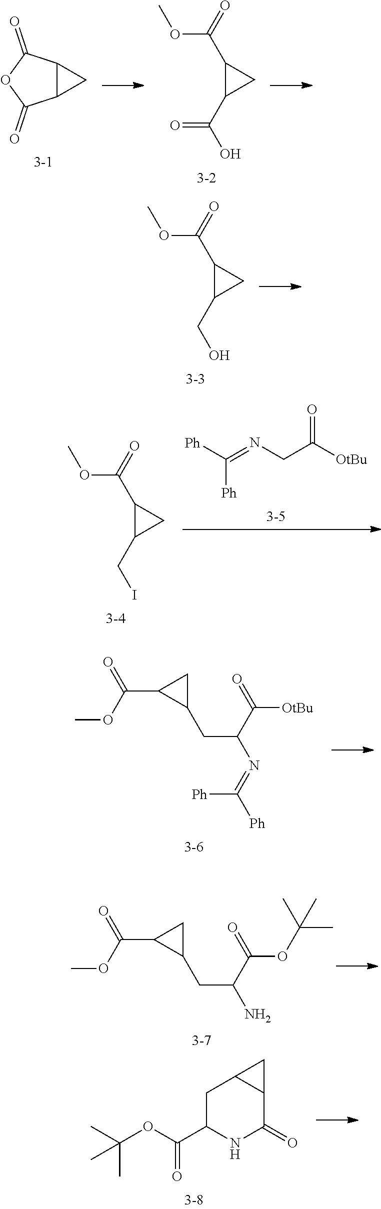

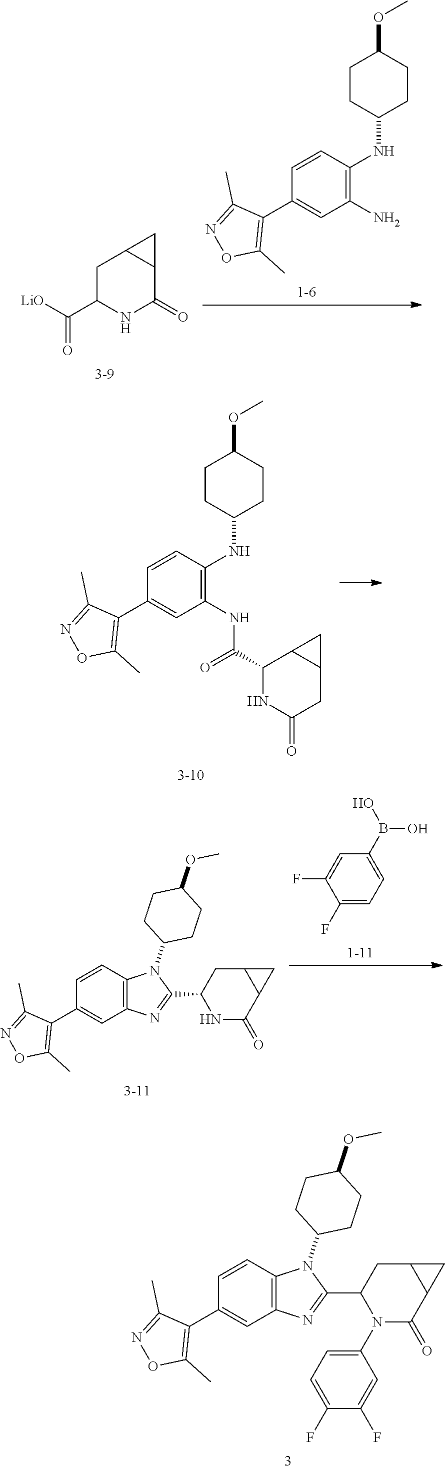

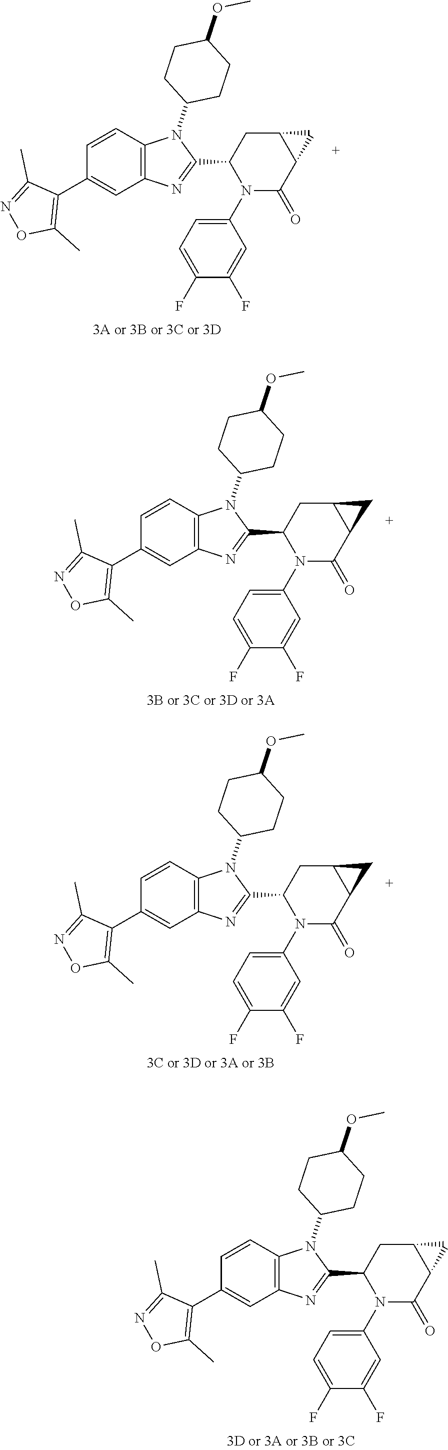

### Preparation of Compound 3-2

To methanol (65 mL) were added compound 3-1 (5.0 g, 44.61 mmol, 1 eq) and triethylamine (4.51 g, 44.61 mmol, 6.21 mL, 1 eq) at 0° C., and the resulting reaction solution was warmed to 20° C. and stirred for 1 hour. The reaction solution was concentrated under reduced pressure, and the residue was dissolved in ethyl acetate (150 mL). The mixture was washed sequentially with 1N HCl solution (50 mL) and saturated brine (50 mL), dried over anhydrous sodium sulfate, and filtered. The filtrate was concentrated under reduced pressure to give compound 3-2. ¹H NMR (400 MHz, CDCl₃) δ 3.72 (s, 3H), 2.19-2.07 (m, 2H), 1.70 (dt, J=5.3, 6.8 Hz, 1H), 1.35 (dt, J=5.1, 8.5 Hz, 1H).

### Preparation of Compound 3-3

To a solution of compound 3-2 (5.9 g, 40.94 mmol, 1 eq) in tetrahydrofuran (20 mL) was added borane-dimethyl sulfide (10 M, 4.91 mL, 1.2 eq) dropwise at 0° C., and the resulting reaction solution was warmed to 20° C. and stirred for 16 hours. The reaction solution was placed in an ice-water bath, and methanol (15 mL) was added dropwise. After the mixture was stirred for 30 minutes, methanol (100 mL) was added and the mixture was concentrated under reduced pressure to give compound 3-3. ¹H NMR (400 MHz, CDCl₃) δ 3.96 (dd, J=5.0, 11.8 Hz, 1H), 3.76 (dd, J=8.0, 11.8 Hz, 1H), 3.71 (s, 3H), 2.20-2.04 (m, 1H), 1.79 (dt, J=5.9, 8.2 Hz, 1H), 1.68-1.56 (m, 1H), 1.20-1.10 (m, 2H).

### Preparation of Compound 3-4

To a solution of compound 3-3 (2.36 g, 18.13 mmol, 1 eq), triphenylphosphine (8.56 g, 32.64 mmol, 1.8 eq), imidazole (2.33 g, 34.29 mmol, 1.89 eq), acetonitrile (30 mL) and tetrahydrofuran (45 mL) was added elementary iodine (9.21 g, 36.27 mmol, 7.31 mL, 2 eq) at 0° C., and the resulting reaction solution was stirred at 0° C. for 2 hours. To the reaction solution was added methyl tert-butyl ether (200 mL). The mixture was washed sequentially with 20% aqueous sodium thiosulfate solution (200 mL*2) and saturated brine (200 mL). The organic phase was dried over anhydrous sodium sulfate, and filtered. The filtrate was concentrated under reduced pressure. To the residue was added n-hexane (100 mL) and the mixture was stirred at room temperature for 1 hour. The mixture was filtered. The filtrate was concentrated under reduced pressure, and the residue was purified by column chromatography (petroleum ether/ethyl acetate=1/0˜5/1) to give compound 3-4. ¹H NMR (400 MHz, CDCl₃) δ 3.76 (s, 3H), 3.58 (dd, J=6.9, 9.9 Hz, 1H), 3.38 (t, J=9.5 Hz, 1H), 1.99 (dt, J=5.8, 8.2 Hz, 1H), 1.93-1.82 (m, 1H), 1.33 (dt, J=5.0, 8.2 Hz, 1H), 1.22-1.13 (m, 1H); LCMS: MS (ESI) m/z (M+H)+: 241.0.

### Preparation of Compound 3-6

To a solution of compound 3-4 (2.35 g, 7.94 mmol, 1 eq) in tetrahydrofuran (20 mL) was added sodium tert-butoxide (900 mg, 8.02 mmol, 1.01 eq) at −78° C. After the mixture was stirred at −78° C. for 0.5 hours, a solution of compound 3-5 (1.9 g, 7.92 mmol, 1 eq) in tetrahydrofuran (10 mL) was added dropwise, and the resulting reaction solution was slowly warmed to room temperature (20° C.) and stirred for 16 hours. The reaction solution was poured into ice water (50 mL), and extracted with methyl tert-butyl ether (50 mL*3). The organic phases were combined, washed with water (50 mL) and saturated brine (50 mL), dried over anhydrous sodium sulfate, and filtered. The filtrate was concentrated under reduced pressure to give compound 3-6. LCMS: MS (ESI) m/z (M+H)+: 408.3.

### Preparation of Compound 3-7

To a solution of compound 3-6 (3.0 g, 7.36 mmol, 1 eq) in tetrahydrofuran (40 mL) was added a solution of citric acid (8.49 g, 44.17 mmol, 8.49 mL, 6 eq) in water (20 mL) at 0° C., and the resulting reaction solution was warmed to 20° C. and stirred for 2 hours. To the reaction solution was added n-hexane (100 mL), and the layers were separated. To the aqueous phase was added saturated sodium bicarbonate solution, and the mixture was extracted with ethyl acetate (100 mL). The organic phase was washed with saturated brine (80 mL), dried over anhydrous sodium sulfate, and filtered. The filtrate was concentrated under reduced pressure to give compound 3-7. ¹H NMR (400 MHz, CDCl₃) δ 3.62 (s, 3H), 3.31-3.24 (m, 1H), 1.95-1.70 (m, 2H), 1.70-1.65 (m, 1H), 1.56-1.51 (m, 2H), 1.40 (d, J=1.0 Hz, 9H), 1.36-1.25 (m, 1H), 1.05-0.99 (m, 1H), 0.96-0.87 (m, 1H); LCMS: MS (ESI) m/z (M+H)+: 244.1.

### Preparation of Compound 3-8

To a solution of compound 3-7 (1.4 g, 5.75 mmol, 1 eq) in toluene (40 mL) was added concentrated hydrochloric acid (56 mg, 568.28 μmol, 55 μL, 37% purity, 9.88e-2 eq), and the resulting reaction solution was heated to 105° C. and stirred for 16 hours. The reaction solution was concentrated under reduced pressure to give compound 3-8. LCMS: MS (ESI) m/z (M+H)+: 212.1.

### Preparation of Compound 3-9

To a solution of compound 3-8 (1.2 g, 5.68 mmol, 1 eq) in tetrahydrofuran (10 mL) and water (10 mL) was added lithium hydroxide monohydrate (480 mg, 11.44 mmol, 2.01 eq), and the resulting reaction solution was stirred at 20° C. for 16 hours. To the reaction solution was added water (30 mL), and the mixture was directly freeze-dried to give compound 3-9. LCMS: MS (ESI) m/z (M+H)+: 156.1.

### Preparation of Compound 3-10

To a solution of compound 3-9 (0.45 g, 2.90 mmol, 3.05 eq) in N,N-dimethylformamide (10 mL) were added HATU (1.20 g, 3.16 mmol, 3.32 eq), compound 1-6 (300 mg, 951.15 μmol, 1 eq) and triethylamine (300 mg, 2.96 mmol, 412.65 μL, 3.12 eq), and the resulting reaction solution was stirred at 30° C. for 16 hours. The reaction solution was poured into water (40 mL). The mixture was extracted with ethyl acetate (50 mL*2), and the layers were separated. The organic phases were combined, washed sequentially with saturated sodium bicarbonate solution (50 mL) and saturated brine (50 mL), dried over anhydrous sodium sulfate, and filtered. The filtrate was concentrated under reduced pressure. The residue was purified by column chromatography (dichloromethane/tetrahydrofuran=1/0˜0/1) to give compound 3-10. LCMS: MS (ESI) m/z (M+H)+: 453.4.

### Preparation of Compound 3-11

A solution of compound 3-10 (100 mg, 220.97 μmol, 1 eq) and acetic acid (2 mL) was heated to 80° C. and stirred for 16 hours. The reaction solution was concentrated under reduced pressure. To the residue was added dichloromethane (30 mL). The mixture was washed with saturated aqueous sodium bicarbonate solution (20 mL), dried over anhydrous sodium sulfate, and filtered. The filtrate was concentrated under reduced pressure. The residue was purified by column chromatography (dichloromethane/tetrahydrofuran=5/1˜0/1) to give compound 3-11. LCMS: MS (ESI) m/z (M+H)+: 435.3.

### Preparation of Compound 3A, Compound 3B, Compound 3C and Compound 3D

To a solution of compound 3-11 (60 mg, 138.08 μmol, 1 eq), dichloromethane (2 mL) and acetonitrile (2 mL) was added DBU (50 mg, 328.44 μmol, 49.50 μL, 2.38 eq), and the mixture was stirred for 15 minutes. CU-TMEDA CATALYST (II) (13 mg, 27.99 μmol, 2.03e-1 eq) was added, and the mixture was stirred for 15 minutes. Compound 1-11 (44 mg, 278.64 μmol, 2.02 eq) was added, and the resulting reaction solution was stirred at 30° C. for 16 hours. To the reaction solution were added additional compound 1-11 (30 mg, 189.98 μmol, 1.38 eq) and CU-TMEDA CATALYST (II) (13 mg, 27.99 μmol, 2.03e-1 eq), and the resulting reaction solution was stirred at 30° C. for another 16 hours. The reaction solution was filtered, and the filtrate was concentrated under reduced pressure. The residue was purified by column chromatography (dichloromethane/tetrahydrofuran=5/1˜0/1) to give a product. The product was analyzed to be racemic compound 3 (racemization was presumed to have occurred during the preparation of 3-10 or 3-11 or compound 3) by detection with supercritical fluid chromatography (Chiralpak AS-3 100×4.6 mm I.D., 3 μm; mobile phase: A: supercritical carbon dioxide, B: 0.05% diethylamine in ethanol; gradient: from 5% B to 40% B in 4 minutes, 40% B for 2.5 min, from 5% B back to 1.5% B in 1.5 min; flow rate: 2.8 mL/min; column temperature: 35° C.; wavelength: 220 nm). Compound 3 was isolated by SFC (column: DAICEL CHIRALPAK AS (250 mm*30 mm,10 μm); mobile phase: [0.1% NH₃H₂O EtOH]; 25%-25%) to give chiral isomers compound 3A, compound 3B, compound 3C and compound 3D.

Compound 3A (retention time was 1.794 min, ee=100%): ¹H NMR (400 MHz, DMSO-d₆) δ 7.74-7.68 (m, 1H), 7.61 (s, 1H), 7.36-7.19 (m, 2H), 7.12-7.03 (m, 1H), 6.89-6.74(m, 1H), 5.40-5.32 (m, 1H), 4.47-4.38(m, 1H), 3.30 (s, 3H), 2.70-2.65 (m, 1H), 2.39 (s, 3H), 2.37-2.23 (m, 2H), 2.22 (s, 3H), 2.20-2.04 (m, 3H), 1.85-1.73 (m, 2H), 1.65-1.57 (m, 2H), 1.58-1.17 (m, 3H), 1.17-1.10 (m, 1H), 1.05-0.93 (m, 1H); LCMS: MS (ESI) m/z (M+H)+: 547.3.

Compound 3B (retention time was 1.924 min, ee=100%): ¹H NMR (400 MHz, DMSO-d₆) δ 7.74-7.65 (m, 1H), 7.61 (s, 1H), 7.36-7.19 (m, 2H), 7.12-7.00 (m, 1H), 6.89-6.75 (m, 1H), 5.40-5.30 (m, 1H), 4.47-4.31 (m, 1H), 3.30 (s, 3H), 2.70-2.65 (m, 1H), 2.39 (s, 3H), 2.37-2.23 (m, 2H), 2.22 (s, 3H), 2.20-2.04 (m, 3H), 1.85-1.73 (m, 2H), 1.65-1.52 (m, 2H), 1.58-1.17 (m, 3H), 1.17-1.10 (m, 1H), 1.05-0.94 (m, 1H); LCMS: MS (ESI) m/z (M+H)+: 547.4.

Compound 3C (retention time was 2.454 min, ee=99.68%): ¹H NMR (400 MHz, DMSO-d₆) δ 7.89-7.47 (m, 2H), 7.28 (br s, 2H), 7.12-7.01 (m, 1H), 6.90 (br s, 1H), 5.40 (br s, 1H), 4.47 (br s, 1H), 3.29-3.24 (m, 3H), 2.68 (br s, 1H), 2.39-2.31 (m, 5H), 2.27-2.04 (m, 7H), 1.90-1.56 (m, 4H), 1.43-1.40 (m, 2H), 1.20-0.96 (m, 2H); LCMS: MS (ESI) m/z (M+H)+: 547.3.

Compound 3D (retention time was 3.234 min, ee=99.37%): ¹H NMR (400 MHz, DMSO-d₆) δ 7.81-7.73 (m, 1H), 7.69 (s, 1H), 7.49-7.41 (m, 1H), 7.40-7.30 (m, 1H), 7.16-7.06 (m, 2H), 5.77-5.50 (m, 1H), 4.48-4.39 (m, 1H), 3.30 (s, 3H), 2.89-2.81 (m, 1H), 2.42 (s, 3H), 2.39-2.27 (m, 2H), 2.25 (s, 3H), 2.23-2.11 (m, 3H), 2.07-2.18 (m, 1H), 1.95-1.91 (m, 1H), 1.88-1.79 (m, 1H), 1.73 (br s, 1H), 1.49-1.30 (m, 4H), 0.91-0.86 (m, 1H)); LCMS: MS (ESI) m/z (M+H)+: 547.4.

Analysis method: Chiralpak AS-3 100×4.6 mm I.D., 3 μm; mobile phase: A: supercritical carbon dioxide, B: 0.05% diethylamine in ethanol; gradient: from 5% B to 40% B in 4 minutes, 40% B for 2.5 min, from 5% B back to 1.5% B in 1.5 min; flow rate: 2.8 mL/min; column temperature: 35° C.; wavelength: 220 nm.

### EXAMPLE 4

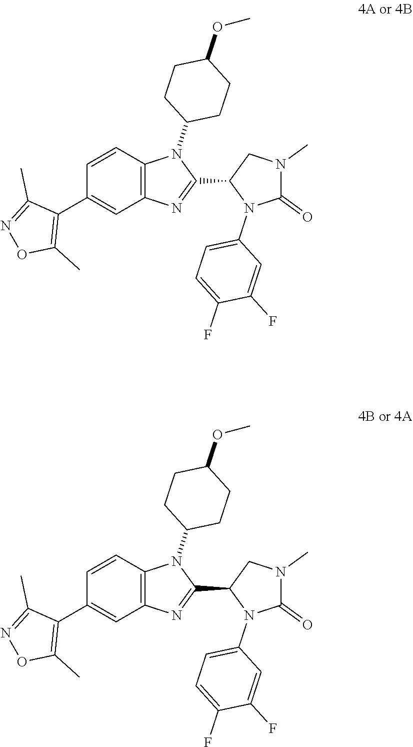

Route of Synthesis:

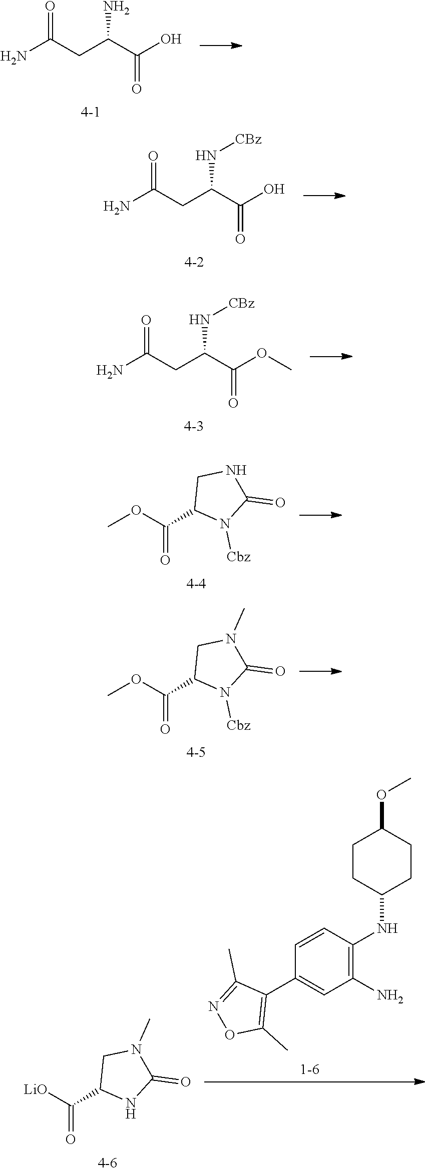

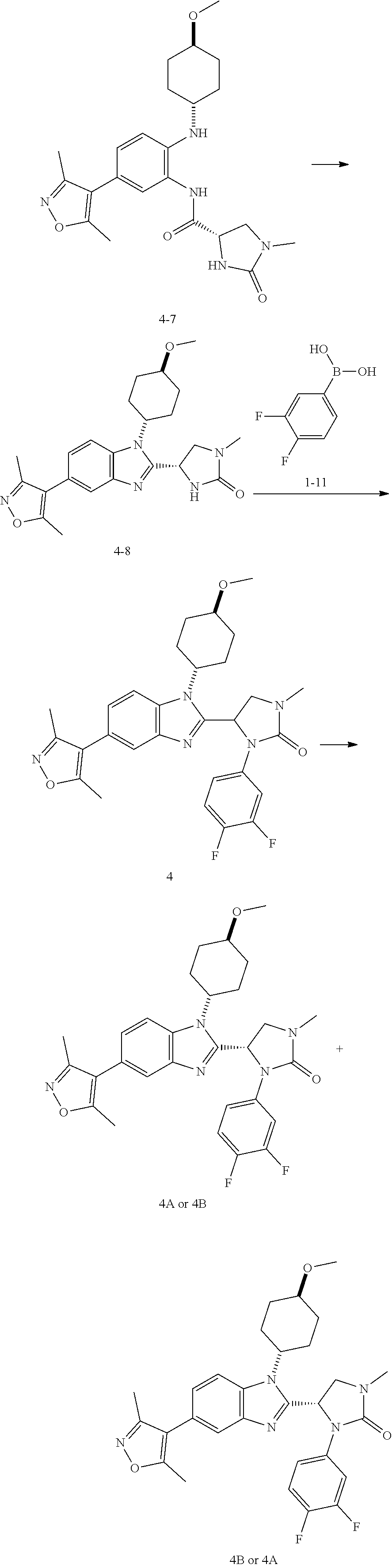

### Preparation of Compound 4-2

To a solution of compound 4-1 (10.0 g, 75.69 mmol, 1 eq), Na₂CO₃ (16.04 g, 151.38 mmol, 2 eq), 1,4-dioxane (60 mL) and water (60 mL) was added CbzCl (16.20 g, 94.96 mmol, 13.5 mL, 1.25 eq) at 0° C., and the resulting reaction solution was warmed to 20° C. and stirred for 16 hours. The reaction solution was poured into water (300 mL), and petroleum ether (200 mL) was added, and the layers were separated. To the aqueous phase was added 2N HCl, and a white solid was precipitated. The mixture was filtered to give compound 4-2. LCMS: MS (ESI) m/z (M+H)+: 266.9.

### Preparation of Compound 4-3

To a solution of compound 4-2 (1.5 g, 5.63 mmol, 1 eq), dichloromethane (30 mL) and methanol (3 mL) was added (trimethylsilyl)diazomethane (2 M, 4.20 mL, 1.49 eq) at 20° C., and the resulting reaction solution was stirred at 20° C. for 16 hours. The reaction solution was concentrated under reduced pressure to give compound 4-3. LCMS: MS (ESI) m/z (M+H)+: 281.1.

### Preparation of Compound 4-4

To a solution of DBU (2.93 g, 19.24 mmol, 2.9 mL, 2.57 eq) in tetrahydrofuran (160 mL) was added compound 4-3 (2.1 g, 7.49 mmol, 1 eq) at 0° C., and then (diacetoxyiodo)benzene (4.83 g, 15.00 mmol, 2 eq) was added, and the resulting reaction solution was stirred at 0° C. for 15 minutes. Water (1.00 g, 55.51 mmol, 1 mL, 7.41 eq) was added, and the mixture was stirred for 30 minutes. The reaction solution was concentrated under reduced pressure, and to the residue was added ethyl acetate (50 mL). The mixture was washed with water (50 mL) and saturated brine (50 mL), dried over anhydrous sodium sulfate, and filtered. The filtrate was concentrated under reduced pressure. To the residue was added n-hexane/methyl tert-butyl ether/ethyl acetate (5 mL, v:v:v=2:2:1). The mixture was stirred at room temperature for 15 minutes, and filtered. The filter cake was collected to give compound 4-4. LCMS: MS (ESI) m/z (M+H)+: 279.1.

### Preparation of Compound 4-5

To a solution of compound 4-4 (1.4 g, 5.03 mmol, 1 eq) in acetonitrile (20 mL) were added potassium carbonate (1.40 g, 10.13 mmol, 2.01 eq) and iodomethane (4.56 g, 32.13 mmol, 2.0 mL, 6.39 eq) at 30° C., and the resulting reaction solution was stirred at 30° C. for 16 hours. To the reaction solution was added additional iodomethane (2.28 g, 16.06 mmol, 1.0 mL, 3.19 eq), and the resulting reaction solution was stirred at 30° C. for 16 hours. The reaction solution was filtered, and ethyl acetate (100 mL) was added. The mixture was washed with water (50 mL) and saturated brine (50 mL), dried over anhydrous sodium sulfate, and filtered. The filtrate was concentrated to dryness under reduced pressure to give compound 4-5. LCMS: MS (ESI) m/z (M+H)+: 293.1.

### Preparation of Compound 4-6

To a solution of compound 4-5 (0.6 g, 2.05 mmol, 1 eq) in tetrahydrofuran (3 mL) was added a solution of lithium hydroxide monohydrate (180 mg, 4.29 mmol, 2.09 eq) in water (3 mL), and the resulting reaction solution was stirred at 30° C. for 16 hours. To the reaction solution was added water (20 mL), and the mixture was directly vacuum-lyophilized to give compound 4-6. LCMS: MS (ESI) m/z (M+H)+: 145.1.

### Preparation of Compound 4-7

To a solution of compound 4-6 (400 mg, 2.67 mmol, 4.20 eq) in N,N-dimethylformamide (6 mL) were added HATU (1.00 g, 2.63 mmol, 4.15 eq), compound 1-6 (200 mg, 634.10 μmol, 1 eq) and triethylamine (290.80 mg, 2.87 mmol, 0.4 mL, 4.53 eq), and the resulting reaction solution was stirred at 30° C. for 16 hours. To the reaction solution were added ethyl acetate (30 mL) and water (30 mL), and the layers were separated. The organic phase was washed with saturated brine (20 mL), dried over anhydrous sodium sulfate, and filtered. The filtrate was concentrated under reduced pressure. The residue was purified by column chromatography (dichloromethane/tetrahydrofuran=1/0˜0/1) to give compound 4-7. LCMS: MS (ESI) m/z (M+H)+: 442.2.

### Preparation of Compound 4-8

A solution of compound 4-7 (270 mg, 611.52 μmol, 1 eq) and acetic acid (3 mL) was heated to 80° C. and stirred for 16 hours. The reaction solution was concentrated under reduced pressure to give compound 4-8. LCMS: MS (ESI) m/z (M+H)+: 424.1.

### Preparation of Compounds 4A and 4B

To a solution of compound 4-8 (100 mg, 236.12 μmol, 1 eq), dichloromethane (2 mL) and acetonitrile (2 mL) was added DBU (80 mg, 525.50 μmol, 79.21 μL, 2.23 eq) at 30° C., and the mixture was stirred for 15 minutes. CU-TMEDA CATALYST (II) (22 mg, 47.37 μmol, 2.01e-1 eq) was added, and the mixture was stirred for 15 minutes. Compound 1-11 (75 mg, 474.95 μmol, 2.01 eq) was added, and the resulting reaction solution was stirred at 30° C. for 16 hours. The reaction solution was filtered, and the filtrate was concentrated under reduced pressure. The residue was purified by column chromatography (dichloromethane/tetrahydrofuran=1/0˜0/1) to give a product. The product was analyzed to be racemic compound 4 (racemization was presumed to have occurred during the preparation of 4-7 or 4-8 or compound 4) by detection with supercritical fluid chromatography (Chiralpak OD-3 100×4.6 mm I.D., 3 μm; mobile phase: A: supercritical carbon dioxide, B: 0.05% diethylamine in ethanol; gradient: from 5% B to 40% B in 4 minutes, 40% B for 2.5 min, from 5% B back to 1.5% B in 1.5 min; flow rate: 2.8 mL/min; column temperature: 35° C.; wavelength: 220 nm). Compound 4 was isolated by SFC (column: DAICEL CHIRALCEL OD-H (250 mm*30 mm, 5 μm); mobile phase: [0.1% NH₃H₂O EtOH]; 40%-40%) to give chiral isomers compound 4A and compound 4B.

Compound 4A (retention time was 3.367 min, ee=98.46%): ¹H NMR (400 MHz, CDCl₃) δ 7.68-7.56 (m, 2H), 7.52 (d, J=8.5 Hz, 1H), 7.20-7.09 (m, 2H), 7.01-6.92 (m, 1H), 5.78-5.70 (m, 1H), 4.46-4.34 (m, 1H), 4.06 (t, J=10.0 Hz, 1H), 3.56 (dd, J=7.2, 9.4 Hz, 1H), 3.41 (s, 3H), 3.35-3.25 (m, 1H), 3.04 (s, 3H), 2.43 (s, 3H), 2.41-2.33 (m, 1H), 2.29 (s, 3H), 2.27-2.10 (m, 2H), 1.91-1.86 (m, 1H), 1.53-1.41 (m, 2H), 1.41-1.31 (m, 2H); LCMS: MS (ESI) m/z (M+H)+: 536.3.

Compound 4B (retention time was 3.735 min, ee=84.6%): ¹H NMR (400 MHz, CDCl₃) δ 7.66-7.56 (m, 2H), 7.52 (d, J=8.5 Hz, 1H), 7.20-7.09 (m, 2H), 7.06-6.96 (m, 1H), 5.78-5.68(m, 1H), 4.47-4.37 (m, 1H), 4.06 (t, J=10.0 Hz, 1H), 3.56 (dd, J=7.2, 9.4 Hz, 1H), 3.41 (s, 3H), 3.35-3.26 (m, 1H), 3.04 (s, 3H), 2.43 (s, 3H), 2.41-2.33 (m, 1H), 2.29 (s, 3H), 2.27-2.11 (m, 2H), 1.91-1.87 (m, 1H), 1.44-1.35 (m, 4H); LCMS: MS (ESI) m/z (M+H)+: 536.4.

Analysis method: Chiralpak OD-3 100×4.6 mm I.D., 3 μm; mobile phase: A: supercritical carbon dioxide, B: 0.05% diethylamine in ethanol; gradient: from 5% B to 40% B in 4 minutes, 40% B for 2.5 min, from 5% B back to 1.5% B in 1.5 min; flow rate: 2.8 mL/min; column temperature: 35° C.; wavelength: 220 nm.

### EXAMPLE 6

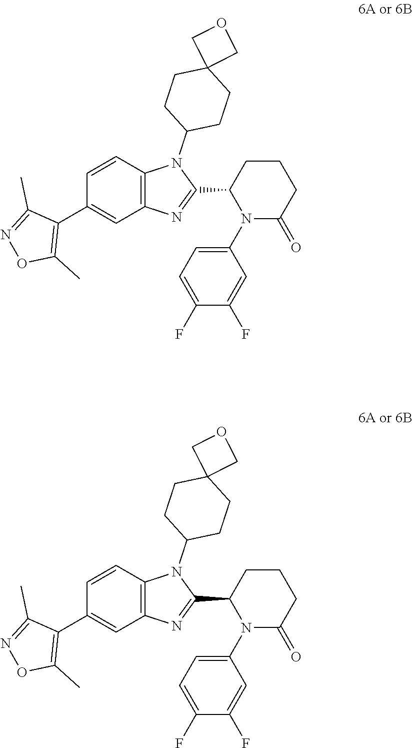 [SMILES: Cc1noc(C)c1-c1ccc2c(c1)nc([C@@H]1CCCC(=O)N1c1ccc(F)c(F)c1)n2C1CCC2(CC1)COC2 ; Cc1noc(C)c1-c1ccc2c(c1)nc([C@H]1CCCC(=O)N1c1ccc(F)c(F)c1)n2C1CCC2(CC1)COC2]

Route of Synthesis:

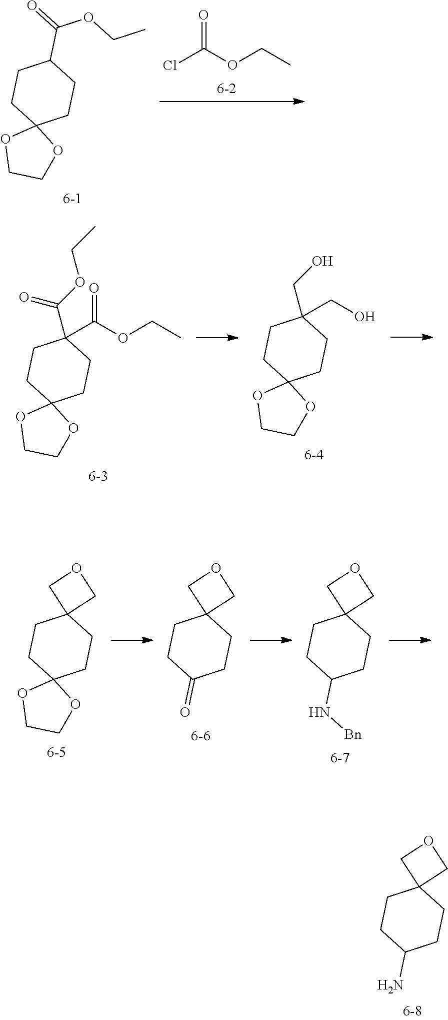

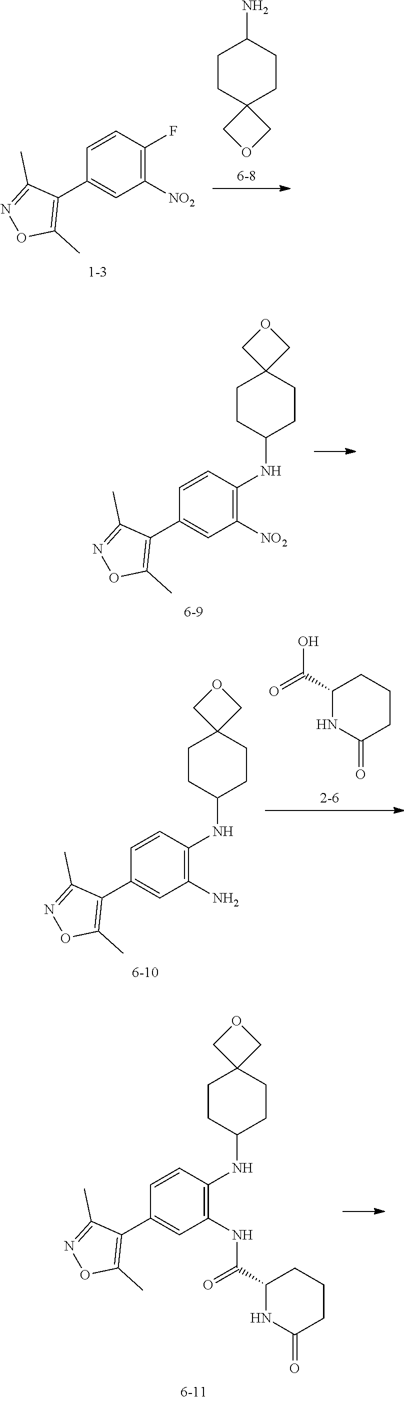

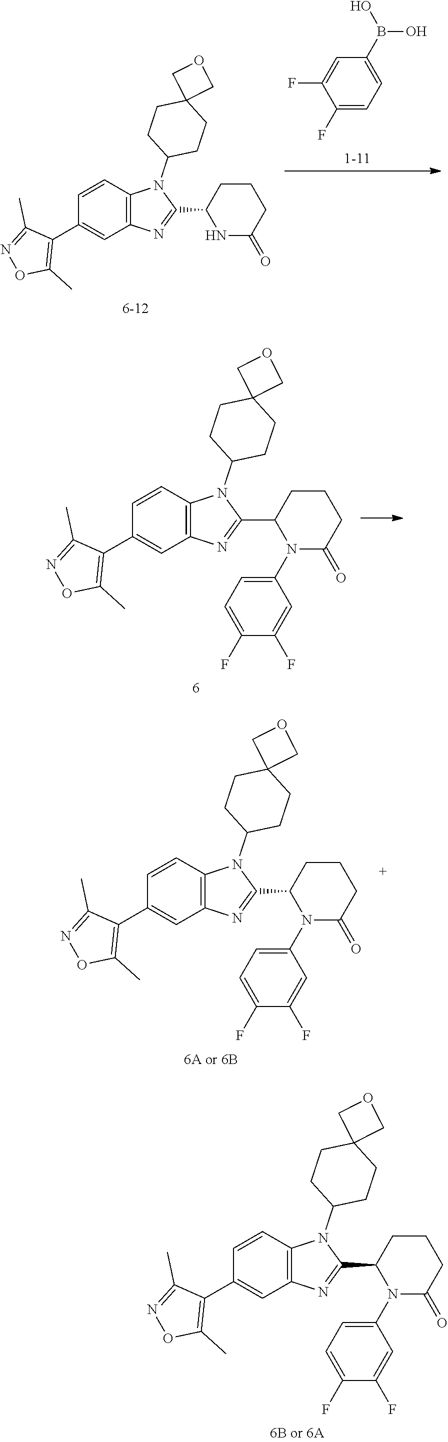 [SMILES: Cc1noc(C)c1-c1ccc2c(c1)nc([C@@H]1CCCC(=O)N1)n2C1CCC2(CC1)COC2 ; OB(O)c1ccc(F)c(F)c1 ; Cc1noc(C)c1-c1ccc2c(c1)nc(C1CCCC(=O)N1c1ccc(F)c(F)c1)n2C1CCC2(CC1)COC2 ; Cc1noc(C)c1-c1ccc2c(c1)nc([C@@H]1CCCC(=O)N1c1ccc(F)c(F)c1)n2C1CCC2(CC1)COC2 ; Cc1noc(C)c1-c1ccc2c(c1)nc([C@H]1CCCC(=O)N1c1ccc(F)c(F)c1)n2C1CCC2(CC1)COC2]

### Preparation of Compound 6-3

Compound 6-1 (24 g, 112.01 mmol, 1 eq) was dissolved in anhydrous tetrahydrofuran (750 mL), and the mixture was cooled to −78° C. Lithium diisopropylamide (2 M, 73 mL, 1.3 eq) was added into the above reaction solution. The reaction solution was stirred at −78° C. for 1 hour, warmed to 0° C. (the bottle was raised to the liquid level) and stirred for 10 minutes, and cooled to −78° C. again. Compound 6-2 (20 g, 184.29 mmol, 17.54 mL, 1.65 eq) was added to the above reaction solution. The mixture was stirred at −78° C. for 1 hour, and then gradually warmed to 25° C. and stirred for another 16 hours. The reaction was quenched with saturated ammonium chloride solution (100 mL), and water (200 mL) was added. The layers were separated. The aqueous phase was extracted with ethyl acetate (200 mL*3). The above-mentioned organic phases were combined, washed with saturated brine (200 mL), dried over anhydrous sodium sulfate, and filtered. The filtrate was rotary evaporated to dryness, and the residue was purified by column chromatography (petroleum ether/ethyl acetate=1:0-9:1) to give compound 6-3. LCMS: MS (ESI) m/z (M+H)+: 287.2.

### Preparation of Compound 6-4

Compound 6-3 (34 g, 118.75 mmol, 1 eq) was added to a suspension of lithium aluminum hydride (5.44 g, 143.33 mmol, 1.21 eq) in anhydrous tetrahydrofuran (500 mL) at 0° C. After the reaction solution was stirred at 0° C. for 1 hour, LAH (5.44 g, 143.33 mmol, 1.21 eq) was added, and the reaction solution was stirred at 25° C. for 1 hour. To the reaction solution was added pasty sodium sulfate in portions, and the mixture was filtered. The filtrate was rotary evaporated to dryness, and ethyl acetate (200 mL) and water (100 mL) were added. After the layers were separated, the aqueous phase was extracted with ethyl acetate (100 mL*3). The above-mentioned organic phases were combined, dried over anhydrous sodium sulfate, and filtered. The filtrate was rotary evaporated to dryness to give compound 6-4. LCMS: MS (ESI) m/z (M+H)+: 203.2.

### Preparation of Compound 6-5

n-BuLi (2.5 M, 16.00 mL, 1.01 eq) was added to a solution of compound 6-4 (8 g, 39.56 mmol, 1 eq) in anhydrous tetrahydrofuran (80 mL) at 0° C., and the reaction solution was stirred at 0° C. for 30 minutes. A solution of p-toluenesulfonyl chloride (7.55 g, 39.60 mmol, 1 eq) in anhydrous tetrahydrofuran (15 mL) was added dropwise to the above reaction solution. The reaction solution was gradually warmed to 25° C., stirred for 1 hour, and cooled to 0° C. again. n-BuLi (2.5 M, 23.73 mL, 1.5 eq) was added dropwise to the above reaction solution. The reaction solution was heated to 70° C. (reflux) and stirred for 16 hours. After the reaction solution was cooled to room temperature, the reaction was quenched with saturated sodium bicarbonate solution (˜5 mL), and water (50 mL) and ethyl acetate (50 mL) were added. The layers were separated, and then the aqueous phase was extracted with ethyl acetate (50 mL*3). The above-mentioned organic phases were combined, washed with saturated brine (50 mL), dried over anhydrous sodium sulfate, and filtered. The filtrate was rotary evaporated to dryness, and the residue was purified by column chromatography (petroleum ether/ethyl acetate=1:0˜0:1) to give compound 6-5. LCMS: MS (ESI) m/z (M+H)+: 185.1.

### Preparation of Compound 6-6

HCl (29.58 g, 1.62 mmol, 29 mL, 0.2% mass fraction, 1.49e-1 eq) was added to a solution of compound 6-5 (2 g, 10.86 mmol, 1 eq) in anhydrous tetrahydrofuran (15 mL), and the mixture was stirred at 25° C. for 16 hours. The reaction solution was adjusted to a pH of 7 with saturated sodium bicarbonate solution, and ethyl acetate (20 mL) was added. The layers were separated, and the aqueous phase was extracted with ethyl acetate (10 mL*3). The above-mentioned organic phases were combined, washed with saturated brine (20 mL), dried over anhydrous sodium sulfate, and filtered. The filtrate was rotary evaporated to dryness, and the residue was purified by column chromatography (petroleum ether/ethyl acetate=1:0˜1:1) to give compound 6-6. ¹HNMR (400 MHz, CDCl₃) δ ppm 4.56 (s, 4 H), 2.31-2.37 (m, 4 H), 2.15-2.21 (m, 4 H).

### Preparation of Compound 6-7

Benzylamine (1.60 g, 14.93 mmol, 1.63 mL, 1.31 eq) and sodium acetate borohydride (2.88 g, 13.59 mmol, 1.19 eq) were added to a solution of compound 6-6 (1.6 g, 11.41 mmol, 1 eq) in 1,2-dichloroethane (30 mL), and the reaction solution was stirred at 25° C. for 1 hour. To the reaction solution was added saturated sodium bicarbonate solution (10 mL) to quench the reaction. The mixture was filtered, and the layers were separated. The aqueous phase was extracted with dichloromethane (30 mL*3). The above-mentioned organic phases were combined, dried over anhydrous sodium sulfate, and filtered. The filtrate was rotary evaporated to dryness, and the residue was purified by column chromatography (dichloromethane/methanol=1:0˜10:1) to give compound 6-7. LCMS: MS (ESI) m/z (M+H)+: 232.2.

### Preparation of Compound 6-8

Wet Pd/C (1.4 g, 10% purity) was added to a solution of compound 6-7 (0.7 g, 3.03 mmol, 1 eq) in anhydrous methanol (20 mL), and the mixture was stirred under a hydrogen atmosphere (balloon) at 20° C. for 16 hours. The reaction solution was directly filtered, and the filtrate was rotary evaporated to dryness to give compound 6-8. LCMS: MS (ESI) m/z (M+H)+: 142.1.

### Preparation of Compound 6-9

Cesium carbonate (5.32 g, 16.33 mmol, 3.03 eq) was added to a solution of compound 1-3 (1.90 g, 8.04 mmol, 1.49 eq) and compound 6-8 (760 mg, 5.38 mmol, 1 eq) in anhydrous tetrahydrofuran (50 mL), and the mixture was stirred at 70° C. for 16 hours. The reaction solution was cooled to room temperature and then poured into water (50 mL). The layers were separated, and the aqueous phase was extracted with ethyl acetate (20 mL*3). The above-mentioned organic phases were combined, washed with saturated brine (20 mL), dried over anhydrous sodium sulfate, and filtered. The filtrate was rotary evaporated to dryness, and the residue was purified by column chromatography (petroleum ether/dichloromethane=1:0˜0:1) to give compound 6-9. LCMS: MS (ESI) m/z (M+H)+: 358.2.

### Preparation of Compound 6-10

Compound 6-9 (1.3 g, 3.64 mmol, 1 eq) was added to a solution of aqueous ammonia (9.10 g, 72.70 mmol, 10 mL, 28% purity, 19.99 eq) and sodium hydrosulfite (6.4 g, 36.76 mmol, 8.00 mL, 10.11 eq) in tetrahydrofuran (10 mL) and water (10 mL), and the mixture was stirred at 20° C. for 2 hours. The reaction solution was poured into water (10 mL), and the mixture was extracted with ethyl acetate (10 mL*3). The organic phases were combined, washed with saturated brine (20 mL), dried over anhydrous sodium sulfate, and filtered. The filtrate was rotary evaporated to dryness to give compound 6-10. LCMS: MS (ESI) m/z (M+H)+: 328.2.

### Preparation of Compound 6-11

Compound 2-6 (870.00 mg, 6.08 mmol, 1.99 eq), HATU (2.32 g, 6.10 mmol, 2 eq) and triethylamine (1.45 g, 14.37 mmol, 2 mL, 4.70 eq) were added to a solution of compound 6-10 (1 g, 3.05 mmol, 1 eq) in anhydrous DMF (50 mL), and the mixture was stirred at 20° C. for 2 hours. The reaction solution was concentrated under reduced pressure to give a crude product. To the above-mentioned crude product were added water (50 mL) and dichloromethane (100 mL), and HCl (1 M) was added to adjust to a pH of 4. The layers were separated, and the organic phase was adjusted to a pH of 7 with saturated aqueous sodium bicarbonate solution. After the layers were separated, the organic phase was washed with saturated brine (50 mL), dried over anhydrous sodium sulfate, and filtered. The filtrate was rotary evaporated to dryness, and the residue was purified by column chromatography (dichloromethane/methanol=1:0˜10:1) to give compound 6-11. LCMS: MS (ESI) m/z (M+H)+: 453.4.

### Preparation of Compound 6-12

Compound 6-11 (1.33 g, 2.94 mmol, 1 eq) was dissolved in AcOH (20 mL), and the mixture was heated to 80° C. and stirred for 16 hours. The reaction solution was rotary evaporated to dryness, and the residue was purified by column chromatography (petroleum ether/ethyl acetate=1:0˜0:1) to give compound 6-12. LCMS: MS (ESI) m/z (M+H)+: 435.3.

### Preparation of Compounds 6A and 6B

DBU (606.00 mg, 3.98 mmol, 600 ∞L, 3.14 eq) was added to a solution of compound 6-12 (550 mg, 1.27 mmol, 1 eq) in dichloromethane (5 mL) and acetonitrile (10 mL). After the reaction solution was stirred at 20° C. for 15 minutes, CU-TMEDA CATALYST (II) (800 mg, 1.72 mmol, 1.36 eq) was added. The reaction solution was stirred at 20° C. for another 15 minutes, and then compound 1-11 (1 g, 6.33 mmol, 5 eq) was added to the above reaction solution. The mixture was stirred at 20° C. for another 20 hours. The reaction solution was filtered, and the filtrate was concentrated under reduced pressure. The residue was purified by column chromatography (dichloromethane/tetrahydrofuran=1:0-1:1) to give a product. The product was analyzed to be racemic compound 6 (racemization was presumed to have occurred during the preparation of 6-11 or 6-12 or compound 6) by detection with supercritical fluid chromatography (Chiralpak AS-3 100×4.6 mm I.D., 3 μm; mobile phase: A: supercritical carbon dioxide, B: 0.05% diethylamine in ethanol; gradient: from 5% B to 40% B in 4 minutes, 40% B for 2.5 min, from 5% B back to 1.5% B in 1.5 min; flow rate: 2.8 mL/min; column temperature: 35° C.; wavelength: 220 nm). Compound 6 was isolated by SFC (column: DAICEL CHIRALPAK AS (250 mm*30 mm, 10 μm); mobile phase: [0.1% NH₃H₂O EtOH]; 35%-35%) to give chiral isomers compound 6A and compound 6B.

Compound 6A (retention time was 2.804 min, ee=100%): ¹H NMR (400 MHz, DMSO-d₆) δ ppm 7.69 (s, 1 H), 7.50-7.48 (m, 1 H), 7.40-7.29 (m, 2 H) , 7.18-7.13 (m, 1 H), 7.04-7.02 (m, 1 H), 5.73-5.71 (m, 1 H), 4.56-4.47 (m, 2 H) , 4.35-4.28(m, 1 H), 4.27 (s, 2 H), 2.56-2.49 (m, 1 H), 2.40 (s, 3 H), 2.33-2.31 (m, 1 H), 2.23 (s, 3 H), 2.21-2.18 (m, 1 H), 2.13-2.11 (m, 2 H), 2.06-1.92(m, 4 H), 1.77-1.74 (m, 2 H), 1.71-1.57 (m, 2 H), 1.13-1.10 (m, 1 H); LCMS: MS (ESI) m/z (M+H)+: 547.4.

Compound 6B (retention time was 4.150 min, ee=100%): ¹H NMR (400 MHz, DMSO-d₆) δ ppm 7.69 (s, 1 H), 7.50-7.47 (m, 1 H), 7.41-7.29 (m, 2 H), 7.15-7.11 (m, 1 H), 7.04-7.00 (m, 1 H), 5.73-5.72 (m, 1 H), 4.55-4.48 (m, 2 H), 4.35-4.28 (m, 1 H), 4.27 (s, 2 H), 2.56-2.48 (m, 1 H), 2.40 (s, 3 H), 2.34-2.30 (m, 1 H), 2.23 (s, 3 H), 2.22-2.16 (m, 1H), 2.13-2.10(m, 2H), 2.08-1.93(m, 4H), 1.77-1.75 (m, 2H), 1.70-1.59 (m, 2H), 1.13-1.11 (m, 1H); LCMS: MS (ESI) m/z (M+H)+: 547.4.

Analysis method: Chiralpak AS-3 100×4.6 mm I.D., 3 μm; mobile phase: A: supercritical carbon dioxide, B: 0.05% diethylamine in ethanol; gradient: from 5% B to 40% B in 4 minutes, 40% B for 2.5 min, from 5% B back to 1.5% B in 1.5 min; flow rate: 2.8 mL/min; column temperature: 35° C.; wavelength: 220 nm.

### EXAMPLE 7

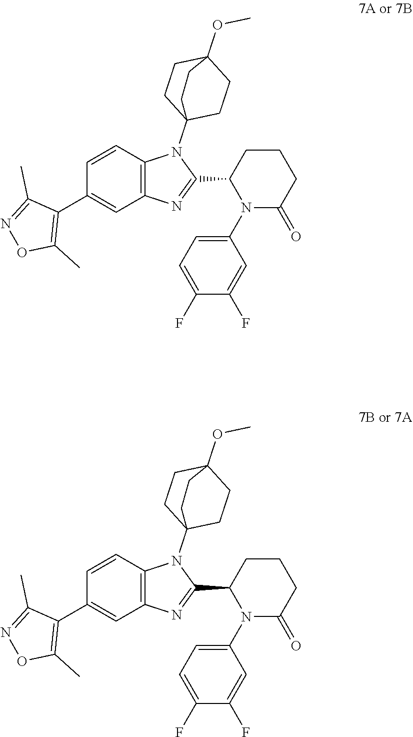 [SMILES: COC12CCC(n3c([C@@H]4CCCC(=O)N4c4ccc(F)c(F)c4)nc4cc(-c5c(C)noc5C)ccc43)(CC1)CC2 ; COC12CCC(n3c([C@H]4CCCC(=O)N4c4ccc(F)c(F)c4)nc4cc(-c5c(C)noc5C)ccc43)(CC1)CC2]

Route of Synthesis:

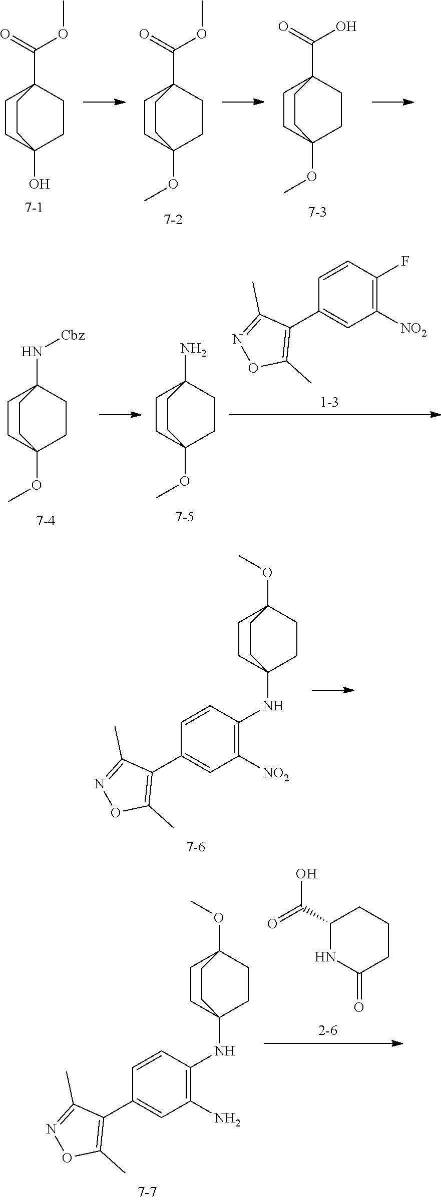

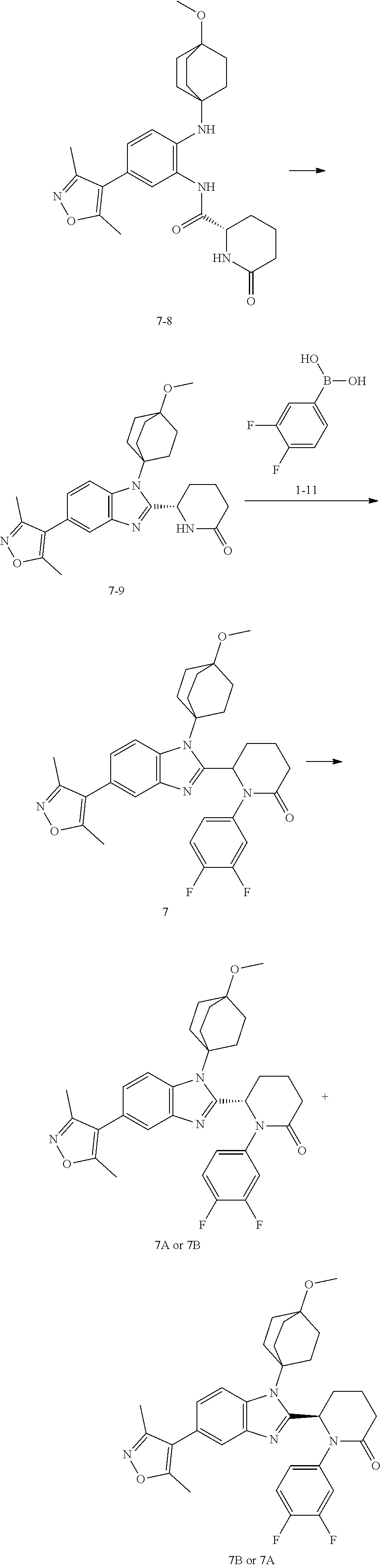 [SMILES: COC12CCC(Nc3ccc(-c4c(C)noc4C)cc3NC(=O)[C@@H]3CCCC(=O)N3)(CC1)CC2 ; COC12CCC(n3c([C@@H]4CCCC(=O)N4)nc4cc(-c5c(C)noc5C)ccc43)(CC1)CC2 ; OB(O)c1ccc(F)c(F)c1 ; COC12CCC(n3c(C4CCCC(=O)N4c4ccc(F)c(F)c4)nc4cc(-c5c(C)noc5C)ccc43)(CC1)CC2 ; COC12CCC(n3c([C@@H]4CCCC(=O)N4c4ccc(F)c(F)c4)nc4cc(-c5c(C)noc5C)ccc43)(CC1)CC2 ; COC12CCC(n3c([C@H]4CCCC(=O)N4c4ccc(F)c(F)c4)nc4cc(-c5c(C)noc5C)ccc43)(CC1)CC2]

### Preparation of Compound 7-2

To a solution of compound 7-1 (940 mg, 5.10 mmol, 1 eq) in N,N-dimethylformamide (15 mL) were added sodium hydride (300 mg, 7.50 mmol, 60% purity, 1.47 eq) and iodomethane (5.70 g, 40.16 mmol, 2.50 mL, 7.87 eq), and the resulting reaction solution was stirred at 15° C. for 16 hours. The reaction solution was poured into water (60 mL), and the mixture was extracted with ethyl acetate (60 mL*3). The organic phase were washed with saturated brine (50 mL), dried over anhydrous sodium sulfate, and filtered. The filtrate was concentrated under reduced pressure to give compound 7-2. ¹H NMR (400 MHz, CDCl₃) δ 3.57 (s, 3H), 3.11 (s, 3H), 1.89-1.79 (m, 6H), 1.65-1.56 (m, 6H).

### Preparation of Compound 7-3

To a solution of compound 7-2 (950 mg, 4.79 mmol, 1 eq) in tetrahydrofuran (10 mL) was added a solution of lithium hydroxide monohydrate (410 mg, 9.77 mmol, 2.04 eq) in water (10 mL), and the resulting reaction solution was stirred at 15° C. for 16 hours. To the reaction solution were added ethyl acetate (10 mL) and water (10 mL) for extraction, and the layers were separated. To the aqueous phase was added concentrated hydrochloric acid (pH of about 3) dropwise, and the mixture was extracted with ethyl acetate (15 mL*3). The organic phases were combined, washed with saturated brine (30 mL), dried over anhydrous sodium sulfate, and filtered. The filtrate was concentrated under reduced pressure to give compound 7-3. ¹H NMR (400 MHz, CDCl₃) δ 3.19 (s, 3H), 1.99-1.90 (m, 6H), 1.75-1.64 (m, 6H).

### Preparation of Compound 7-4

To a solution of compound 7-3 (800 mg, 4.34 mmol, 1 eq) in 1,4-dioxane (10 mL) were added N,N-diisopropylethylamine (1.14 g, 8.84 mmol, 1.54 mL, 2.04 eq), diphenylphosphoryl azide (1.79 g, 6.51 mmol, 1.41 mL, 1.5 eq) and benzyl alcohol (2.35 g, 21.71 mmol, 2.26 mL, 5 eq). The resulting reaction solution was heated to 80° C. and stirred for 16 hours. The reaction solution was concentrated under reduced pressure. The residue was purified by column chromatography (petroleum ether/ethyl acetate=1/0˜3/1) to give compound 7-4. ¹H NMR (400 MHz, CDCl₃) δ 7.34-7.28 (m, 5H), 5.03 (br s, 2H), 4.57 (br s, 1H), 3.18 (s, 3H), 2.02-1.92 (m, 6H), 1.80-1.74 (m, 6H); LCMS: MS (ESI) m/z (M+H)+: 290.1.

### Preparation of Compound 7-5

To a solution of compound 7-4 (1.5 g, 5.18 mmol, 1 eq) in methanol (20 mL) was added palladium-carbon (150 mg, 10% purity). The resulting reaction solution was purged with hydrogen three times, and stirred under a hydrogen atmosphere (balloon) at 15° C. for 16 hours. The reaction solution was filtered, and the filtrate was concentrated under reduced pressure to give compound 7-5. ¹H NMR (400 MHz, CDCl₃) δ 3.18 (s, 3H), 1.79-1.60 (m, 12H); LCMS: MS (ESI) m/z (M+H)+: 156.2.

### Preparation of Compound 7-6

To a solution of compound 1-3 (300 mg, 1.27 mmol, 1 eq) in tetrahydrofuran (12 mL) were added triethylamine (390 mg, 3.85 mmol, 536.45 μL, 3.03 eq) and compound 7-5 (900 mg, 5.80 mmol, 4.56 eq), and the resulting reaction solution was heated to 70° C. and stirred for 16 hours. The reaction solution was concentrated under reduced pressure. The residue was purified by column chromatography (petroleum ether/ethyl acetate=1/0˜1/1) to give compound 7-6. LCMS: MS (ESI) m/z (M+H)+: 372.3.

### Preparation of Compound 7-7

To a solution of sodium hydrosulfite (938 mg, 5.39 mmol, 1.17 mL, 10.01 eq), aqueous ammonia (10.91 mmol, 1.5 mL, 20.25 eq), tetrahydrofuran (5 mL) and water (5 mL) was added compound 7-6 (200 mg, 538.46 μmol, 1 eq), and the resulting reaction solution was stirred at 15° C. for 1 hours. The reaction solution was poured into water (15 mL), and the mixture was extracted with ethyl acetate (20 mL*2). The organic phase was washed with saturated brine (15 mL), dried over anhydrous sodium sulfate, and filtered. The filtrate was concentrated under reduced pressure to give compound 7-7. LCMS: MS (ESI) m/z (M+H)+: 342.3.

### Preparation of Compound 7-8

To a solution of compound 2-6 (120 mg, 838.34 μmol, 1.91 eq), acetonitrile (5 mL) and N,N-dimethylformamide (1 mL) were added 1-methylimidazole (180 mg, 2.19 mmol, 174.76 μL, 4.99 eq), compound 7-7 (150 mg, 439.31 μmol, 1 eq) and HATU (275 mg, 980.12 μmol, 2.23 eq), and the resulting reaction solution was stirred at 15° C. for 18 hours. The reaction solution was poured into water (30 mL). The mixture was extracted with ethyl acetate (30 mL*2), and the layers were separated. The organic phase was washed sequentially with saturated sodium bicarbonate solution (30 mL) and saturated brine (30 mL), dried over anhydrous sodium sulfate, and filtered. The filtrate was concentrated under reduced pressure. The residue was purified by column chromatography (dichloromethane/tetrahydrofuran=1/0˜0/1) to give compound 7-8. LCMS: MS (ESI) m/z (M+H)+: 467.4.

### Preparation of Compound 7-9

To a microwave tube were added compound 7-8 (270 mg, 578.69 μmol, 1 eq) and acetic acid (3 mL), and the resulting reaction solution was heated to 150° C. and reacted with microwave for 0.5 hours. The reaction solution was concentrated under reduced pressure. To the residue was added dichloromethane (50 mL). The mixture was washed with saturated sodium bicarbonate solution (50 mL), dried over anhydrous sodium sulfate, and filtered. The filtrate was concentrated under reduced pressure. The residue was purified by column chromatography (dichloromethane/tetrahydrofuran=5/10/1) to give compound 7-9. LCMS: MS (ESI) m/z (M+H)+: 449.3.

### Preparation of Compounds 7A and 7B

To a solution of compound 7-9 (100 mg, 222.94 μmol, 1 eq) and methanol (2 mL) was added DBU (75 mg, 492.65 μmol, 74.26 μL, 2.21 eq) at 15° C., and the mixture was stirred for 15 minutes. CU-TMEDA CATALYST (II) (21 mg, 45.22 μmol, 2.03e-1 eq) was added, and the mixture was stirred for 15 minutes. Compound 1-11 (150 mg, 949.91 μmol, 4.26 eq) was added, and the resulting reaction solution was stirred at 10° C. for 16 hours. The reaction solution was filtered, and the filtrate was concentrated under reduced pressure. The residue was purified by column chromatography (dichloromethane/tetrahydrofuran=5/1˜0/1) to give a product. The product was analyzed to be racemic compound 7 (racemization was presumed to have occurred during the preparation of 7-8 or 7-9 or compound 7) by detection with supercritical fluid chromatography (Chiralpak OD-3 100×4.6 mm I.D., 3 μm; mobile phase: A: supercritical carbon dioxide, B: 0.05% diethylamine in ethanol; gradient: from 5% B to 40% B in 4 minutes, 40% B for 2.5 min, from 5% B back to 1.5% B in 1.5 min; flow rate: 2.8 mL/min; column temperature: 35° C.; wavelength: 220 nm). Compound 7 was isolated by SFC (column: DAICEL CHIRALCEL OD-H (250 mm*30 mm, 5 um); mobile phase: [0.1% NH₃H₂O EtOH]; 35%-35%) to give chiral isomers compound 7A and compound 7B.

Compound 7A (retention time was 3.861 min, ee=99.54%): ¹H NMR (400 MHz, CDCl₃) δ 7.60 (s, 1H), 7.55 (d, J=8.8 Hz, 1H), 7.05-6.93 (m, 3H), 6.87-6.84 (m, 1H), 5.44 (br s, 1H), 3.15 (s, 3H), 2.78-2.71 (m, 1H), 2.58-2.45 (m, 1H), 2.39 (s, 3H), 2.35-2.07 (m, 12H), 1.84-1.81 (m, 6H), 1.73-1.67 (m, 1H); LCMS: MS (ESI) m/z (M+H)+: 561.4.

Compound 7B (retention time was 4.012 min, ee=94.40%): ¹H NMR (400 MHz, CDCl₃) δ 7.60 (s, 1H), 7.55 (d, J=8.8 Hz, 1H), 7.06-6.93 (m, 3H), 6.86-6.81 (m, 1H), 5.44 (br s, 1H), 3.15 (s, 3H), 2.78 (m, 1H), 2.58-2.46 (m, 1H), 2.39 (s, 3H), 2.35-2.07 (m, 12H), 1.84-1.81 (m, 6H), 1.73-1.68 (m, 1H); LCMS: MS (ESI) m/z (M+H)+: 561.4.

Analysis method: Chiralpak OD-3 100×4.6 mm I.D., 3 μm; mobile phase: A: supercritical carbon dioxide, B: 0.05% diethylamine in ethanol; gradient: from 5% B to 40% B in 4 minutes, 40% B for 2.5 min, from 5% B back to 1.5% B in 1.5 min; flow rate: 2.8 mL/min; column temperature: 35° C.; wavelength: 220 nm.

### EXAMPLE 8

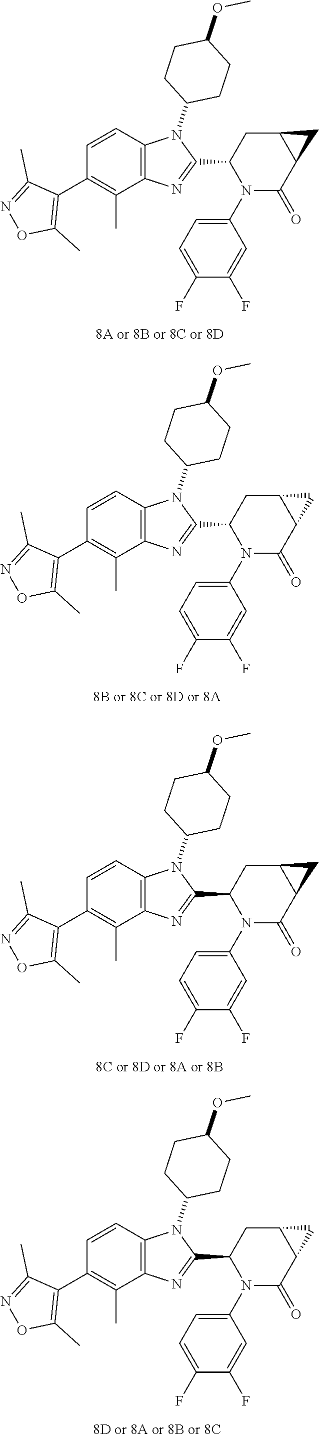

Route of Synthesis:

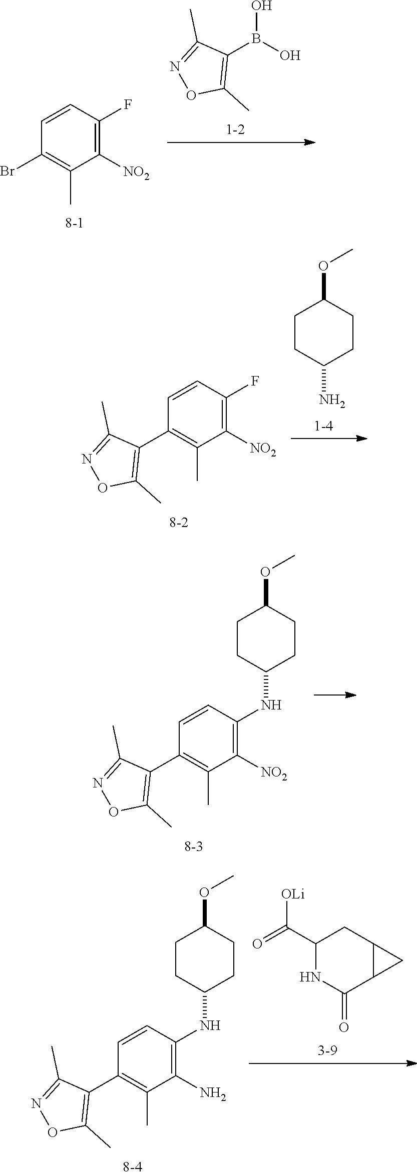

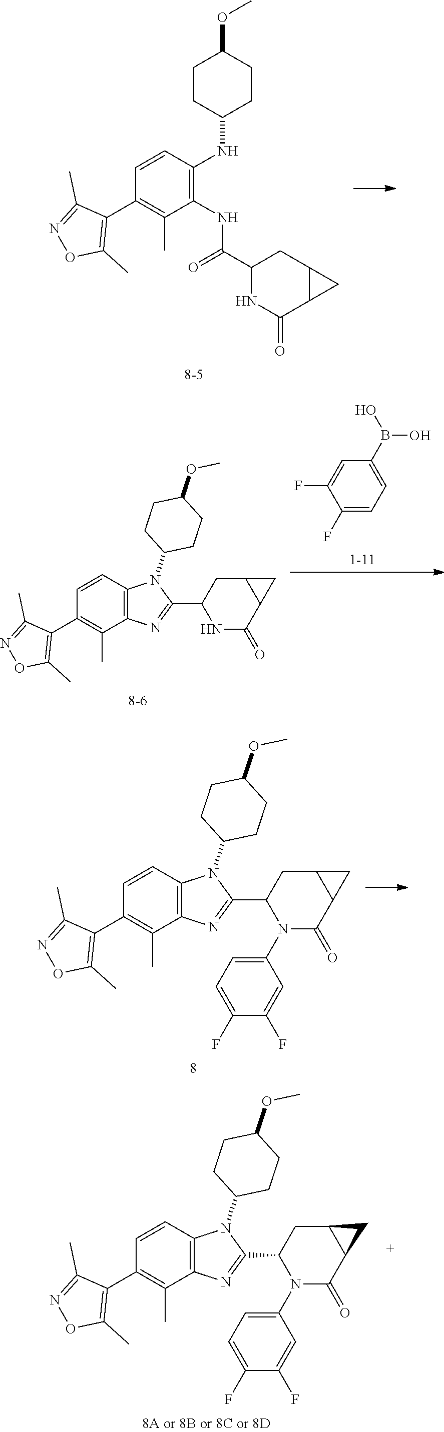

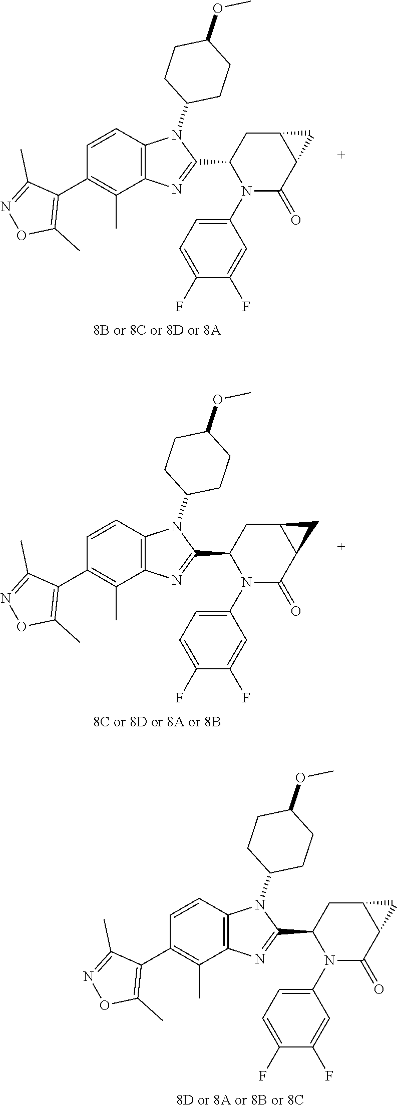

### Preparation of Compound 8-2

To a microwave tube were added compound 8-1 (900 mg, 3.85 mmol, 2.79 mL, 1 eq), compound 1-2 (810 mg, 5.75 mmol, 1.49 eq), potassium carbonate (1.60 g, 11.59 mmol, 3.01 eq), 1,4-dioxane (10 mL), water (2 mL) and Pd(dppf)Cl₂ (282 mg, 385.40 μmol, 0.1 eq). The reaction solution was purged with nitrogen, heated to 100° C. with microwave, and reacted for 0.5 hours. The reaction solution was filtered through Celite, and the filtrate was concentrated under reduced pressure. To the residue were added ethyl acetate (30 mL) and saturated brine (30 mL). The layers were separated. The organic phase was dried over anhydrous sodium sulfate, and filtered. The filtrate was concentrated under reduced pressure. The residue was purified by column chromatography (petroleum ether/ethyl acetate=1/0˜3/1) to give compound 8-2. LCMS: MS (ESI) m/z (M+H)+: 251.1.

### Preparation of Compound 8-3

To a microwave tube were added compound 8-2 (380 mg, 1.52 mmol, 1 eq), N,N-dimethylformamide (10 mL), Cs₂CO₃ (1.48 g, 4.55 mmol, 3 eq) and compound 1-4 (380 mg, 2.29 mmol, 1.51 eq), and the resulting reaction solution was heated to 120° C. and reacted with microwave for 1 hour. The reaction solution was filtered, and the filtrate was concentrated under reduced pressure. The residue was purified by column chromatography(petroleum ether/ethyl acetate=3/1˜1/1) to give compound 8-3. LCMS: MS (ESI) m/z (M+H)+: 360.3.

### Preparation of Compound 8-4

To a solution of sodium hydrosulfite (1.27 g, 7.27 mmol, 1.58 mL, 10.05 eq), aqueous ammonia (14.54 mmol, 2 mL, 20.10 eq), tetrahydrofuran (4 mL) and water (4 mL) was added compound 8-3 (260 mg, 723.39 μmol, 1 eq), and the resulting reaction solution was stirred at 15° C. for 16 hours. The reaction solution was poured into water (15 mL), and the mixture was extracted with ethyl acetate (20 mL*2). The organic phase was washed with saturated brine (15 mL), dried over anhydrous sodium sulfate, and filtered. The filtrate was concentrated under reduced pressure to give compound 8-4. LCMS: MS (ESI) m/z (M+H)+: 330.3.

### Preparation of Compound 8-5

To a solution of compound 3-9 (480 mg, 3.09 mmol, 4.25 eq), acetonitrile (5 mL) and N,N-dimethylformamide (1 mL) were added N-methylimidazole (288 mg, 3.51 mmol, 279.61 μL, 4.82 eq), compound 8-4 (240 mg, 728.52 μmol, 1 eq) and HATU (456 mg, 1.63 mmol, 2.23 eq), and the resulting reaction solution was stirred at 10° C. for 16 hours. To the reaction solution was added additional compound 3-9 (480 mg, 3.09 mmol, 4.25 eq), and the reaction solution was stirred at 10° C. for another 18 hours. The reaction solution was poured into water (30 mL). The mixture was extracted with ethyl acetate (30 mL*2), and the layers were separated. The organic phase was washed sequentially with saturated sodium bicarbonate solution (30 mL) and saturated brine (30 mL), dried over anhydrous sodium sulfate, and filtered. The filtrate was concentrated under reduced pressure. The residue was purified by column chromatography (dichloromethane/tetrahydrofuran=1/0˜0/1) to give compound 8-5. LCMS: MS (ESI) m/z (M+H)+: 467.3.

### Preparation of Compound 8-6

To a microwave tube were added compound 8-5 (70 mg, 150.03 μmol, 1 eq) and acetic acid (2 mL), and the resulting reaction solution was heated to 100° C. and reacted with microwave for 0.5 hours. The reaction solution was concentrated under reduced pressure. To the residue was added dichloromethane (20 mL). The mixture was washed with saturated sodium bicarbonate solution (20 mL), dried over anhydrous sodium sulfate, and filtered. The filtrate was concentrated under reduced pressure to give compound 8-6. LCMS: MS (ESI) m/z (M+H)+: 449.4.

### Preparation of Compounds 8A, 8B and 8C

To a solution of compound 8-6 (90 mg, 200.64 μmol, 1 eq) and methanol (2 mL) was added DBU (72 mg, 472.94 μmol, 71.29 μL, 2.36 eq), and the mixture was stirred for 15 minutes. CU-TMEDA CATALYST (II) (20 mg, 43.06 μmol, 2.15e-1 eq) was added, and the mixture was stirred for 15 minutes. Compound 1-11 (130 mg, 823.25 μmol, 4.10 eq) was added, and the resulting reaction solution was stirred at 10° C. for 16 hours. The reaction solution was filtered, and the filtrate was concentrated under reduced pressure. The residue was purified by column chromatography (dichloromethane/tetrahydrofuran=5/1˜0/1) to give a product. The product was analyzed to be racemic compound 8 (racemization was presumed to have occurred during the preparation of 8-5 or 8-6 or compound 8) by detection with supercritical fluid chromatography (Chiralpak IC-3 100×4.6 mm I.D., 3 μm; Mobile phase: A: supercritical carbon dioxide, B: 0.05% diethylamine in ethanol; gradient: 40% B; flow rate: 2.8 mL/min; column temperature: 35° C.; wavelength: 220 nm). Compound 8 was isolated by SFC (column: DAICEL CHIRALPAK AS (250 mm*30 mm, 10 μm); mobile phase: [0.1% NH₃H₂O EtOH]; 30%-30%, min) to give chiral isomers compound 8A, compound 8B and compound 8C.

Compound 8A (retention time was 2.463 min, ee=100%): ¹H NMR (400 MHz, CDCl₃) δ 7.16 (br s, 1H), 7.02 (brs, 1H), 6.96-6.80 (m, 3H), 5.05-5.49 (m, 1H), 4.00 (brs, 1H), 3.34 (brs, 3H), 3.22-3.18 (m, 1H), 2.71-2.66 (m, 1H), 2.35 (s, 4H), 2.18-2.15 (m, 7H), 2.07-1.96 (m, 4H), 1.65 (br s, 1H), 1.43-1.19 (m, 6H); LCMS: MS (ESI) m/z (M+H)+: 561.4.

Compound 8B (retention time was 3.443 min, ee=98.34%): ¹H NMR (400 MHz, CDCl₃) δ 7.17-7.10 (m, 1H), 7.02 (br s, 1H), 6.86-6.81 (m, 3H), 5.05-5.47 (m, 1H), 4.00 (brs, 1H), 3.34 (br s, 3H), 3.22-3.17 (m, 1H), 2.71-2.65 (m, 1H), 2.35 (br s, 4H), 2.18-2.13 (m, 7H), 2.04 (br s, 3H), 1.64 (br s, 2H), 1.42-1.22 (m, 5H), 0.77 (br s, 1H); LCMS: MS (ESI) m/z (M+H)+: 561.4.

Compound 8C (retention time was 5.808, ee=100%): LCMS: MS (ESI) m/z (M+H)+: 561.3.

Analysis method: Chiralpak AS-3 100×4.6 mm I.D., 3 μm, mobile phase: A: supercritical carbon dioxide, B: 0.05% diethylamine in ethanol; gradient: from 5% B to 40% B in 4 minutes, 40% B for 2.5 min, from 5% B back to 1.5% B in 1.5 min; flow rate: 2.8 mL/min; column temperature: 35° C.; wavelength: 220 nm.

### EXAMPLE 9

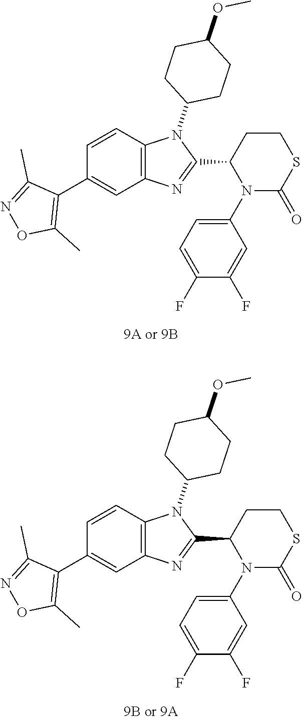

Route of Synthesis:

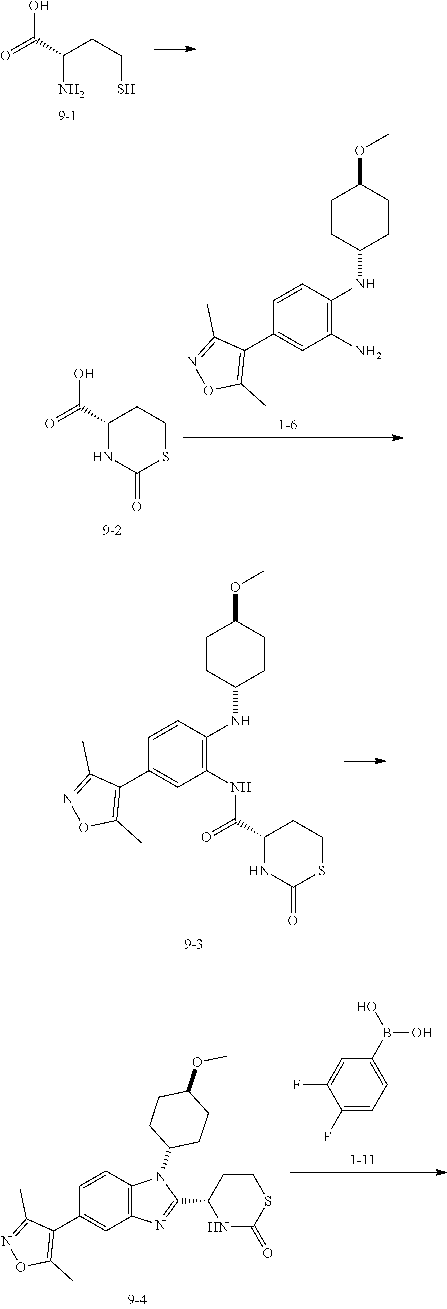

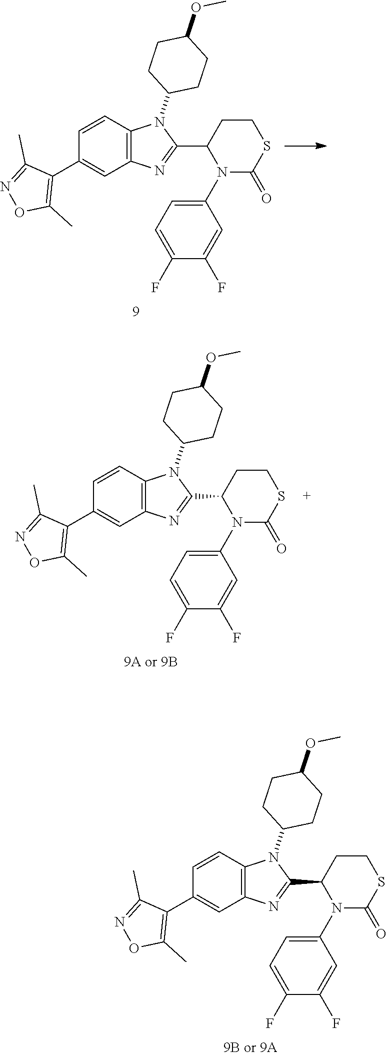

### Preparation of Compound 9-2

To a mixture of compound 9-1(0.1 g, 739.73 μmol, 1 eq), sodium hydroxide (180 mg, 4.50 mmol, 6.08 eq) and water (2.5 mL) at 0° C. was added a solution of triphosgene (220 mg, 741.37 μmol, 1.00 eq) in 1,4-dioxane (1.2 mL) dropwise (kept at 0˜5° C.), and the resulting reaction solution was slowly warmed to 15° C. and stirred for 40 hours. The reaction solution was concentrated under reduced pressure, and acetonitrile (5 mL) was added. The mixture was heated to 60° C. and stirred for 0.5 hours, and filtered while hot. The filtrate was concentrated under reduced pressure to give compound 9-2. LCMS: MS (ESI) m/z (M+H)+: 162.0.

### Preparation of Compound 9-3

To a solution of compound 9-2 (100 mg, 620.43 μmol, 1.80 eq), N,N-dimethylformamide (2 mL) and acetonitrile (2 mL) were added N-methylimidazole (88 mg, 1.07 mmol, 85.44 μL, 3.10 eq), compound 1-6 (109 mg, 345.58μmol, 1 eq) and N,N,N′,N′-tetramethylchloroformamidinium hexafluorophosphate (175 mg, 623.71 μmol, 1.8 eq), and the resulting reaction solution was stirred at 15° C. for 16 hours. To the reaction solution were added water (20 mL) and ethyl acetate (30 mL), and the layers were separated. The organic phase was washed sequentially with IN hydrochloric acid solution (20 mL), saturated sodium bicarbonate solution (20 mL) and saturated brine (20 mL), dried over anhydrous sodium sulfate, and filtered. The filtrate was concentrated under reduced pressure. The residue was purified by column chromatography (dichloromethane/tetrahydrofuran=1/0˜0/1) to give compound 9-3. LCMS: MS (ESI) m/z (M+H)+: 459.3.

### Preparation of Compound 9-4

A solution of compound 9-3 (160 mg, 348.91 μmol, 1 eq) and acetic acid (3 mL) was heated to 80° C. and stirred for 16 hours. The reaction solution was concentrated under reduced pressure. To the residue was added dichloromethane (20 mL). The mixture was washed with saturated sodium bicarbonate solution (20 mL), dried over anhydrous sodium sulfate, and filtered. The filtrate was concentrated under reduced pressure, and the residue was purified by column chromatography (dichloromethane/tetrahydrofuran=1/0˜0/1) to give compound 9-4. LCMS: MS (ESI) m/z (M+H)+: 441.1.

### Preparation of Compounds 9A and 9B

To a solution of compound 9-4 (140 mg, 317.78 μmol, 1 eq) in methanol (5 mL) was added DBU (110 mg, 722.54 μmol, 108.91 μL, 2.27 eq) at 15° C., and the mixture was stirred for 15 minutes. CU-TMEDA CATALYST (II) (30 mg, 64.60 μmol, 2.03e-1 eq) was added, and the mixture was stirred for 15 minutes. Compound 1-11 (200 mg, 1.27 mmol, 3.99 eq) was added, and the resulting reaction solution was stirred at 15° C. for 16 hours. The reaction solution was filtered, and the filtrate was concentrated under reduced pressure. The residue was purified by column chromatography (dichloromethane/tetrahydrofuran=5/1˜0/1) to give a product. The product was analyzed to be racemic compound 9 (racemization was presumed to have occurred during the preparation of 9-3 or 9-4 or compound 9) by detection with supercritical fluid chromatography (Chiralpak AS-3 100×4.6 mm I.D., 3 μm; mobile phase: A: supercritical carbon dioxide, B: 0.05% diethylamine in ethanol; gradient: from 5% B to 40% B in 4 minutes, 40% B for 2.5 min, from 5% B back to 1.5% B in 1.5 min; flow rate: 2.8 mL/min; column temperature: 35° C.; wavelength: 220 nm). Compound 9 was isolated by SFC (column: DAICEL CHIRALPAK AS (250 mm*30 mm, 10 μm); mobile phase: [0.1% NH₃H₂O EtOH]; 25%-25%) to give chiral isomers compound 9A and compound 9B.

Compound 9A (retention time was 3.012 min, ee=100%): ¹H NMR (400 MHz, CDCl₃) δ 7.70 (s, 1H), 7.44 (d, J=8.8 Hz, 1H), 7.10-7.03 (m, 2H), 7.03-6.96 (m, 1H), 6.93-6.87 (m Hz, 1H), 5.25 (s, 1H), 3.91 (br s, 1H), 3.49-3.39 (m, 1H), 3.33 (s, 3H), 3.23-3.17 (m, 1H), 2.91-2.82 (m, 1H), 2.79-2.68 (m, 1H), 2.39 (s, 4H), 2.34-2.10 (m, 8H), 1.80-1.75 (m, 1H), 1.36-1.28 (m, 2H); LCMS: MS (ESI) m/z (M+H)+: 553.3.

Compound 9B (retention time was 3.502 min, ee=100%): ¹H NMR (400 MHz, CDCl₃) δ 7.69 (s, 1H), 7.44 (d, J=8.8 Hz, 1H), 7.09-7.04 (m, 2H), 7.02-6.97 (m, 1H), 6.93-6.81 (m, 1H), 5.25 (s, 1H), 3.91 (br s, 1H), 3.49-3.39 (m, 1H), 3.33 (s, 3H), 3.28-3.17 (m, 1H), 2.91-2.87 (m, 1H), 2.79-2.69 (m, 1H), 2.39 (s, 4H), 2.30-2.12 (m, 8H), 1.80-1.75 (m, 1H), 1.37-1.31 (m, 2H); LCMS: MS (ESI) m/z (M+H)+: 553.3.

Analysis method: Chiralpak AS-3 100×4.6 mm I.D., 3 μm; mobile phase: A: supercritical carbon dioxide, B: 0.05% diethylamine in ethanol; gradient: from 5% B to 40% B in 4 minutes, 40% B for 2.5 min, from 5% B back to 1.5% B in 1.5 min; flow rate: 2.8 mL/min; column temperature: 35° C.; wavelength: 220 nm.

### EXAMPLE 10

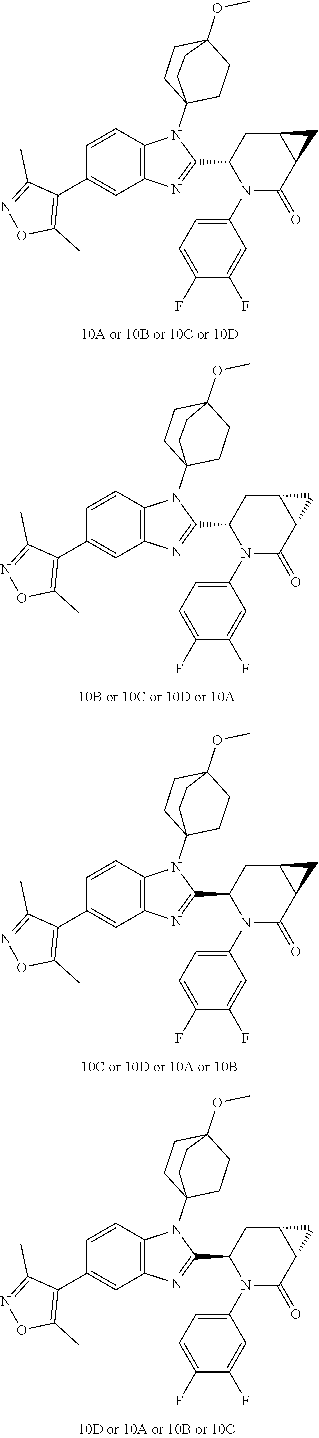 [SMILES: COC12CCC(n3c([C@@H]4C[C@H]5C[C@H]5C(=O)N4c4ccc(F)c(F)c4)nc4cc(-c5c(C)noc5C)ccc43)(CC1)CC2 ; COC12CCC(n3c([C@@H]4C[C@@H]5C[C@@H]5C(=O)N4c4ccc(F)c(F)c4)nc4cc(-c5c(C)noc5C)ccc43)(CC1)CC2 ; COC12CCC(n3c([C@H]4C[C@H]5C[C@H]5C(=O)N4c4ccc(F)c(F)c4)nc4cc(-c5c(C)noc5C)ccc43)(CC1)CC2 ; COC12CCC(n3c([C@H]4C[C@@H]5C[C@@H]5C(=O)N4c4ccc(F)c(F)c4)nc4cc(-c5c(C)noc5C)ccc43)(CC1)CC2]

Route of Synthesis:

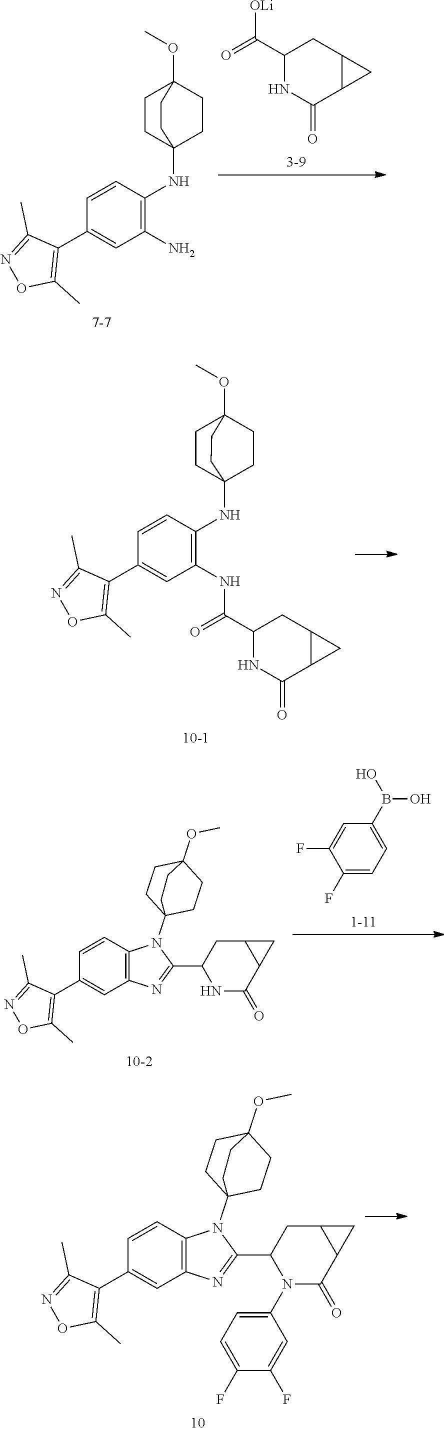

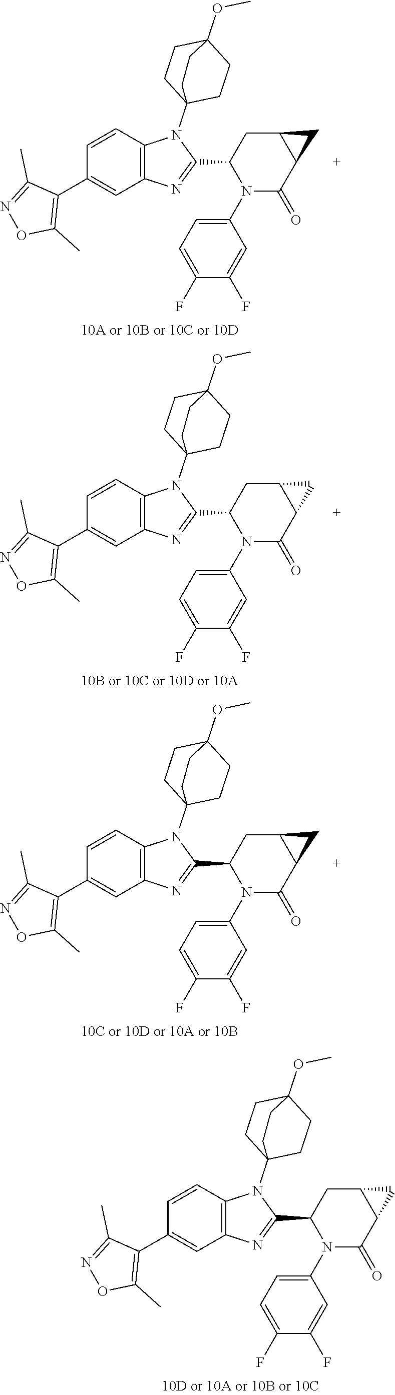

### Preparation of Compound 10-1

To a solution of compound 3-9 (165 mg, 1.06 mmol, 3.03 eq), acetonitrile (5 mL) and N,N-dimethylformamide (2 mL) were added 1-methylimidazole (145 mg, 1.77 mmol, 140.78 μL, 5.03 eq), compound 7-7 (120 mg, 351.45 μmol, 1 eq) and HATU (222 mg, 791.23 μmol, 2.25 eq), and the resulting reaction solution was stirred at 15° C. for 2 hours. The reaction solution was poured into water (10 mL). The mixture was extracted with ethyl acetate (20 mL*2), and the layers were separated. The organic phase was washed sequentially with saturated sodium bicarbonate solution (20 mL) and saturated brine (30 mL), dried over anhydrous sodium sulfate, and filtered. The filtrate was concentrated under reduced pressure. The residue was purified by column chromatography (petroleum ether/tetrahydrofuran=1/0˜0/1) to give compound 10-1. LCMS: MS (ESI) m/z (M+H)+: 479.3.

### Preparation of Compound 10-2

A solution of compound 10-1 (200 mg, 417.90 μmol, 1 eq) and acetic acid (3 mL) was heated to 110° C. and stirred for 16 hours. The reaction solution was concentrated under reduced pressure. To the residue was added dichloromethane (30 mL). The mixture was washed with saturated sodium bicarbonate solution (30 mL), dried over anhydrous sodium sulfate, and filtered. The filtrate was concentrated under reduced pressure. The residue was purified by column chromatography (petroleum ether/tetrahydrofuran=5/10/1) to give compound 10-2. LCMS: MS (ESI) m/z (M+H)+: 461.3.

### Preparation of Compounds 10A and 10B

To a solution of compound 10-2 (130 mg, 282.26 μmol, 1 eq), compound 1-11 (130 mg, 823.25 μmol, 2.92 eq) and dichloromethane (5 mL) were added pyridine (245 mg, 3.10 mmol, 0.25 mL, 10.97 eq) and copper acetate monohydrate (65 mg, 325.57 μmol, 65.00 μL, 1.15 eq) at 15° C., and the resulting reaction solution was stirred at 15° C. for 16 hours. To the reaction solution was added saturated ammonium chloride solution (15 mL) and dichloromethane (30 mL), and the layers were separated. The organic phase was washed sequentially with 1M hydrochloric acid solution (pH of about 2), saturated sodium bicarbonate solution (20 mL) and saturated brine (20 mL), dried over anhydrous sodium sulfate, and filtered. The filtrate was concentrated under reduced pressure. The residue was purified by column chromatography (petroleum ether/tetrahydrofuran=5/10/1) to give a product. The product was analyzed to be racemic compound 10 (racemization was presumed to have occurred during the preparation of 10-1 or 10-2 or compound 10) by detection with supercritical fluid chromatography (Chiralpak AS-3 100×4.6 mm I.D., 3 μm; mobile phase: A: supercritical carbon dioxide, B: 0.05% diethylamine in ethanol; gradient: from 5% B to 40% B in 4 minutes, 40% B for 2.5 min, from 5% B back to 1.5% B in 1.5 min; flow rate: 2.8 mL/min; column temperature: 35° C.; wavelength: 220 nm). Compound 10 was isolated by SFC (column: DAICEL CHIRALCEL OD-H (250 mm*30 mm, 5 μm); mobile phase: [0.1% NH₃H₂O EtOH]; 45%-45%) to give chiral isomers compound 10A and compound 10B. Compound 10A (retention time was 3.004 min, ee=100%): ¹H NMR (400 MHz, CDCl₃) δ 7.56 (s, 1H), 7.51-7.41 (m, 1H), 7.21-7.15 (m, 1H), 6.95-6.83 (m, 2H), 6.78-6.72 (m, 1H), 5.21-5.10 (m, 1H), 3.16 (s, 3H), 2.82-2.69 (m, 1H), 2.40-2.28 (m, 7H), 2.26-2.19 (m, 6H), 2.05-1.97 (m, 1H), 1.86-1.85 (m, 6H), 1.27-1.12 (m, 2H), 1.00-0.93 (m, 1H); LCMS: MS (ESI) m/z (M+H)+: 573.4.

Compound 10B (retention time was 3.959 min, ee=93.98%): ¹H NMR (400 MHz, CDCl₃) δ 7.56 (s, 1H), 7.51-7.49 (m, 1H), 7.22-7.13 (m, 1H), 6.95-6.81 (m, 2H), 6.75-6.71 (m, 1H), 5.21-5.14 (m, 1H), 3.16 (s, 3H), 2.82-2.69 (m, 1H), 2.42-2.28 (m, 7H), 2.24 (s, 6H), 2.01-1.98 (m, 1H), 1.86-1.84 (m, 6H), 1.34-1.09 (m, 2H), 1.00-0.95 (m, 1H); LCMS: MS (ESI) m/z (M+H)+: 573.3.

Analysis method: Chiralpak AS-3 100×4.6 mm I.D., 3 μm; mobile phase: A: supercritical carbon dioxide, B: 0.05% diethylamine in ethanol; gradient: from 5% B to 40% B in 4 minutes, 40% B for 2.5 min, from 5% B back to 1.5% B in 1.5 min; flow rate: 2.8 mL/min; column temperature: 35° C.; wavelength: 220 nm.

### EXAMPLE 11

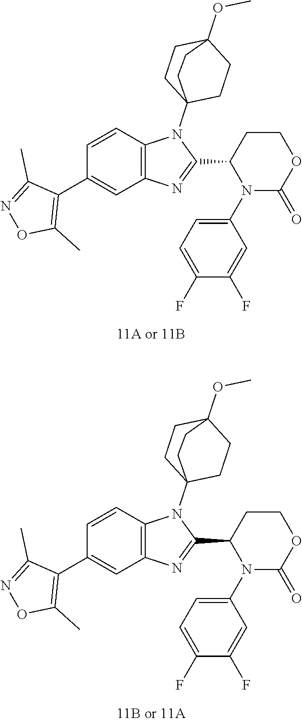 [SMILES: COC12CCC(n3c([C@@H]4CCOC(=O)N4c4ccc(F)c(F)c4)nc4cc(-c5c(C)noc5C)ccc43)(CC1)CC2 ; COC12CCC(n3c([C@H]4CCOC(=O)N4c4ccc(F)c(F)c4)nc4cc(-c5c(C)noc5C)ccc43)(CC1)CC2]

Route of Synthesis:

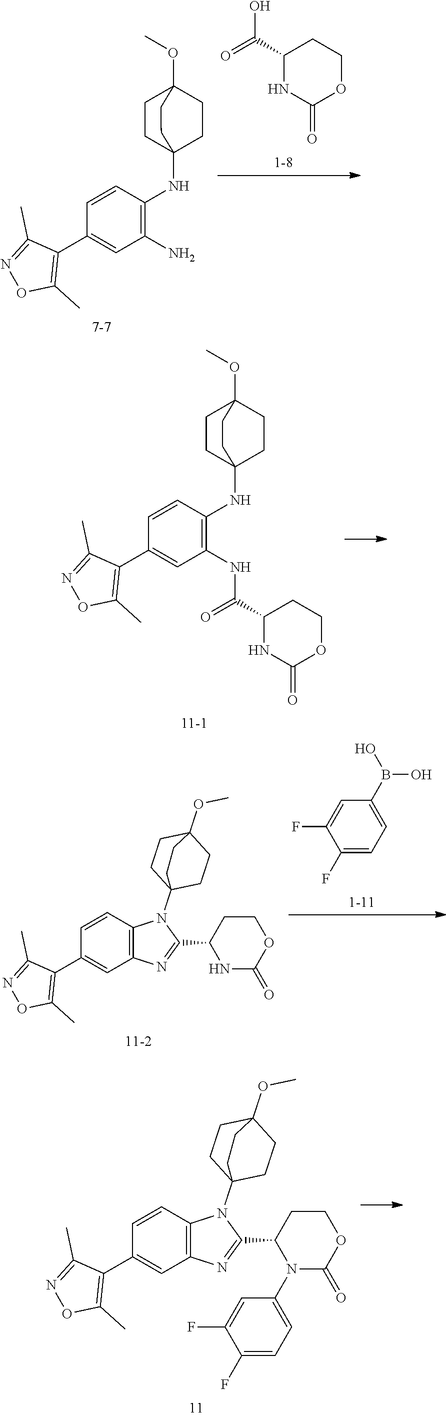 [SMILES: COC12CCC(Nc3ccc(-c4c(C)noc4C)cc3N)(CC1)CC2 ; O=C1N[C@H](C(=O)O)CCO1 ; COC12CCC(Nc3ccc(-c4c(C)noc4C)cc3NC(=O)[C@@H]3CCOC(=O)N3)(CC1)CC2 ; COC12CCC(n3c([C@@H]4CCOC(=O)N4)nc4cc(-c5c(C)noc5C)ccc43)(CC1)CC2 ; OB(O)c1ccc(F)c(F)c1 ; COC12CCC(n3c([C@@H]4CCOC(=O)N4c4ccc(F)c(F)c4)nc4cc(-c5c(C)noc5C)ccc43)(CC1)CC2]

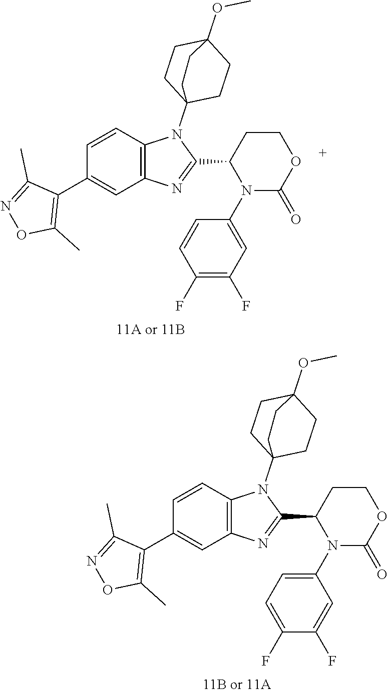 [SMILES: COC12CCC(n3c([C@@H]4CCOC(=O)N4c4ccc(F)c(F)c4)nc4cc(-c5c(C)noc5C)ccc43)(CC1)CC2 ; COC12CCC(n3c([C@H]4CCOC(=O)N4c4ccc(F)c(F)c4)nc4cc(-c5c(C)noc5C)ccc43)(CC1)CC2]

### Preparation of Compound 11-1

To a solution of compound 7-7 (120 mg, 826.94 μmol, 2.35 eq), acetonitrile (5 mL) and N,N-dimethylformamide (2 mL) were added 1-methylimidazole (145 mg, 1.77 mmol, 140.78 μL, 5.03 eq), compound 1-8 (120 mg, 351.45 μmol, 1 eq) and N,N,N′,N′-tetramethylchloroformamidinium hexafluorophosphate (222 mg, 791.22 μmol, 2.25 eq), and the resulting reaction solution was stirred at 15° C. for 2 hours. The reaction solution was poured into water (10 mL). The mixture was extracted with ethyl acetate (15 mL*2), and the layers were separated. The organic phase was washed sequentially with saturated sodium bicarbonate solution (10 mL) and saturated brine (20 mL), dried over anhydrous sodium sulfate, and filtered. The filtrate was concentrated under reduced pressure. The residue was purified by column chromatography (petroleum ether/tetrahydrofuran=1/0˜0/1) to give compound 11-1. LCMS: MS (ESI) m/z (M+H)+: 469.2.

### Preparation of Compound 11-2

A solution of compound 11-1 (140 mg, 298.80 μmol, 1 eq) and acetic acid (3 mL) was heated to 110° C. and stirred for 16 hours. The reaction solution was concentrated under reduced pressure. To the residue was added dichloromethane (30 mL). The mixture was washed with saturated sodium bicarbonate solution (30 mL), dried over anhydrous sodium sulfate, and filtered. The filtrate was concentrated under reduced pressure. The residue was purified by column chromatography (petroleum ether/tetrahydrofuran=5/1˜0/1) to give compound 11-2. LCMS: MS (ESI) m/z (M+H)+: 451.4.

### Preparation of Compounds 11A and 11B

To a solution of compound 11-2 (100 mg, 221.96 μmol, 1 eq), compound 1-11 (150 mg, 949.91 μmol, 4.28 eq) and dichloromethane (5 mL) were added pyridine (176 mg, 2.23 mmol, 179.59 μL, 10.02 eq) and copper acetate (50 mg, 275.28 μmol, 1.24 eq) at 15° C., and the resulting reaction solution was stirred at 15° C. for 16 hours. To the reaction solution were added additional compound 1-11 (150 mg, 949.91 μmol, 4.28 eq) and copper acetate (50 mg, 275.28 μmol, 1.24 eq), and the resulting reaction solution was stirred at 15° C. for another 16 hours. To the reaction solution were added saturated ammonium chloride solution (15 mL) and dichloromethane (30 mL), and the layers were separated. The organic phase was washed sequentially with 1M hydrochloric acid solution, saturated sodium bicarbonate solution (20 mL) and saturated brine (20 mL), dried over anhydrous sodium sulfate, and filtered. The filtrate was concentrated under reduced pressure. The residue was purified by column chromatography (petroleum ether/tetrahydrofuran=5/1˜0/1) to give a product. The product was analyzed to be racemic compound 11 (racemization was presumed to have occurred during the preparation of 11-1 or 11-2 or compound 11) by detection with supercritical fluid chromatography (Chiralpak OD-3 100×4.6 mm I.D., 3 μm; mobile phase: A: supercritical carbon dioxide, B: 0.05% diethylamine in ethanol; gradient: from 5% B to 40% B in 4 minutes, 40% B for 2.5 min, from 5% B back to 1.5% B in 1.5 min; flow rate: 2.8 mL/min; column temperature: 35° C.; wavelength: 220 nm). Compound 11 was isolated by SFC (column: DAICEL CHIRALCEL OD-H (250 mm*30 mm, 5 μm); mobile phase: [0.1% NH₃H₂O EtOH]; 45%-45%) to give chiral isomers compound 11A and compound 11B.

Compound 11A (retention time was 4.258 min, ee=99.36%): ¹H NMR (400 MHz, CDCl₃) δ 7.63 (s, 1H), 7.58-7.56 (m, 1H), 7.11-6.91 (m, 4H), 5.46-4.41 (m, 1H), 4.83-7.80 (m, 1H), 4.28-4.25 (m, 1H), 3.15 (s, 3H), 2.75-2.64 (m, 1H), 2.39 (s, 3H), 2.28-2.14 (m, 10H), 1.85-1.81 (m, 6H); LCMS: MS (ESI) m/z (M+H)+: 563.4.

Compound 11B (retention time was 4.887 min, ee=100%): ¹H NMR (400 MHz, CDCl₃) δ 7.63 (s, 1H), 7.58-7.51 (m, 1H), 7.14-6.86 (m, 4H), 5.46-5.40 (m, 1H), 4.83-4.78 (m, 1H), 4.28-4.21 (m, 1H), 3.15 (s, 3H), 2.70-2.65 (m, 1H), 2.39 (s, 3H), 2.26 (br s, 9H), 2.03-2.19 (m, 1H), 1.85-1.74 (m, 6H); LCMS: MS (ESI) m/z (M+H)+: 563.3.

Analysis method: Chiralpak OD-3 100×4.6 mm I.D., 3 μm; mobile phase: A: supercritical carbon dioxide, B: 0.05% diethylamine in ethanol; gradient: from 5% B to 40% B in 4 minutes, 40% B for 2.5 min, from 5% B back to 1.5% B in 1.5 min; flow rate: 2.8 mL/min; column temperature: 35° C.; wavelength: 220 nm.

### Assay Example 1: Assay of p300 and CBP Protein Activity

Reagent:

Reaction buffer: 25 mM HEPES, pH 7.5, 25 mM NaCl, 0.025% CHAPS, 0.025% BSA, 0.5% DMSO.

Ligand:

Histone H₄ (1-21) K5/8/12/16Ac-GG-Biotin.

Standard Reaction Conditions:

5 nM p300-GST, 50 nM peptide ligand.

Reaction Procedure:
1. 2.5×BRD was added to the wells of a reaction plate, except for control wells without BRD to which buffer was added instead.
2. Compounds in 100% DMSO were delivered into the BRD mixture by acoustic technology (Echo550; nanoliter range). The plate was spun down. The mixture was incubated at room temperature for 30 min.
3. 5×peptide ligand was delivered. The plate was spun down. The mixture was incubated at room temperature for 10 min.
4. 5×HTRF detection mixture was delivered. The plate was spun down and the mixture was incubated in the dark for 2 h.
5. A measurement of HTRF was performed with an Envision reader (Ex/Em=320/615, 665 nm).
6. A HTRF ratio was calculated as follows: [em 665 nm/em 615 nm]*10000.

Data Analysis:

The background-subtracted signal (no protein as background) was converted to % bound relative to the DMSO control. Data were analyzed using GraphPad Prism 4 with “sigmoidal dose-response (variable slope)”; and a 4-parameter analysis was performed using Hill slope.

Constraints:
Bottom=Constant equal to 0
Top=Must be less than 120

TABLE 1
Data of p300 and CBP protein activity

| Compound No. | p300 (IC₅₀, nM) | CBP (IC₅₀, nM) |
|---|---|---|
| 2B | 3.54 | 0.67 |
| 3B | 2.88 | 0.67 |
| 3C | 14.1 | — |
| 4A | 11.3 | — |
| 6B | 2.09 | — |
| 7B | 4.11 | — |
| 8C | 10.3 | — |
| 9B | 11 | — |
| 10B | 4.08 | 3.13 |
| 11B | 5.48 | — |

Note:
“—” means no detection.

Conclusion: The compounds of the present disclosure exhibited good inhibitory activity on p300/CBP.

### Assay Example 2: Pharmacokinetic Study of Compounds of the Present Disclosure

Materials of the Assay:

CD-1 mice (male, 5-6 weeks old, Shanghai Family Planning Research Institute)

Procedure of the Assay:

Injection: To assay pharmacokinetic characteristics of rodents after intravenous injection administration of compounds by a standard protocol. In the assay, candidate compounds were formulated into clear solutions, and administered to mice as a single intravenous injection. The vehicle for the intravenous injection was a mixed vehicle formulated with 5% of dimethyl sulfoxide and 95% of 10% hydroxypropyl β-cyclodextrin. In this assay, two male CD-1 mice were used for intravenous injection administration at a dose of 0.5 mg/kg. Plasma samples were collected at 0.083, 0.25, 0.5, 1, 2, 4, 8 and 24 h after administration. Whole blood samples were collected within 24 hours, and centrifuged at 3000 g for 15 minutes. The supernatants were separated to obtain plasma samples. To the plasma samples was added 20 times volume of acetonitrile solution containing internal standard to precipitate protein. The mixtures were vortexed and centrifuged. The supernatants were taken for injection. Plasma drug concentration was quantitatively analyzed by LC-MS/MS analysis method, and pharmacokinetic parameters (such as clearance (CL), half-life (T_{1/2}), tissue distribution (Vdss), area under the drug-time curve (AUC₀₋ₗₐₛₜ), etc.) were calculated.

Oral: To assay pharmacokinetic characteristics of rodents after oral administration of compounds by a standard protocol. In the assay, candidate compounds were formulated into homogeneous opaque suspensions, and given to the mice for a single oral administration. The vehicle for the oral administration was a mixed vehicle formulated with 5% of dimethyl sulfoxide and 95% of 0.5% methylcellulose. In this assay, two male CD-1 mice were used for oral gavage administration at a dose of 3 mg/kg. Plasma samples were collected at 0.25, 0.5, 1, 2, 4, 8 and 24 h after administration. Whole blood samples were collected within 24 hours, and centrifuged at 3000 g for 15 minutes. The supernatants were separated to obtain plasma samples. To the plasma samples was added 20 times volume of acetonitrile solution containing internal standard to precipitate protein. The mixtures were vortexed and centrifuged. The supernatants were taken for injection. Plasma drug concentration was quantitatively analyzed by LC-MS/MS analysis method, and pharmacokinetic parameters (such as peak concentration (Cmax), half-life (T_{1/2}), area under the drug-time curve (AUC₀₋ₗₐₛₜ), etc.) were calculated.

TABLE 2
Summary of pharmacokinetic parameter data

| | | | | | | | Area | Area | |
|---|---|---|---|---|---|---|---|---|---|
| | | Plasma | Time | | Apparent | | under | under | |
| | | drug | to | half- | distribution | | curve | curve | |
| | | concentration | peak | life | volume | Clearance | (0-t) | (0-inf) | |
| Mode of | | Cₘₐₓ | Tmax | T_{1/2} | Vdss | Cl(mL/ | AUC₀₋ₗₐₛₜ | AUC_{0-inf} | Bioavailability |
| administration | Dosage | (nM) | (h) | (h) | (L/kg) | min/kg) | (nM.h) | (nM.h) | F (%) |
| Intravenous | 0.5 | — | — | 0.99 | 1.65 | 23.3 | 665 | 676 | — |
| administration | mg/kg | | | | | | | | |
| of 3B | | | | | | | | | |
| Gavage | 3 | 697 | 0.75 | 1.12 | — | — | 2577 | 2606 | 64% |
| administration | mg/kg | | | | | | | | |
| of 3B | | | | | | | | | |
| Intravenous | 0.5 | — | — | 0.78 | 1.46 | 22.8 | 626 | 652 | — |
| administration | mg/kg | | | | | | | | |
| of 7B | | | | | | | | | |
| Gavage | 3 | 1275 | 0.5 | 0.83 | — | — | 1588 | 1598 | 40% |
| administration | mg/kg | | | | | | | | |
| of 7B | | | | | | | | | |
| Intravenous | 0.5 | — | — | 0.732 | 1.33 | 22.9 | 624 | 637 | |
| administration | mg/kg | | | | | | | | |
| of 10B | | | | | | | | | |
| Gavage | 3 | 546 | 0.75 | 1.68 | | | 2019 | 2070 | 54% |
| administration | mg/kg | | | | | | | | |
| of 10B | | | | | | | | | |

Note:
“—”: None;
ND: no detection.

Conclusion: The compounds of the present disclosure had short half-life, wide distribution outside plasma, and moderate bioavailability.

### Assay Example 3: Assay of hERG Potassium Ion Channel Inhibition

1. Purpose of the assay:

The effect of the compounds to be assayed on hERG potassium ion channel was detected by the method of automatic patch clamp.

2. Method of the assay

2.1. Preparation of cells

2.1.1 CHO-hERG cells were cultured in a 175 cm² culture flask. When the cells grew to a density of 60-80%, the culture medium was removed. The cells were washed once with 7 mL of PBS (Phosphate Buffered Saline), and then digested by adding 3 mL of Detachin.

2.1.2 After the digestion was completed, 7 mL of culture medium was added for neutralization, and then the mixture was centrifuged. The supernatant was aspirated, and then 5 mL of culture medium was added for resuspension, so as to ensure that the cell density was 2×10⁶/mL˜5×10⁶/mL.

2.2. Formulation of solutions

Compositions of intracellular fluid and extracellular fluid were shown in Table 3.

TABLE 3
Compositions of intracellular and extracellular fluids

| Reagent | Extracellular fluid (mM) | Intracellular fluid (mM) |
|---|---|---|
| CaCl₂ | 1 | 1 |
| MgCl₂ | 1.25 | 1 |
| KCl | 5 | 140 |
| NaCl | 140 | 0 |
| Glucose | 10 | 0 |
| HEPES | 10 | 10 |
| EGTA | 0 | 10 |
| pH | Adjusted to a pH of 7.40 with | Adjusted to a pH of 7.20 with |
| | NaOH, | KOH, |
| | Osmotic pressure of ~305 | Osmotic pressure of ~290 mOsm |
| | mOsm | |

2.3 Recording process of electrophysiology

The single-cell high-impedance sealing and whole-cell mode formation processes were both completed automatically by a Qpatch instrument. After a whole-cell recording mode was achieved, the cells were clamped at −80 mV, given a front voltage of −50 millivolts for 50 milliseconds, followed by a depolarizing stimulus of +40 millivolts for 5 seconds, and then repolarized to −50 millivolts for 5 seconds, followed by a recovery to −80 millivolts. This voltage stimulation was applied every 15 s and recorded for 2 minutes. The extracellular fluid was then administered, followed by a record for 5 minutes. Then an administration process of compounds was started. The compound concentration assay was started from the lowest assay concentration, and each assay concentration was administrated for 2.5 minutes. After all concentrations were administrated continuously, 3 μM Cisapride was administered as a positive control compound. At least 3 cells (n≥3) were assayed for each concentration.

2.4. Preparation of compounds

2.4.1 A stock solution of compounds was diluted with DMSO. 10 μL of the stock solution of compounds was weighed and added to 20 μL of DMSO. Compounds were serially diluted 3-fold to 6 concentrations in DMSO.

2.4.2 4 μL of the compound with 6 concentrations in DMSO were weighed respectively, and added to 396 μL of an extracellular fluid. The mixtures were diluted 100-fold to 6 intermediate concentrations. Then 80 μL of the compound with 6 intermediate concentrations were weighed respectively, and added to 320 μL of an extracellular fluid. The mixtures were diluted 5-fold to the final concentration to be assayed.

2.4.3 The highest assay concentration was 40 μM, and there were 6 concentrations in total, which were 40, 13.3, 4.4, 1.48, 0.494, and 0.165 μM, respectively.

2.4.4 For the final assay concentration, the content of DMSO did not exceed 0.2%, and such concentration of DMSO had no effect on the hERG potassium channel.

2.4.5 The whole dilution process of the preparation of compounds was completed by a Bravo instrument.

2.5 Data analysis

The assay data were analyzed by GraphPad Prism 5.0 software.

2.6 Quality Control

Environment: humidity 20˜50%, temperature 22˜25° C.;

Reagents: The assay reagents used were purchased from Sigma, with a purity of >98%.

The assay data in the report must meet the following standards:
whole-cell sealing impedance >100 MΩ;
tail current magnitude >300 pA.
pharmacological parameters: the inhibitory effects of multiple concentrations of Cisapride on the hERG channel were set as a positive control.

2.7 Assay results: see Table 4.

TABLE 4
Results of IC₅₀ value of the example compounds against hERG

| Assay samples | hERG IC₅₀ (μM) | Number of assay |
|---|---|---|
| Example 7B | >40 | n = 3 |
| Example 10B | >40 | n = 3 |
| Cisapride | 0.036 | n = 3 |
| (positive control) | | |

1. A compound represented by formula (P) or a pharmaceutically acceptable salt thereof, 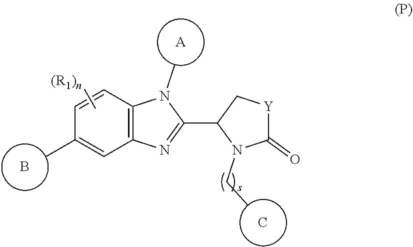
wherein
R₁ is selected from H, F, Cl, Br, I and C₁₋₃ alkyl, wherein the C₁₋₃ alkyl is optionally substituted by 1, 2 or 3 Rₐ;
n is selected from 1, 2 and 3;
s is selected from 0, 1 and 2;
Y is selected from —CH₂O—, —CH₂CH₂—, —N(R_{b})—, —CH₂S— and -cyclopropylidene;
ring A is selected from cyclohexyl, 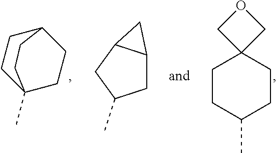
wherein the cyclohexyl, 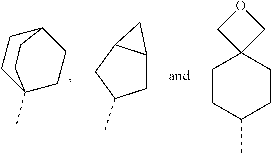
are optionally substituted by 1, 2 or 3 R₂;
ring B is selected from 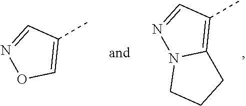
wherein the 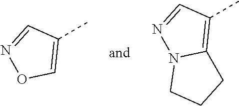
are optionally substituted by 1, 2 or 3 R₃;
ring C is selected from phenyl and 5-6 membered heteroaryl, wherein the phenyl and 5-6 membered heteroaryl are optionally substituted by 1, 2 or 3 R₄;
R₂, R₃ and R₄ are each independently selected from H, F, Cl, Br, I, OH, COOH, C₁₋₃ alkyl and C₁₋₃ alkoxy, wherein the C₁₋₃ alkyl and C₁₋₃ alkoxy are optionally substituted by 1, 2 or 3 R_{c};
Rₐ and R_{c} are each independently selected from F, Cl, Br, I and OH;
R_{b} is independently selected from H and CH₃;
provided that, when Y is selected from —CH₂CH₂— and —CH₂O—, ring A is not cyclohexyl.

2. The compound or a pharmaceutically acceptable salt thereof according to claim 1, wherein R₁ is selected from H, F, Cl, Br, I and CH₃, wherein the CH₃ is optionally substituted by 1, 2 or 3 Rₐ.

3. The compound or a pharmaceutically acceptable salt thereof according to claim 2, wherein R₁ is selected from H, F, Cl, Br, I, CH₃, CH₂F, CHF₂ and CF₃.

4. The compound or a pharmaceutically acceptable salt thereof according to claim 1, wherein R₂, R₃ and R₄ are each independently selected from H, F, Cl, Br, I, OH, CH₃ and OCH₃, wherein the CH₃ and OCH₃ are optionally substituted by 1, 2 or 3 R_{c}.

5. The compound or a pharmaceutically acceptable salt thereof according to claim 4, wherein R₂, R₃ and R₄ are each independently selected from H, F, Cl, Br, I, OH, CH₃, CH₂OH, CH₂F, CHF₂, CF₃ and OCH₃.

6. The compound or a pharmaceutically acceptable salt thereof according to claim 1, wherein Y is selected from —CH₂O—, —CH₂CH₂—, —NH—, —N(CH₃)—, —CH₂S—,

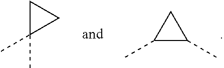

7. The compound or a pharmaceutically acceptable salt thereof according to claim 1, wherein ring A is selected from

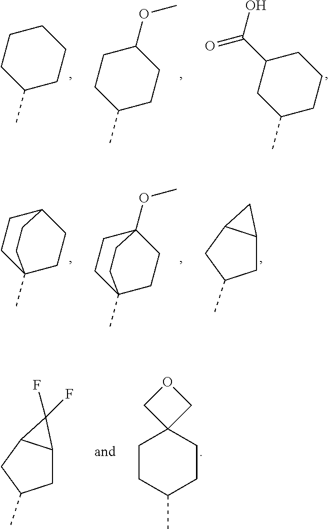

8. The compound or a pharmaceutically acceptable salt thereof according to claim 1, wherein ring B is selected from

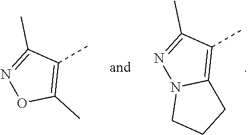

9. The compound or a pharmaceutically acceptable salt thereof according to claim 1, wherein ring C is

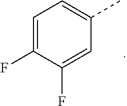

10. The compound or a pharmaceutically acceptable salt thereof according to claim 1, wherein the structural moiety 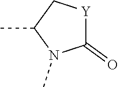
is selected from

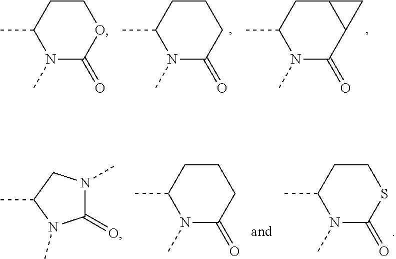

11. The compound or a pharmaceutically acceptable salt thereof according to claim 1, wherein the compound is selected from: 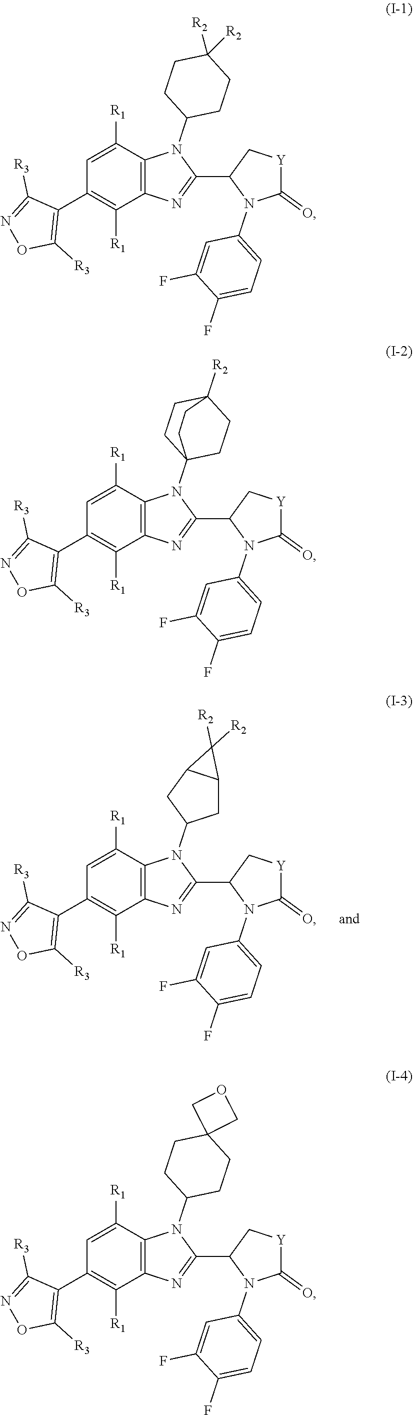
wherein R₁ is as defined in claim 1;
R₂ and R₃ are as defined in claim 1;
Y is as defined in claim 1.

12. A compound represented by the following formula or a pharmaceutically acceptable salt thereof,

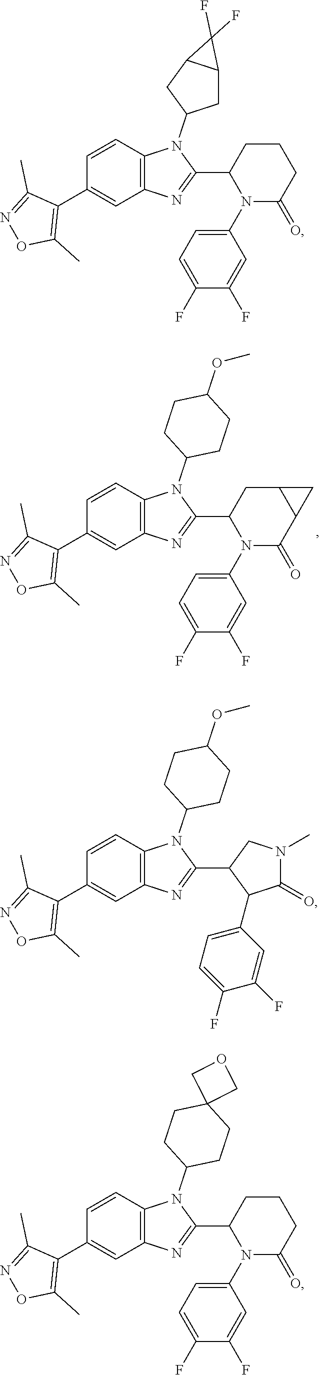

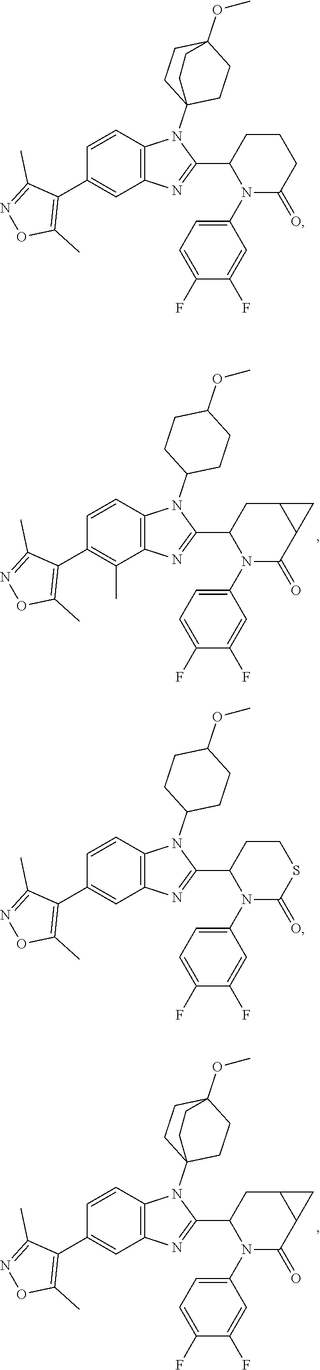

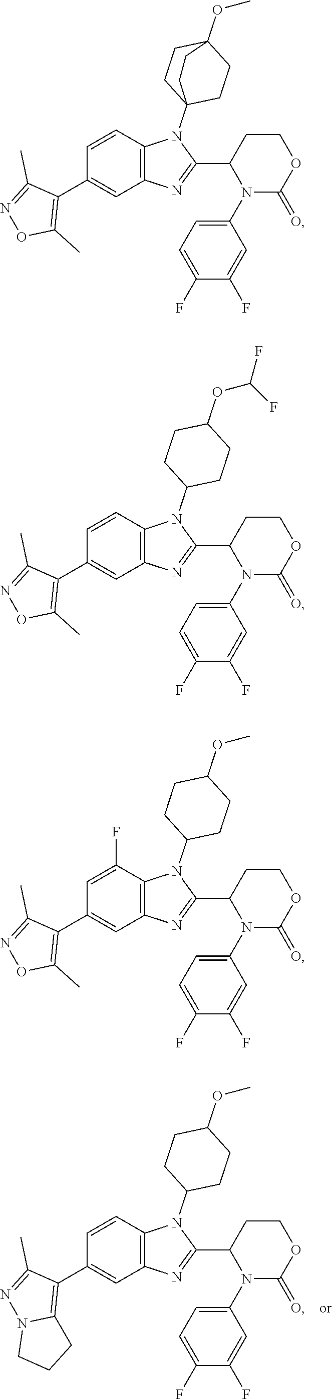

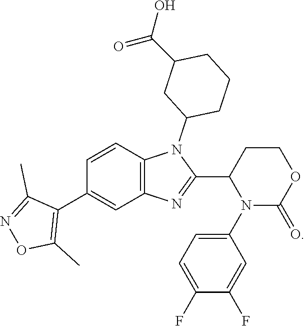 [SMILES: Cc1noc(C)c1-c1ccc2c(c1)nc(C1CCOC(=O)N1c1ccc(F)c(F)c1)n2C1CCCC(C(=O)O)C1]

13. The compound according to claim 12, wherein the compound is selected from 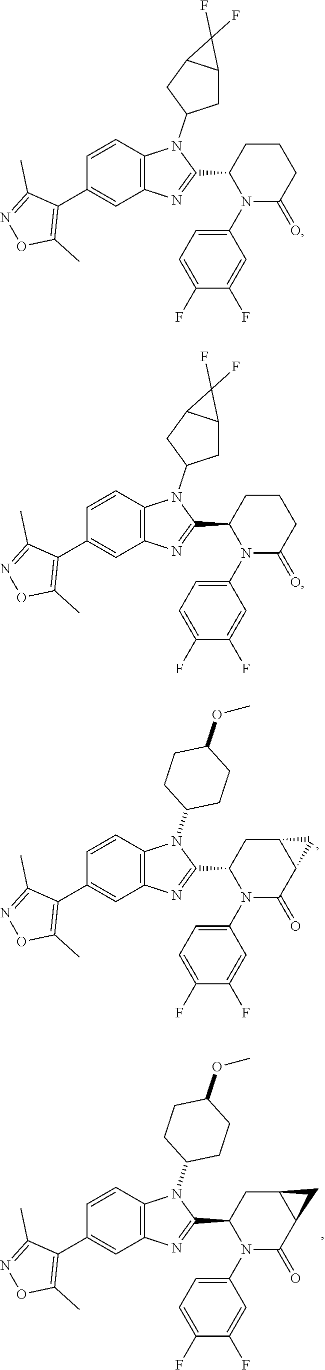 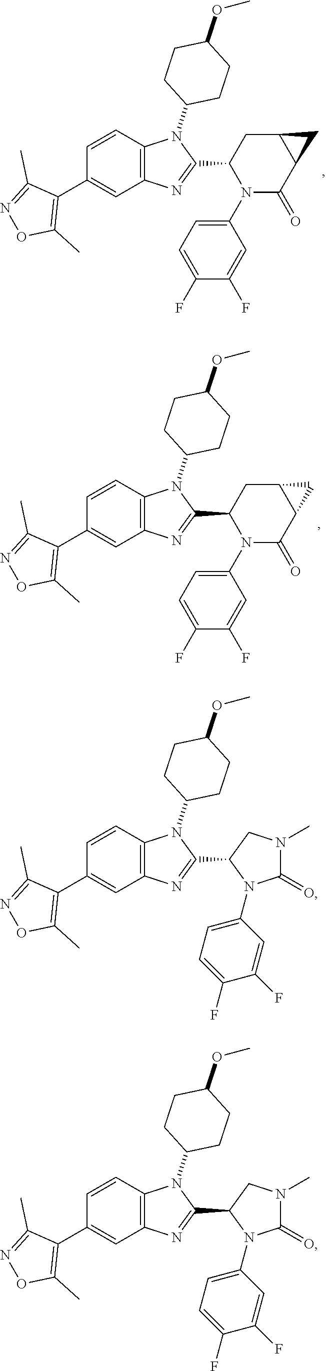 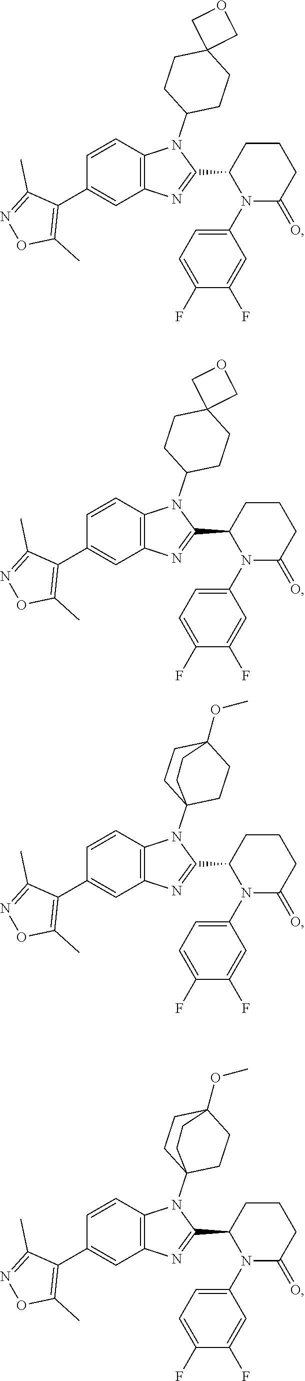 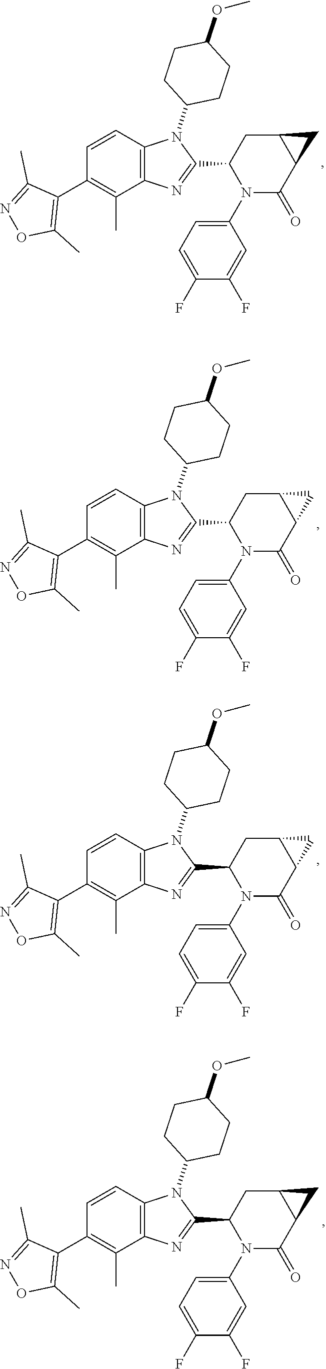 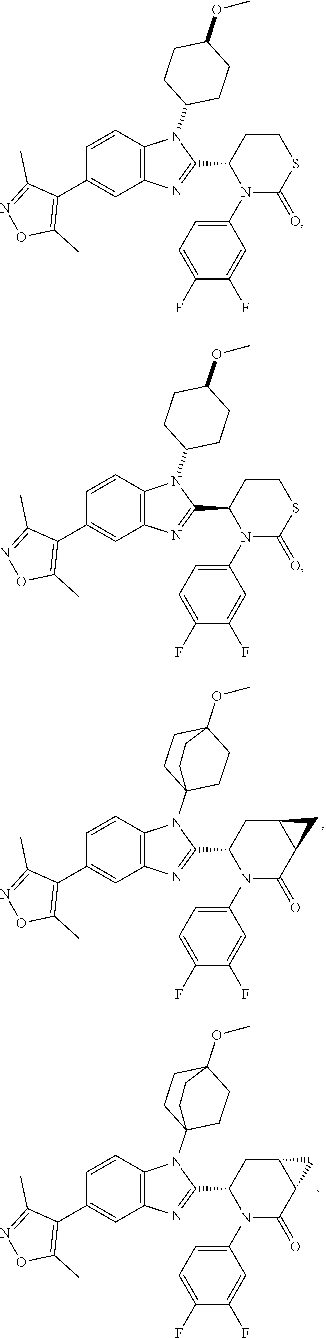 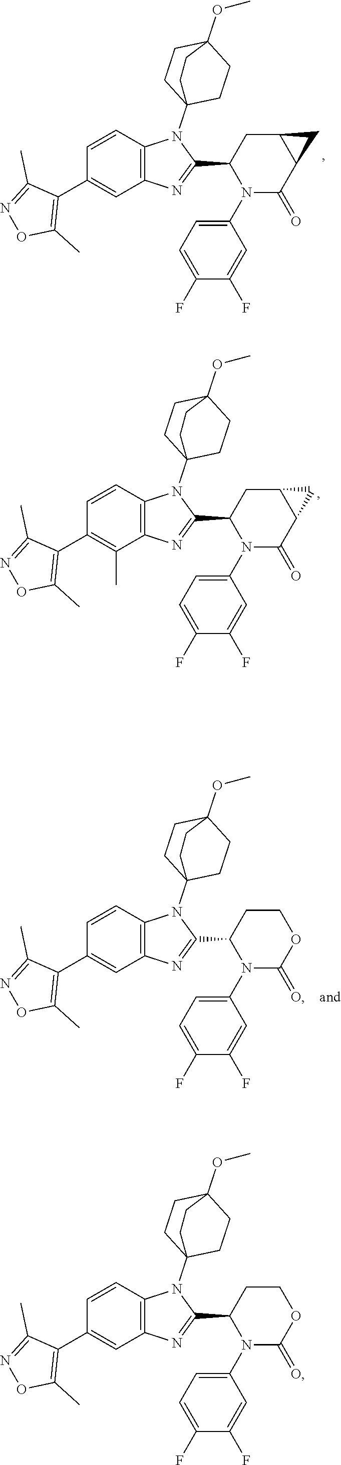 [SMILES: COC12CCC(n3c([C@H]4C[C@H]5C[C@H]5C(=O)N4c4ccc(F)c(F)c4)nc4cc(-c5c(C)noc5C)ccc43)(CC1)CC2 ; COC12CCC(n3c([C@H]4C[C@@H]5C[C@@H]5C(=O)N4c4ccc(F)c(F)c4)nc4c(C)c(-c5c(C)noc5C)ccc43)(CC1)CC2 ; COC12CCC(n3c([C@@H]4CCOC(=O)N4c4ccc(F)c(F)c4)nc4cc(-c5c(C)noc5C)ccc43)(CC1)CC2 ; COC12CCC(n3c([C@H]4CCOC(=O)N4c4ccc(F)c(F)c4)nc4cc(-c5c(C)noc5C)ccc43)(CC1)CC2]
or a pharmaceutically acceptable salt thereof.
